(19) United States
(12) Patent Application Publication
(10) Pub. No.: US 2026/0209229 A1
(43) Pub. Date: Jul. 23, 2026

(54) LSD SALT CRYSTAL FORMS

(71) Applicant: Definium Therapeutics US, Inc., New York, NY (US)

(72) Inventors: Daniel Emil LEVY, San Mateo, CA (US); Stephen E. SCHNEIDER, Raleigh, NC (US)

(21) Appl. No.: 19/420,597

(22) Filed: Dec. 15, 2025

Related U.S. Application Data
(62) Division of application No. 17/729,205, filed on Apr. 26, 2022, now Pat. No. 12,534,460.
(60) Provisional application No. 63/182,700, filed on Apr. 30, 2021.

(51) Int. Cl. C07D 471/04

(52) U.S. Cl. CPC C07D 471/04; C07B 2200/13

(57) ABSTRACT

Polymorphic forms of lysergic acid diethyl amide (LSD) in crystalline salt forms. A pharmaceutical formulation of polymorphic forms of LSD in crystalline salt forms including pharmaceutically acceptable excipients. Polymorphic forms of LSD free-base. Polymorphic forms of a salt form of LSD.

### BACKGROUND OF THE INVENTION

### 1. Technical Field

The present invention relates to compositions lysergic acid diethyl amide (LSD) in salt crystal forms.

### 2. Background Art

Lysergic acid diethyl amide (LSD) is a hallucinogenic drug that was synthesized in 1938 by Albert Hofmann. LSD has the effects in users of altered thoughts, feelings, and awareness of one's surroundings, generally thought to be due to alterations in the serotonin system. LSD can affect 5-HT1A, 5-HT2A, 5-HT2B, 5-HT2c, 5-HT5A, and 5-HT6 receptors, and it may increase glutamate release in the cerebral cortex. LSD can be produced in a crystal form, but then is converted to liquid or formed in tablets, capsules, gelatin squares, or absorbed in paper.

LSD is increasingly used as a therapeutic treatment in a safe setting for anxiety and depression and overcoming addiction. This treatment can be helpful in improving individual's mental health and allowing them to see a bigger picture beyond their own problems. For example, one study has found that LSD can change entrenched patterns of thought and enhanced individuals' feelings of openness, optimism, and mood.

The crystal structure of LSD bound to 5-HT2B was recently discovered. Wacker, et al. (Cell, Jan. 26, 2018) showed that there were conformational rearrangements in the receptor to accommodate LSD and that there is conformational selectivity for LSD's key diethylamide moiety.

In some cases, single pharmaceutical agents can exist in one or more of several unique crystal forms (polymorphs). Specific crystal forms of drugs can be desired in order to provide stability and better handling during processing. Vippangunta, et al. (Adv Drug Deliv Rev. 2001 May 16; 48 (1): 3-26.) state that phase transitions such as polymorph interconversion, desolvation of solvate, formation of hydrate and conversion of crystalline to amorphous form may occur during various pharmaceutical processes, which may alter the dissolution rate and transport characteristics of the drug. Specifically, the various polymorphic forms of pharmaceutical agents may have varying properties related to, for example, particle size, filtration rates, hygroscopicity, ability to micronize, stability, dissolution rates, dispersibility in powders and bioavailability. Polymorph screening can facilitate identification of the polymorphic of a drug with the most favorable solid state and pharmacological profiles. This is particularly important as polymorphic form profiles may differ from free-base to various solid crystalline salt forms. Furthermore, crystalline forms can affect ex vivo and in vivo stability, targeting, solubility, dissolution rate, and bioavailability. Selection of an optimal polymorph can improve performance in the body for compounds that have intrinsic barriers to drug delivery, such as low aqueous solubility, slow dissolution in gastrointestinal media, low permeability, and first-pass metabolism (Yadav, et al. Indian J Pharm Sci, 2009, 71 (4): 359-370).

Because LSD is gaining momentum as a useful therapeutic agent for treatment of mental health indications, various formulations are under development for indication-appropriate administration. Because the various polymorphic forms of LSD salt forms have not been evaluated, formulations currently in development may not be optimal for their intended usages. Therefore, there remains a need for the identification and characterization of LSD polymorphic forms and for the identification of the polymorphic forms of crystalline salt forms of LSD.

### SUMMARY OF THE INVENTION

The present invention provides for polymorphic forms of lysergic acid diethyl amide (LSD) in crystalline salt forms.

The present invention also provides for a pharmaceutical formulation of polymorphic forms of LSD in crystalline salt forms including pharmaceutically acceptable excipients.

In one embodiment, the present invention provides for polymorphic forms of LSD free-base.

In one embodiment, the present invention provides for polymorphic forms of a salt form of LSD.

In one embodiment, the salt form of LSD comprises LSD and an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, L-ascorbic acid, D-ascorbic acid, L-aspartic acid, D-aspartic acid, benzenesulfonic acid, benzoic acid, (+)-camphoric acid, (−) camphoric acid, (+)-camphor-10-sulfonic acid, (−)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, D-glucoheptonic acid, L-glucoheptonic acid, D-gluconic acid, L-gluconic acid, D-glucuronic acid, L-glucuronic acid, L-glutamic acid, D-glutamic acid, glutaric acid, glycerophosphoric acid, glycolic acid, hippuric acid, hydrobromic acid, hydrochloric acid, isobutyric acid, D,L-lactic acid, D-lactic acid, L-lactic acid, lactobionic acid, lauric acid, maleic acid, L-malic acid, D-malic acid, malonic acid, D,L-mandelic acid, D-mandelic acid, L-mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, L-pyroglutamic acid, D-pyroglutamic acid, salicylic acid, sebacic acid, stearic acid, succinic acid, sulfuric acid, L-tartaric acid, D-tartaric acid, meso-tartaric acid, thiocyanic acid, para-toluenesulfonic acid, undecylenic acid and xinafoic acid.

### DESCRIPTION OF THE DRAWINGS

Other advantages of the present invention are readily appreciated as the same becomes better understood by reference to the following detailed description when considered in connection with the accompanying drawings wherein:

FIG. 1 is an XPRD diffractogram of D-LSD Naphthalene-2-sulfonic acid salt;

FIG. 2 is an XRPD diffractogram of D-LSD tartaric acid (+L) hemi-salt;

FIG. 3 is an XRPD diffractogram of D-LSD maleic acid salt form A;

FIG. 4 is an XRPD diffractogram of D-LSD maleic acid salt form B;

FIG. 5 is an XRPD diffractogram of D-LSD xinafoic acid salt form A;

FIG. 6 is an XRPD diffractogram of D-LSD xinafoic acid salt form A′;

FIG. 7 is an XRPD diffractogram of D-LSD phosphoric acid salt form A;

FIG. 8 is an XRPD diffractogram of D-LSD naphthalene-1,5-disulfonic acid hemi-salt;

FIG. 9 is an XRPD diffractogram of D-LSD oxalic acid salt form A;

FIG. 10 is an XRPD diffractogram of D-LSD benzoic acid salt form A;

FIG. 11 is an XRPD diffractogram of D-LSD benzoic acid salt form B;

FIG. 12 is an XRPD diffractogram of D-LSD fumaric acid salt;

FIG. 13 is an XRPD diffractogram of D-LSD fumaric acid salt hemi-salt;

FIG. 14 is an XRPD diffractogram of D-LSD ascorbic acid (L) salt form A;

FIG. 15 is an XRPD diffractogram of D-LSD ascorbic acid (L) salt form B;

FIG. 16 is an XRPD diffractogram of D-LSD D-tartaric acid salt;

FIG. 17 is an XRPD diffractogram of D-LSD hydrochloric acid salt form A;

FIG. 18 is an XRPD diffractogram of D-LSD hydrochloric acid salt form A′;

FIG. 19 is an XRPD diffractogram of D-LSD hydrochloric acid salt form B;

FIG. 20 is an XRPD diffractogram of D-LSD hydrochloric acid salt form C;

FIG. 21 is an XRPD diffractogram of D-LSD hydrochloric acid salt form D;

FIG. 22 is an XRPD diffractogram of D-LSD hydrochloric acid salt form E;

FIG. 23 is the XRPD diffractograms of D-LSD free-base form A (bottom) and form B (top);

FIG. 24 is the molecular structure of D-LSD (free base);

FIG. 25 shows DSC and TGA thermographs of D-LSD benzoate salt pattern A;

FIG. 26 shows DSC and TGA thermographs of D-LSD benzoate salt pattern A′;

FIG. 27 is an NMR of D-LSD free base form B;

FIG. 28 is DSC and TGA thermographs of D-LSD tartrate salt;

FIG. 29 is DSC and TGA thermographs of D-LSD phosphate salt form B;

FIG. 30 is an ¹H NMR of D-LSD phosphoric acid salt form B;

FIG. 31 is an ¹H NMR of D-LSD D-tartaric acid salt;

FIG. 32 shows DSC and TGA thermographs of D-LSD phosphoric acid salt form A;

FIG. 33 shows DSC and TGA thermographs of D-LSD maleic acid salt form A;

FIG. 34 shows DSC and TGA thermographs of D-LSD maleic acid salt form A (peak at 145° C.) and form B (peak at 199° C.);

FIG. 35 is a DVS profile of D-LSD D-tartaric acid salt;

FIG. 36 is a DSC thermogram of D-LSD phosphoric acid salt Pattern C;

FIG. 37 is an overlay of XRPD Pattern of D-LSD phosphate salt Pattern C;

FIG. 38 shows DSC and TGA thermographs of amorphous D-LSD oxalate salt;

FIG. 39 shows XRPD patterns of amorphous D-LSD oxalate salt;

FIG. 40 is a DSC thermogram of D-LSD oxalate Pattern B;

FIG. 41 is a DSC thermogram of D-LSD oxalate Pattern C;

FIG. 42 is a TGA thermogram of D-LSD oxalate Pattern C;

FIG. 43 is a DSC thermogram of D-LSD oxalate Pattern E;

FIG. 44 is a TGA thermogram of D-LSD oxalate Pattern E;

FIG. 45 is a DSC thermogram of D-LSD oxalate Pattern G;

FIG. 46 is a TGA thermogram of D-LSD oxalate Pattern G;

FIG. 47 shows XRPD patterns of D-LSD oxalate salt Pattern A (bottom), Pattern B, Pattern C, Pattern D, Pattern E, Pattern F and Pattern G (top);

FIG. 48 is an XRPD Pattern of amorphous D-LSD phosphate salt;

FIG. 49 is a DSC thermogram of amorphous D-LSD phosphate salt;

FIG. 50 is a DSC thermogram of D-LSD Phosphate Salt Pattern D;

FIG. 51 is a TGA thermogram of D-LSD Phosphate Salt Pattern D;

FIG. 52 is a ¹H NMR spectrum of D-LSD Phosphate Salt Pattern D);

FIG. 53 is a DSC thermogram of D-LSD Phosphate Salt Pattern B;

FIG. 54 shows XRPD patterns of D-LSD HBr Pattern A (bottom); D-LSD HBr Pattern B (middle); D-LSD HBr Pattern C (top);

FIG. 55 is a DSC/TGA thermogram of D-LSD HBr salt Pattern C;

FIG. 56 is a ¹H NMR spectrum for D-LSD HBr salt Pattern C;

FIG. 57 is an XRPD pattern of D-LSD Hydrobromide salt Pattern A starting material (bottom) and after stress at 40° C./75 RH (top);

FIG. 58 is an XRPD overlay D-LSD Phosphate salt Pattern A (bottom), Pattern B (second), Pattern C (third), Pattern D (fourth), and Pattern E (top)

FIG. 59 shows DSC/TGA thermograms for D-LSD phosphate salt Pattern E;

FIG. 60 is a ¹H NMR spectrum of D-LSD phosphate Pattern E;

FIG. 61 is an XRPD pattern for D-LSD sulfate amorphous salt;

FIG. 62 is a DSC thermogram for D-LSD sulfate amorphous salt;

FIG. 63 is a ¹H NMR spectrum of D-LSD sulfate salt;

FIG. 64 is a ¹H NMR overlap between D-LSD Free Base (bottom) and D-LSD sulfate salt (top);

FIG. 65 is an XRPD pattern of D-LSD hydrobromide amorphous salt;

FIG. 66 is a ¹H NMR spectrum of D-LSD hydrobromide salt;

FIG. 67 is a ¹H NMR overlap between D-LSD Free Base (bottom) and D-LSD Hydrobromic salt (top);

FIG. 68 is a DVS profile of D-LSD oxalate Pattern B;

FIG. 69 is XRPD patterns of D-LSD oxalate Pattern B ex. DVS post equilibration at 0% RH (middle trace) and 90% RH (top trace) compared to Pattern B reference (bottom trace);

FIG. 70 shows XRPD patterns of D-LSD oxalate Pattern B after storage at 40° C./75% RH, (top), versus initial (bottom);

FIG. 71 shows XRPD patterns of D-LSD phosphate Pattern C after storage at 40° C./75% RH (top) versus initial (bottom);

FIG. 72 shows XRPD patterns of amorphous D-LSD sulfate salt

FIG. 73 is a DSC thermogram of D-LSD HBr salt;

FIG. 74 shows XRPD patterns of D-LSD HBr Pattern A versus D-LSD Free Base (bottom);

FIG. 75 shows an XRPD pattern of D-LSD Free Base Pattern B (bottom), D-LSD HBr Pattern A (middle), and D-LSD HBr Pattern B isolated from toluene (top);

FIG. 76 shows DSC/TGA thermograms of D-LSD HBr Pattern B;

FIG. 77 shows a ¹H NMR of D-LSD HBr Pattern B;

FIG. 78 shows DSC/TGA thermograms of D-LSD sulfate salt;

FIG. 79 shows DSC/TGA thermograms of D-LSD HBr amorphous salt;

FIG. 80 is a DSC/TGA thermograms of D-LSD sulfate Pattern A;

FIG. 81 is a ¹H NMR spectrum of D-LSD sulfate Pattern A;

FIG. 82 shows DSC/TGA thermograms of D-LSD HBr Pattern A salt;

and

FIG. 83 shows XRPD patterns of D-LSD sulfate Pattern A obtained after the mixed solvent cooling crystallisation experiments.

### DETAILED DESCRIPTION OF THE INVENTION

The present invention provides for polymorphic forms of lysergic acid diethyl amide (LSD) in crystalline salt forms.

The crystalline salt forms include free-base LSD, which is a basic amine, optionally along with an acid.

The crystalline forms can be an anhydrate, a hydrate, or a solvate. A hydrate can have a molecular ratio of water to LSD of about 0.25 to about 10. The solvate can include an organic solvent such as methanol, ethanol, isopropanol, tetrahydrofuran, dioxane, ethyl acetate, hexane, benzene, toluene, xylene, tert-butyl methyl ether, diethyl ether, dibutyl ether, acetone, anisole, isopropyl acetate, methylethyl ketone, 1-propanol, or tert-butylmethyl ether. The form can also be amorphous.

The acid can be, but is not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, L-ascorbic acid, D-ascorbic acid, L-aspartic acid, D-aspartic acid, benzenesulfonic acid, benzoic acid, (+)-camphoric acid, (−)-camphoric acid, (+)-camphor-10-sulfonic acid, (−)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, D-glucoheptonic acid, L-glucoheptonic acid, D-gluconic acid, L-gluconic acid, D-glucuronic acid, L-glucuronic acid, L-glutamic acid, D-glutamic acid, glutaric acid, glycerophosphoric acid, glycolic acid, hippuric acid, hydrobromic acid, hydrochloric acid, isobutyric acid, D,L-lactic acid, D-lactic acid, L-lactic acid, lactobionic acid, lauric acid, maleic acid, L-malic acid, D-malic acid, malonic acid, D,L-mandelic acid, D-mandelic acid, L-mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, L-pyroglutamic acid, D-pyroglutamic acid, salicylic acid, sebacic acid, stearic acid, succinic acid, sulfuric acid, L-tartaric acid, D-tartaric acid, meso-tartaric acid, thiocyanic acid, para-toluenesulfonic acid, undecylenic acid and xinafoic acid. Any stereoisomers of the acids can also be used, i.e. (L) or (D) or (D,L), (+) or (−), or (meso) where appropriate. Salt stoichiometry can be used for acids such as tartaric acid or citric acid or other di- or tri-carboxylic acids, di- or tri-sulfonic acids or others generally known to one skilled in the art (LSD: acid of 1:0.5, 1:1, 2:1, 3:1, etc.).

It is understood by one skilled in the art that other acids are useful for the preparation of salt forms of LSD. It is further understood by one skilled in the art that said salt forms of LSD may exist in multiple polymorphic forms.

It is understood by one skilled in the art that salt forms of LSD may exist as anhydrates, hydrates, solvates and co-crystals. It is further understood by one skilled in the art that said hydrates, solvates and co-crystals may exist in multiple polymorphic forms. The salt can have a molecular ratio of an acid to LSD of from about 0.25 to about 3.

It is understood by one skilled in the art that salt forms of LSD may exist with varying stoichiometric ratios between the LSD free-base and the acid component of an LSD salt form. One skilled in the art will recognize that this property is not necessarily dependent upon the number of acidic residues present on the acid unit. For example, a mono-acid may form a mono-salt a hemi-salt or an alternate free-base/acid combination. Likewise, a di-acid may form a hemi-salt, a mono-salt, or an alternate free-base/acid combination. It is further understood by one skilled in the art that salt forms with differing stoichiometric ratios between the free-base and the acid components are unique salt forms and may exist in different polymorphic forms.

The LSD crystalline salt forms can be characterized and identified by X-ray powder diffraction (XPRD) signals, melting points, differential scanning calorimetry (DSC), thermogravimetric analysis (TGA), infrared spectroscopy (IR), or other methods known to one skilled in the art.

The LSD crystalline salt forms can be made by various methods such as, but not limited to, slurry at room temperature, solid vapor diffusion (diffusion of a vapor of a volatile solvent, in which the LSD is not soluble, into a solvent containing the sample to be crystallized), temperature cycling (successive heating-cooling cycles for dissolution and recrystallization), slow evaporation at different temperatures, liquid vapor diffusion, polymer induced crystallization, anti-solvent addition (adding a liquid miscible with a solvent that reduces LSD solubility in the new mixed solvent), grinding, or other methods known to one skilled in the art.

In one embodiment, the present invention provides for polymorphic forms of lysergic acid diethyl amide (LSD) in crystalline salt forms. In some embodiments, said polymorphic forms of LSD may be LSD free-base, a hydrate of LSD free-base or a solvate of LSD free-base. In some embodiments, the polymorphic form of LSD is a hydrate of LSD. In some embodiments, the polymorphic form of LSD is a solvate of LSD. In some embodiments, the polymorphic form of LSD is the free-base of LSD.

In one embodiment, the present invention provides for polymorphic forms of a salt form of LSD. In some embodiments, said polymorphic forms of a salt form of LSD may be a salt form of LSD, a hydrate of a salt form of LSD, a solvate of a salt form of LSD or a co-crystal of LSD comprising LSD free-base and an acid. In some embodiments, the polymorphic form of a salt form of LSD is a hydrate of a salt form of LSD. In some embodiments, the polymorphic form of a salt form of LSD is a solvate of a salt form of LSD. In some embodiments, the polymorphic form of LSD is a co-crystal of LSD comprising LSD free-base and an acid.

The EXAMPLES below show several different polymorphs. D-LSD phosphate salt can be pattern A, pattern B, pattern C, pattern D, pattern E, pattern F, or pattern G. D-LSD oxalate salt can be pattern A, pattern B, pattern C, pattern D, pattern E, pattern F, pattern G, or pattern H. D-LSD sulfate salt can be pattern A. D-LSD HBr salt can be pattern A, pattern B, or pattern C.

In one embodiment, the salt form of LSD comprises LSD and an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, L-ascorbic acid, D-ascorbic acid, L-aspartic acid, D-aspartic acid, benzenesulfonic acid, benzoic acid, (+)-camphoric acid, (−)-camphoric acid, (+)-camphor-10-sulfonic acid, (−)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, D-glucoheptonic acid, L-glucoheptonic acid, D-gluconic acid, L-gluconic acid, D-glucuronic acid, L-glucuronic acid, L-glutamic acid, D-glutamic acid, glutaric acid, glycerophosphoric acid, glycolic acid, hippuric acid, hydrobromic acid, hydrochloric acid, isobutyric acid, D,L-lactic acid, D-lactic acid, L-lactic acid, lactobionic acid, lauric acid, maleic acid, L-malic acid, D-malic acid, malonic acid, D,L-mandelic acid, D-mandelic acid, L-mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, L-pyroglutamic acid, D-pyroglutamic acid, salicylic acid, sebacic acid, stearic acid, succinic acid, sulfuric acid, L-tartaric acid, D-tartaric acid, meso-tartaric acid, thiocyanic acid, para-toluenesulfonic acid, undecylenic acid and xinafoic acid.

In a preferred embodiment, the salt form of LSD comprises LSD and an acid selected from acetic acid, hydrochloric acid, methanesulfonic acid, maleic acid, L-malic acid, D-malic acid, meso-tartaric acid, L-tartaric acid, D-tartaric acid, citric acid, succinic acid, para-toluenesulfonic acid, sulfuric acid, phosphoric acid, benzenesulfonic acid, xinafoic acid, hydrobromic acid, oxalic acid, L-aspartic acid, D-aspartic acid, naphthalene-1,5-disulfonic acid, L-glutamic acid, D-glutamic acid, malonic acid, fumaric acid, D-glucuronic acid, D-gluconic acid, glycolic acid, L-ascorbic acid, naphthalene-2-sulfonic acid, benzoic acid and ethane-1,2-disulfonic acid.

In a more preferred embodiment, the salt form of LSD comprises LSD and an acid selected from hydrochloric acid, methanesulfonic acid, maleic acid, meso-tartaric acid, L-tartaric acid, D-tartaric acid, phosphoric acid, benzenesulfonic acid, xinafoic acid, hydrobromic acid, oxalic acid, naphthalene-1,5-disulfonic acid, fumaric acid, L-ascorbic acid, naphthalene-2-sulfonic acid, benzoic acid, L-malic acid, citric acid, malonic acid and sulfuric acid.

In a most preferred embodiment, the salt form of LSD comprises LSD and an acid selected from maleic acid, L-tartaric acid, D-tartaric acid, phosphoric acid, xinafoic acid, oxalic acid, naphthalene-1,5-disulfonic acid, naphthalene-2-sulfonic acid, L-ascorbic acid, fumaric acid and benzoic acid. In some embodiments, the salt form of LSD comprises LSD and L-tartaric acid. In some embodiments, the salt form of LSD comprises LSD and D-tartaric acid. In some embodiments, the salt form of LSD comprises LSD and phosphoric acid. In some embodiments, the salt form of LSD comprises LSD and xinafoic acid. In some embodiments, the salt form of LSD comprises LSD and oxalic acid. In some embodiments, the salt form of LSD comprises LSD and naphthalene-1,5-disulfonic acid. In some embodiments, the salt form of LSD comprises LSD and fumaric acid. In some embodiments, the salt form of LSD comprises LSD and naphthalene-2-sulfonic acid. In some embodiments, the salt form of LSD comprises LSD and L-ascorbic acid. In some embodiments, the salt form of LSD comprises LSD and benzoic acid.

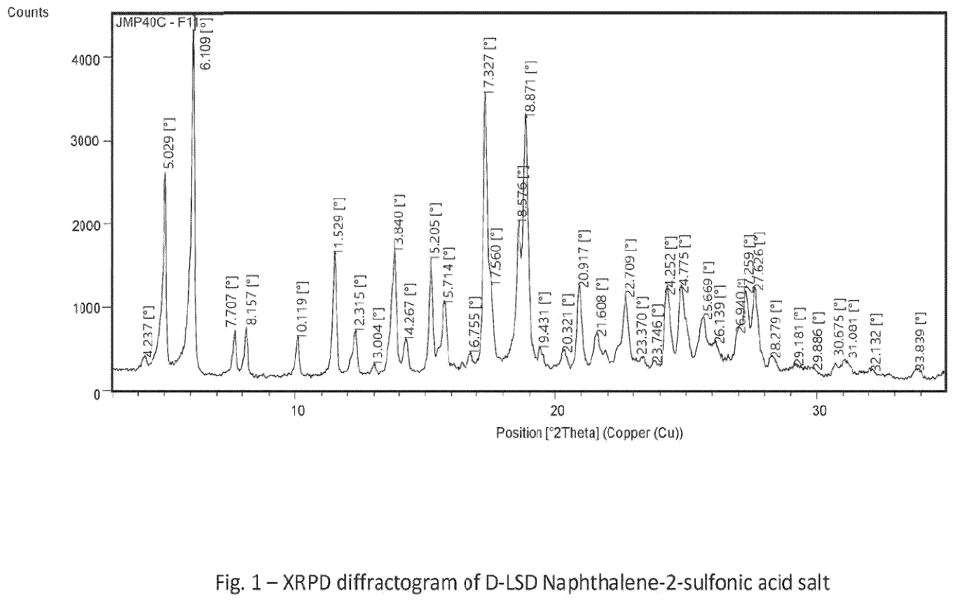

The naphthalene-2-sulfonic acid salt of D-LSD is characterized by an x-ray powder diffraction pattern obtained by irradiation with Cu Kα x-rays having peaks expressed as 2θ at about 5.0, about 6.1, about 11.5, about 17.3 and about 18.9 (FIG. 1).

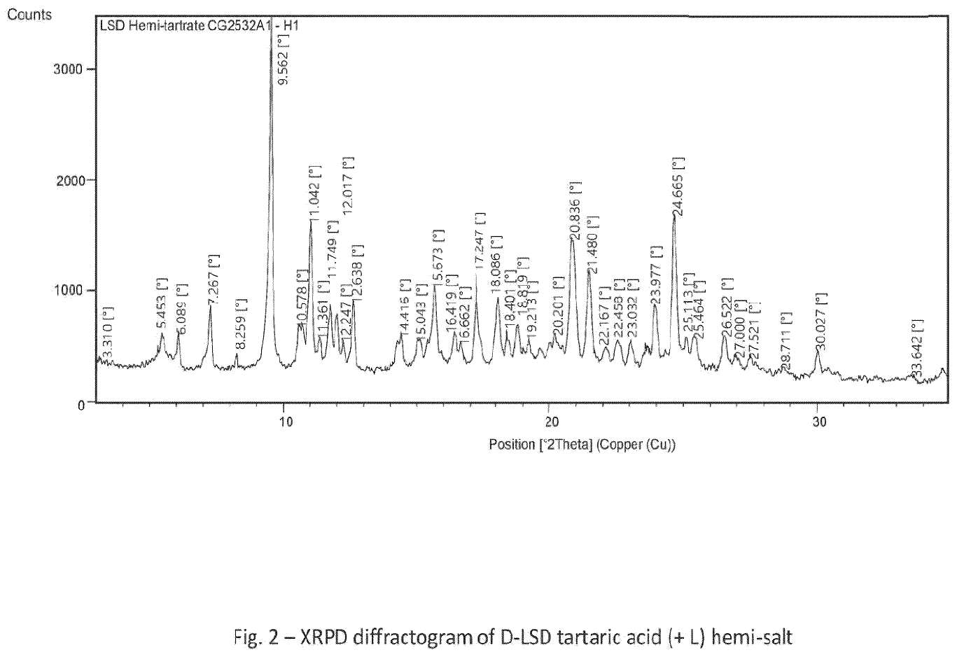

The L-tartaric acid hemi-salt of D-LSD is characterized by an x-ray powder diffraction pattern obtained by irradiation with Cu Kα x-rays having peaks expressed as 2θ at about 9.6, about 11, about 20.8 and about 24.7 (FIG. 2).

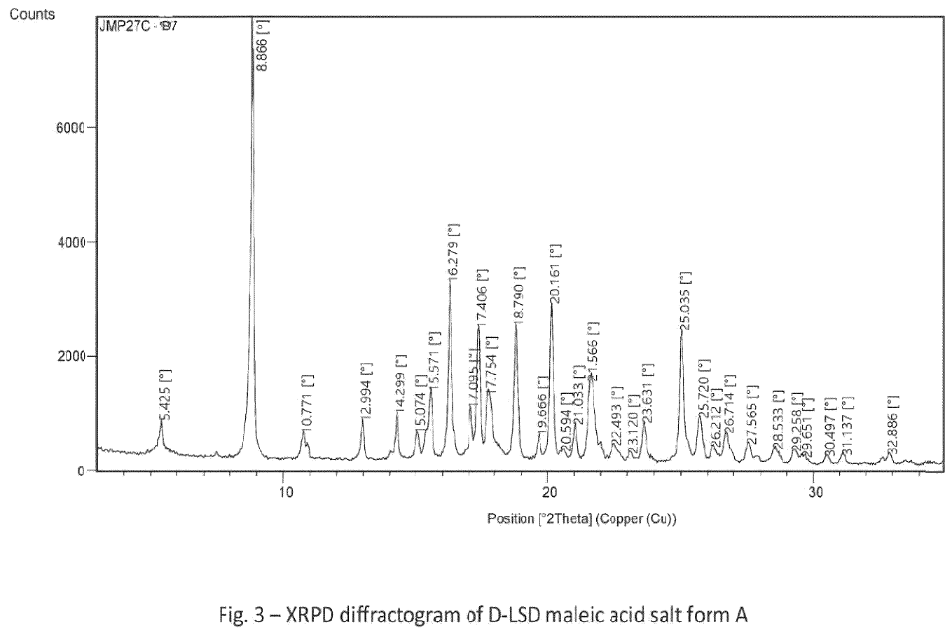

The maleic acid salt form A of D-LSD is characterized by an x-ray powder diffraction pattern obtained by irradiation with Cu Kα x-rays having peaks expressed as 2θ at about 8.8, about 16.2, about 17.4, about 18.8, about 20.1 and about 25.0 (FIG. 3).

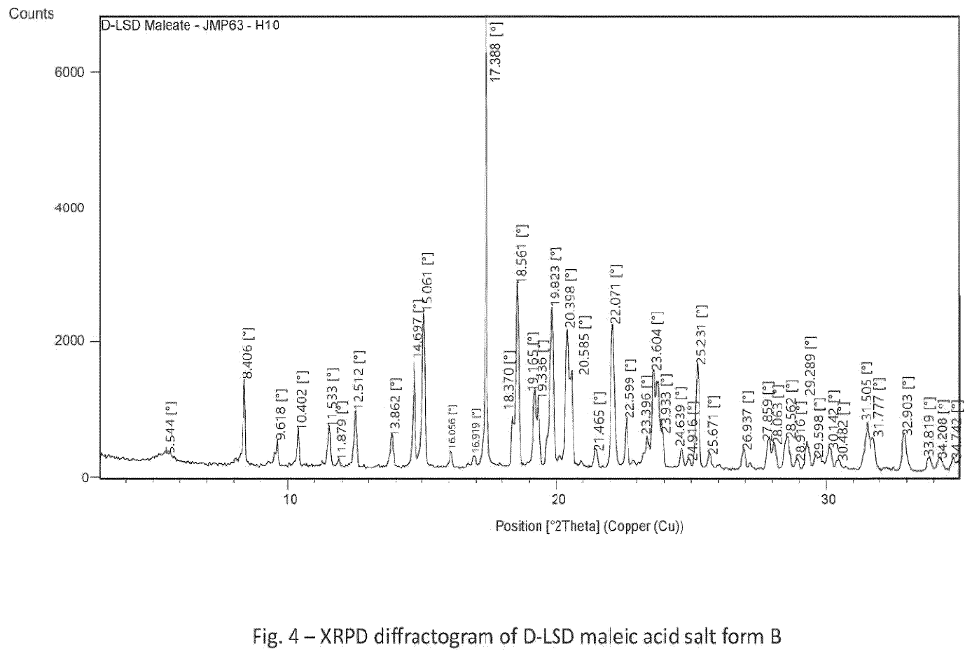

The maleic acid salt form B of D-LSD is characterized by an x-ray powder diffraction pattern obtained by irradiation with Cu Kα x-rays having peaks expressed as 2θ at about 8.4, about 14.7, about 15.1, about 17.4 and about 22.1 (FIG. 4).

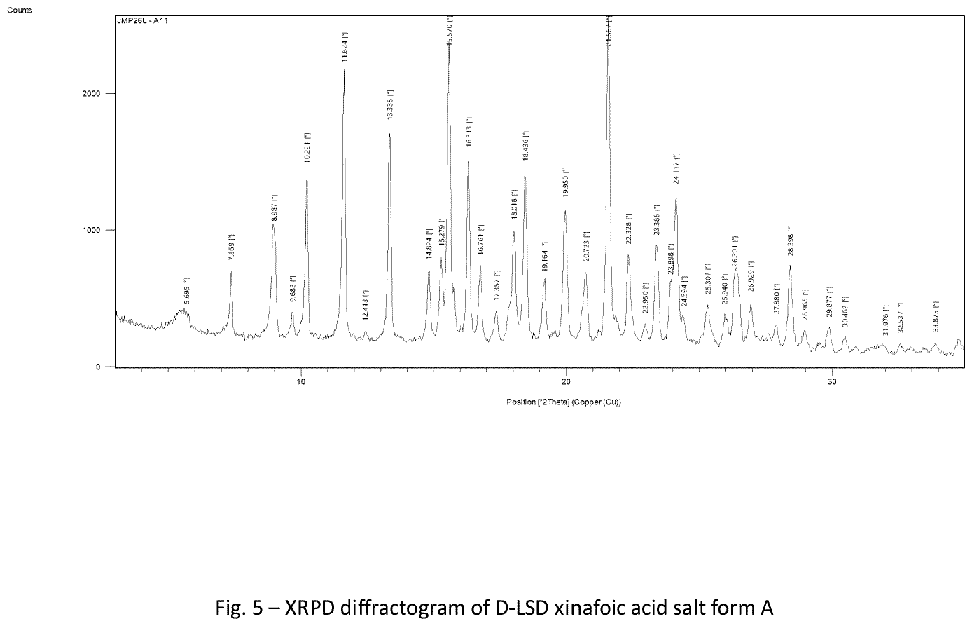

The xinafoic acid salt form A of D-LSD is characterized by an x-ray powder diffraction pattern obtained by irradiation with Cu Kα x-rays having peaks expressed as 2θ at about 10.2, about 11.6, about 13.3, about 15.6 and about 21.6 (FIG. 5).

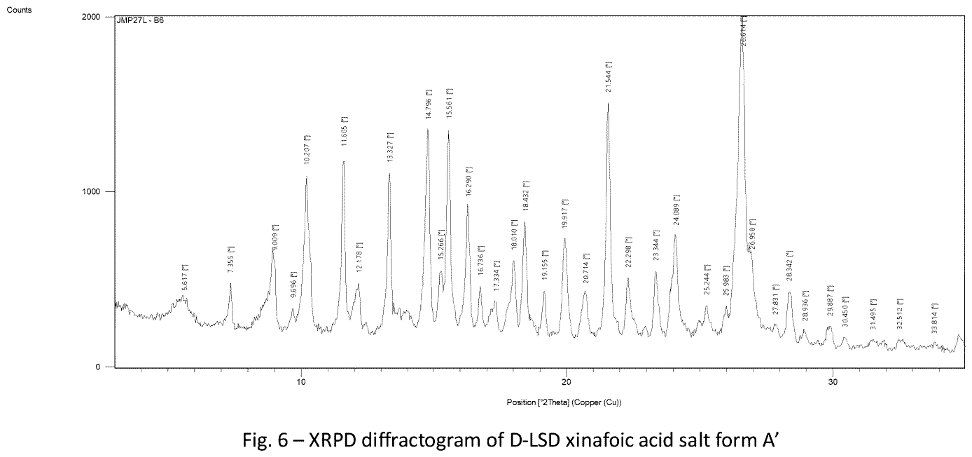

The xinafoic acid salt form A′ of D-LSD is characterized by an x-ray powder diffraction pattern obtained by irradiation with Cu Kα x-rays having peaks expressed as 2θ at about 14.7, about 15.6, about 21.5 and about 26.6 (FIG. 6).

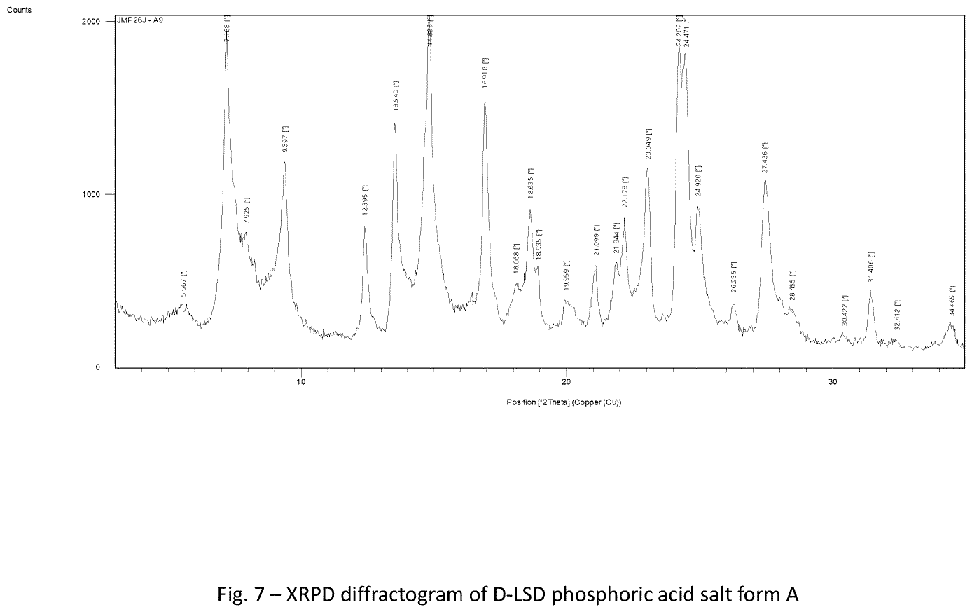

The phosphoric acid salt form A of D-LSD is characterized by an x-ray powder diffraction pattern obtained by irradiation with Cu Kα x-rays having peaks expressed as 2θ at about 7.2, about 13.5, about 16.9, about 23.0 and about 24.5 (FIG. 7).

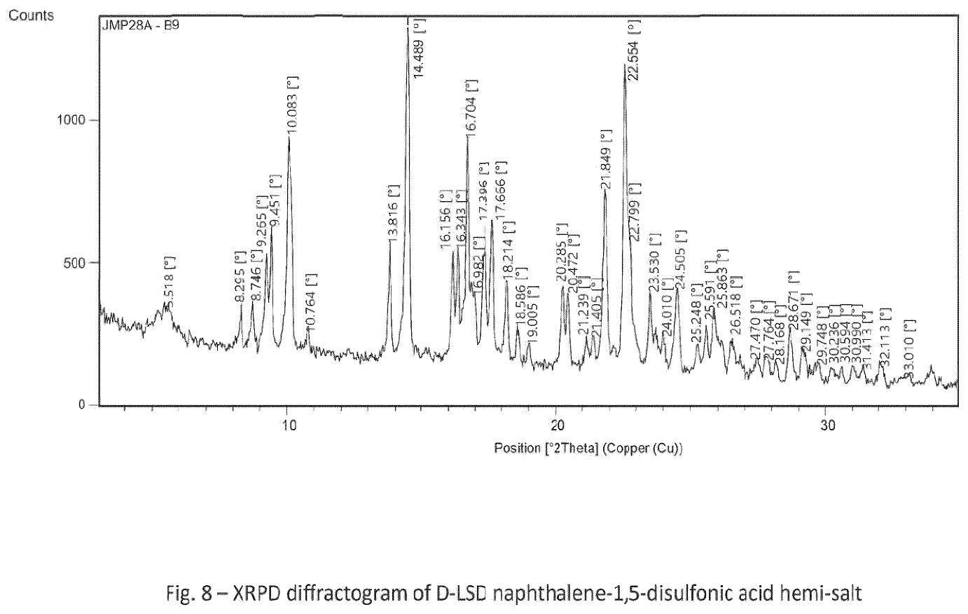

The naphthalene-1,5-disulfonic acid hemi-salt of D-LSD is characterized by an x-ray powder diffraction pattern obtained by irradiation with Cu Kα x-rays having peaks expressed as 2θ at about 10.1, about 14.4, about 16.7, about 23.5, about 24.5, about 21.9 and about 22.6 (FIG. 8).

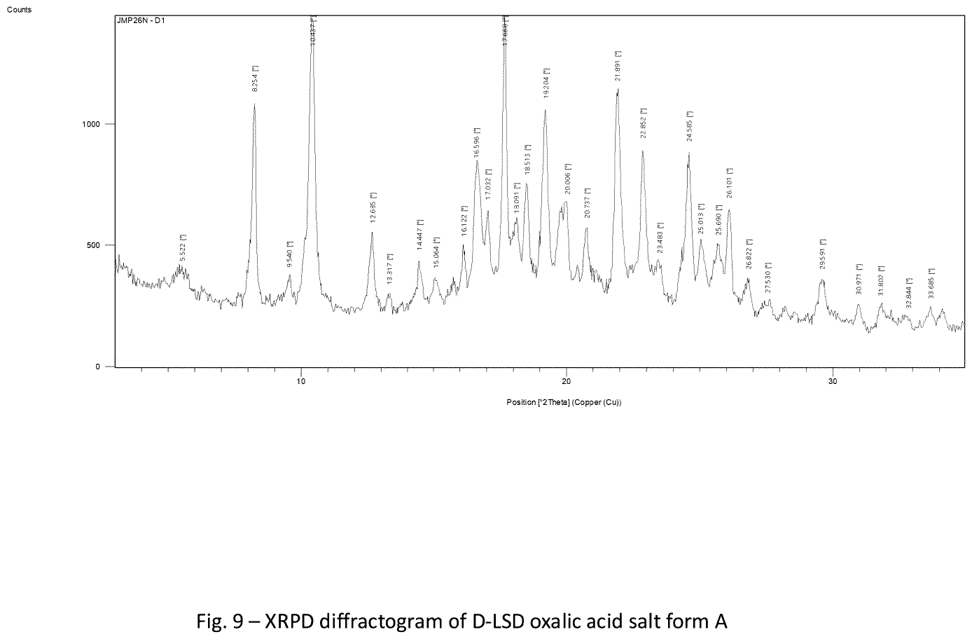

The oxalic acid salt form A of D-LSD is characterized by an x-ray powder diffraction pattern obtained by irradiation with Cu Kα x-rays having peaks expressed as 2θ at about 8.2, about 10.4, about 17.7, about 19.2 and about 21.9 (FIG. 9).

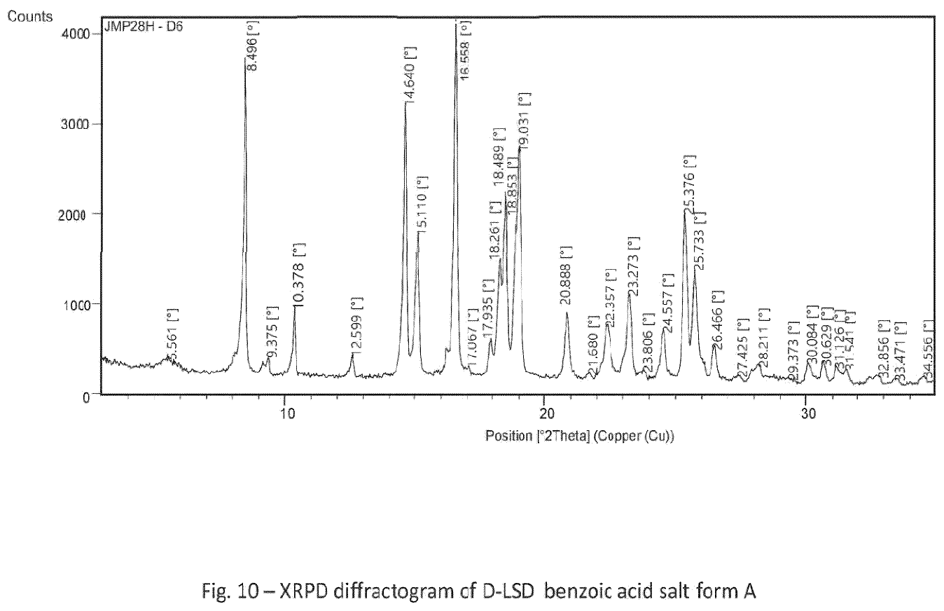

The benzoic acid salt form A of D-LSD is characterized by an x-ray powder diffraction pattern obtained by irradiation with Cu Kα x-rays having peaks expressed as 2θ at about 8.5, about 14.6, about 16.6, about 18.5 and about 19.0 (FIG. 10).

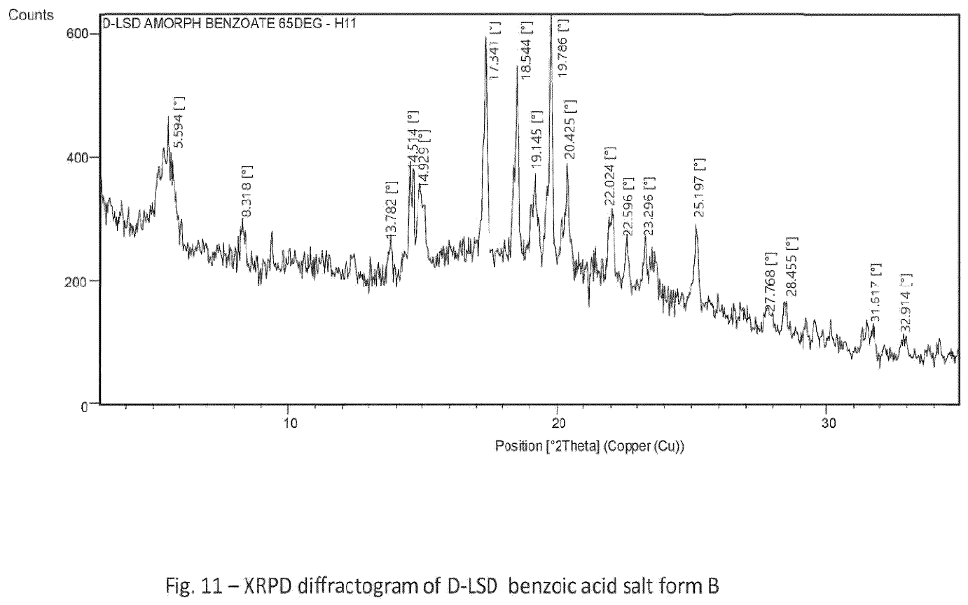

The benzoic acid salt form B of D-LSD is characterized by an x-ray powder diffraction pattern obtained by irradiation with Cu Kα x-rays having peaks expressed as 2θ at about 17.3, about 19.8, about 20.4 and about 25.2 (FIG. 11).

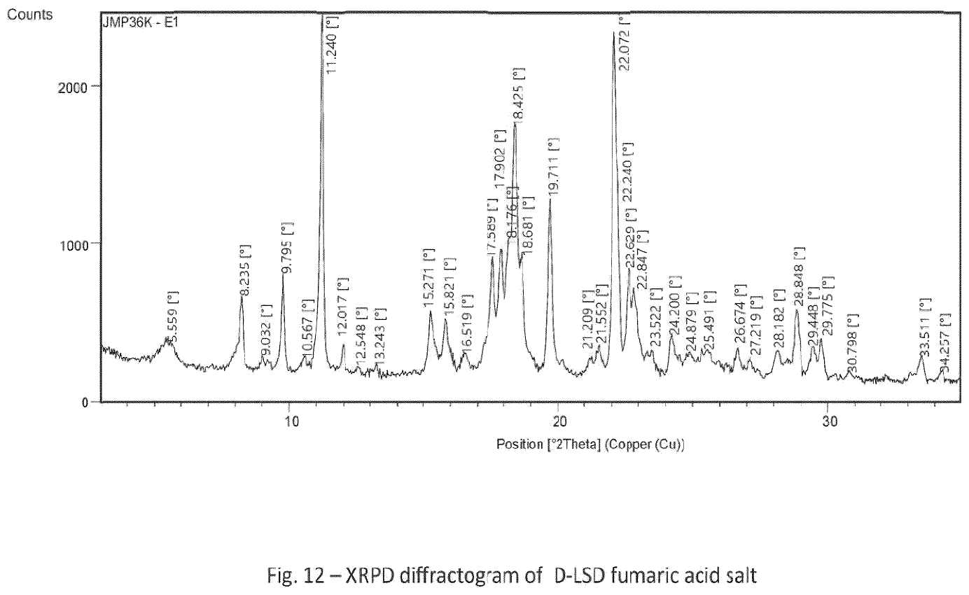

The fumaric acid salt of D-LSD is characterized by an x-ray powder diffraction pattern obtained by irradiation with Cu Kα x-rays having peaks expressed as 2θ at about 11.2, about 18.4, about 19.7 and about 22.1 (FIG. 12).

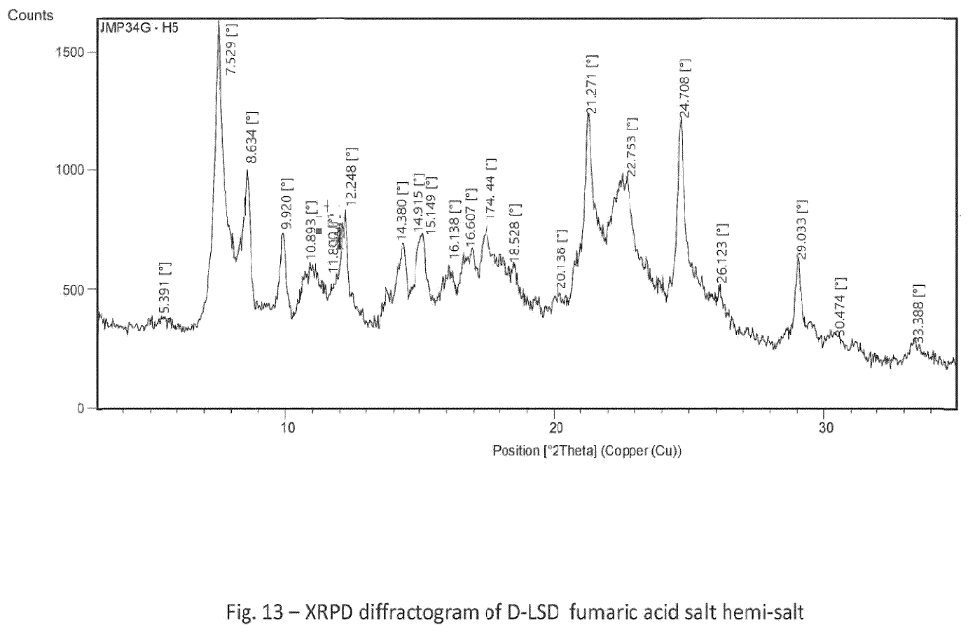

The fumaric acid hemi-salt of D-LSD is characterized by an x-ray powder diffraction pattern obtained by irradiation with Cu Kα x-rays having peaks expressed as 2θ at about 7.5, about 21.3, about 22.7 and about 24.7 (FIG. 13).

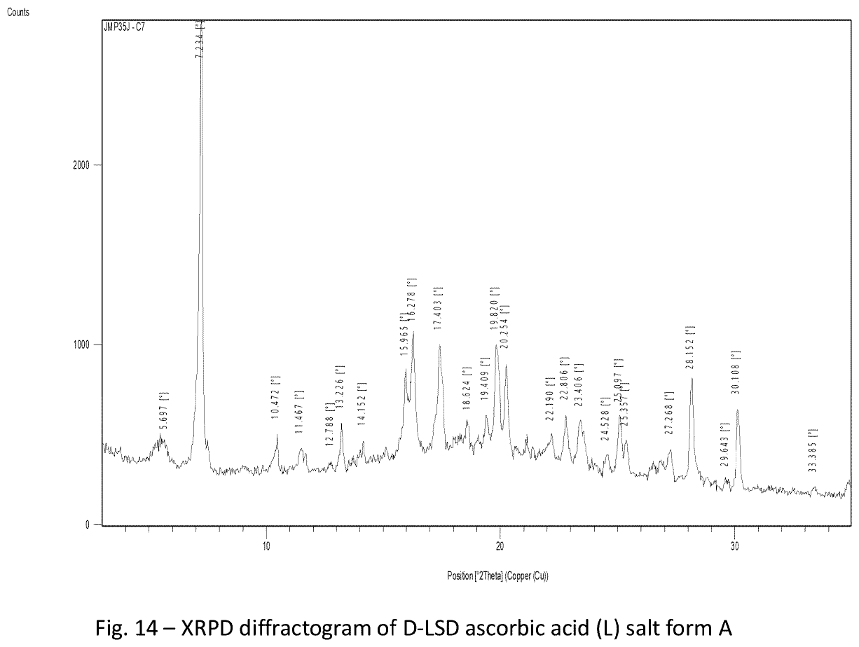

The L-ascorbic acid salt form A of D-LSD is characterized by an x-ray powder diffraction pattern obtained by irradiation with Cu Kα x-rays having peaks expressed as 2θ at about 7.2, about 16.3, about 19.8 and about 28.2 (FIG. 14).

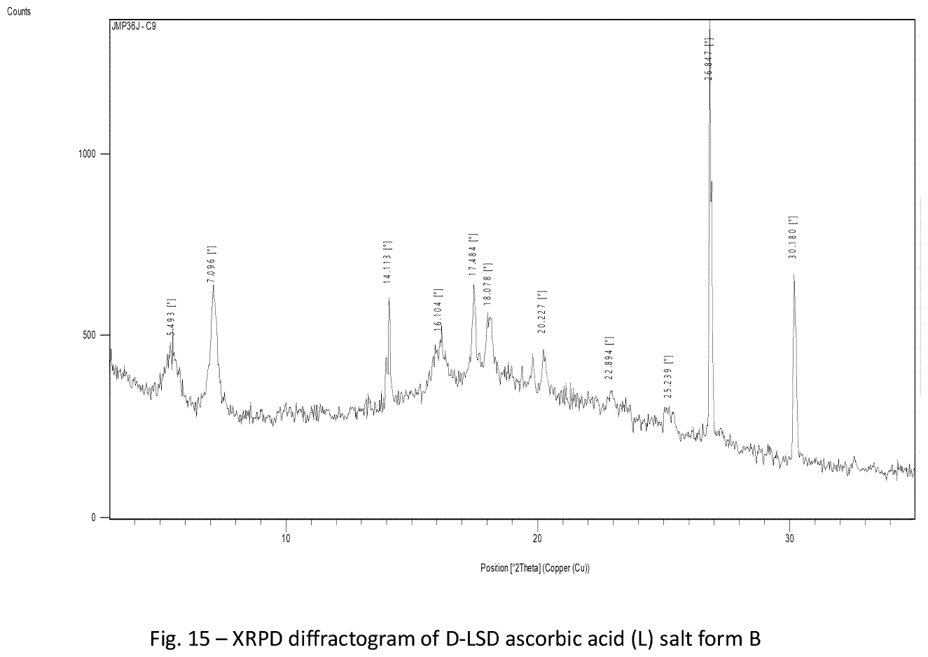

The L-ascorbic acid salt form B of D-LSD is characterized by an x-ray powder diffraction pattern obtained by irradiation with Cu Kα x-rays having peaks expressed as 2θ at about 7.1, about 17.5, about 26.8 and about 30.2 (FIG. 15).

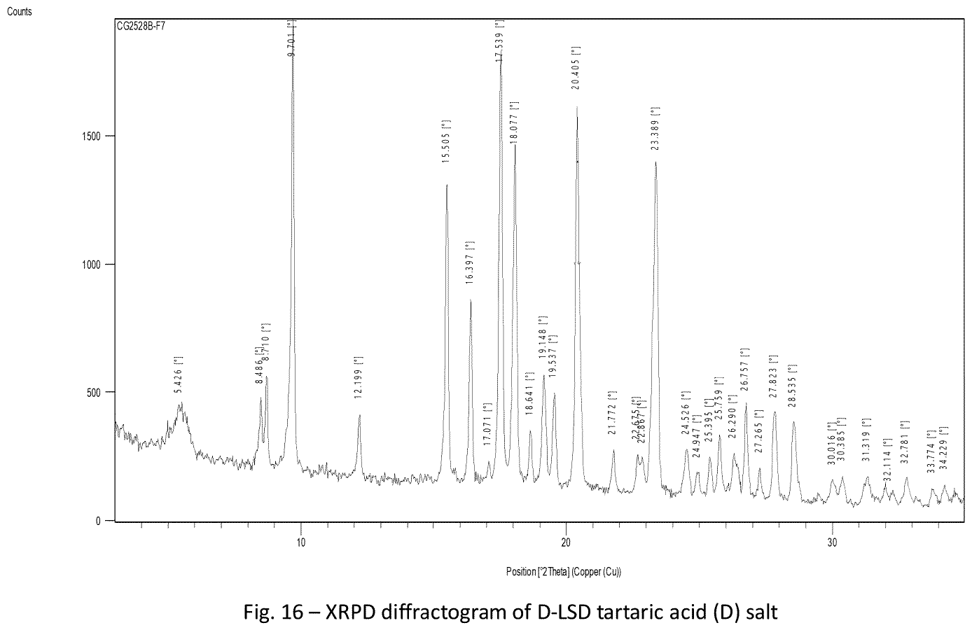

The D-tartaric acid salt of D-LSD is characterized by an x-ray powder diffraction pattern obtained by irradiation with Cu Kα x-rays having peaks expressed as 2θ at about 9.7, about 15.5, about 17.5, about 20.4 and about 23.4 (FIG. 16).

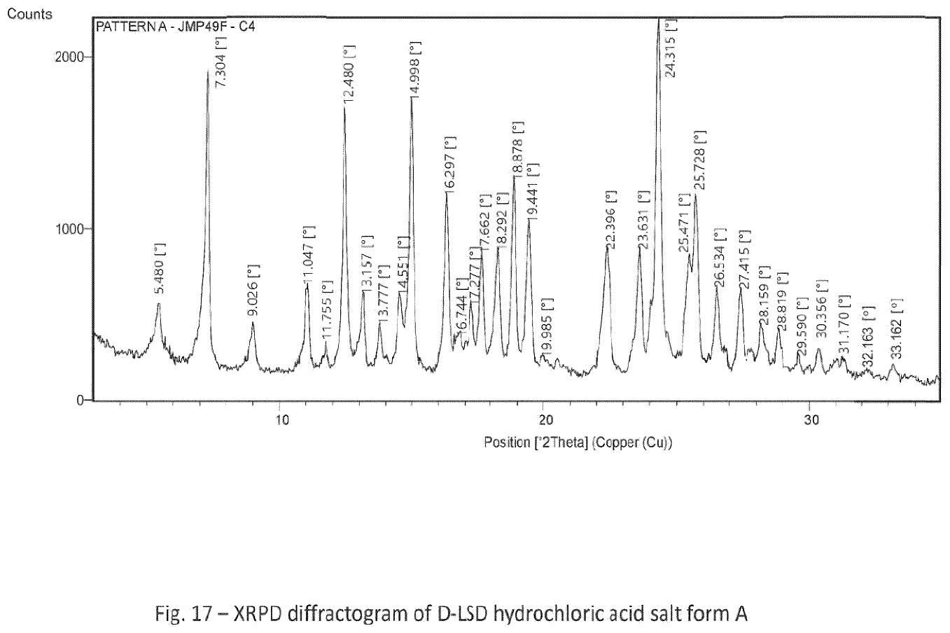

The hydrochloric acid salt form A of D-LSD is characterized by an x-ray powder diffraction pattern obtained by irradiation with Cu Kα x-rays having peaks expressed as 2θ at about 7.3, about 12.5, about 15.0, about 16.3 and about 24.3 (FIG. 17).

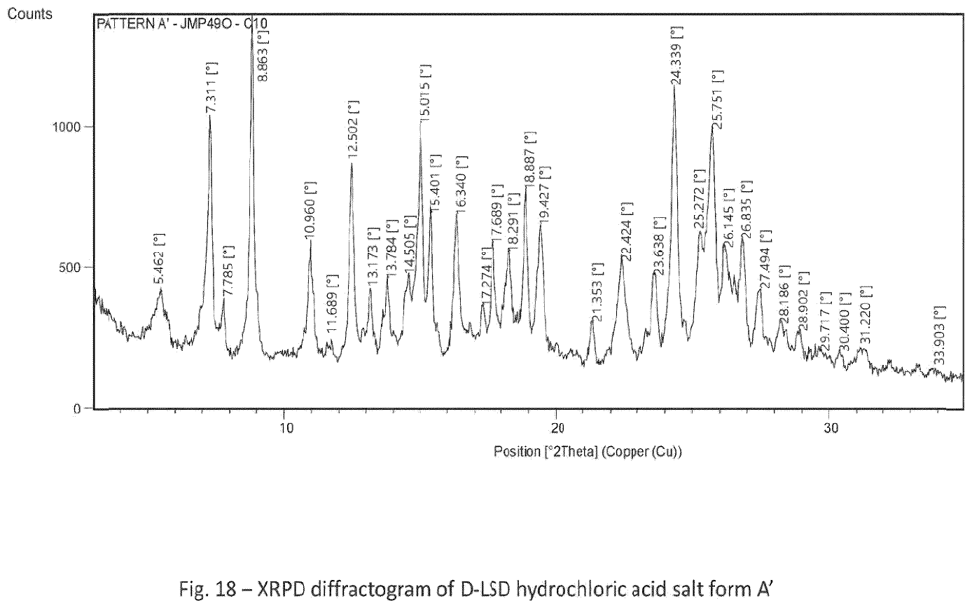

The hydrochloric acid salt form A′ of D-LSD is characterized by an x-ray powder diffraction pattern obtained by irradiation with Cu Kα x-rays having peaks expressed as 2θ at about 7.3, about 8.9, about 12.5, about 15.0 and about 24.3 (FIG. 18).

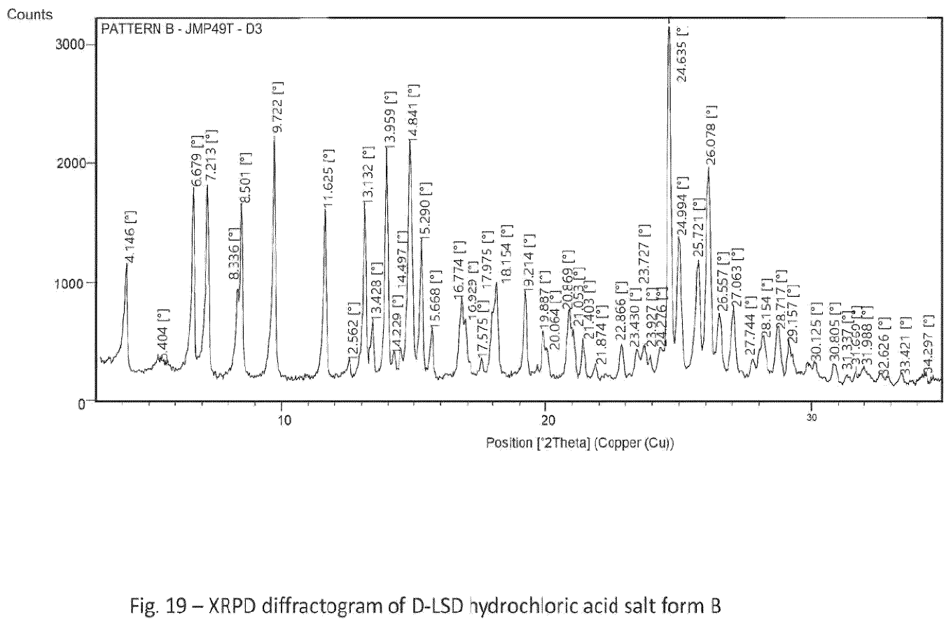

The hydrochloric acid salt form B of D-LSD is characterized by an x-ray powder diffraction pattern obtained by irradiation with Cu Kα x-rays having peaks expressed as 2θ at about 6.7, about 7.2, about 9.7, about 14.8 and about 24.6 (FIG. 19).

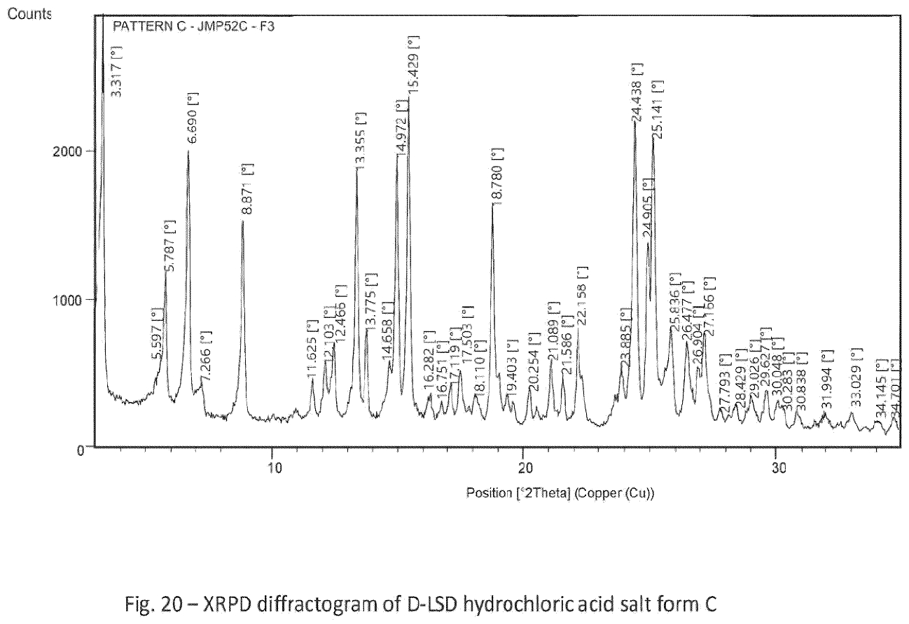

The hydrochloric acid salt form C of D-LSD is characterized by an x-ray powder diffraction pattern obtained by irradiation with Cu Kα x-rays having peaks expressed as 2θ at about 5.8, about 6.7, about 15.4, about 18.8 and about 24.4 (FIG. 20).

The hydrochloric acid salt form D of D-LSD is characterized by an x-ray powder diffraction pattern obtained by irradiation with Cu Kα x-rays having peaks expressed as 2θ at about 7.3, about 12.4, about 15.0, about 18.0 and about 24.2 (FIG. 21).

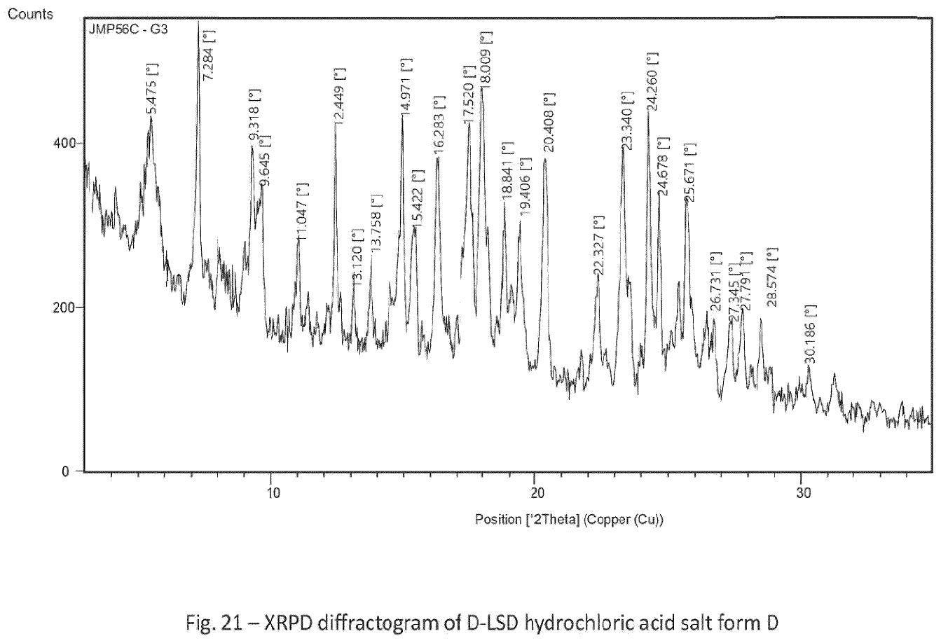

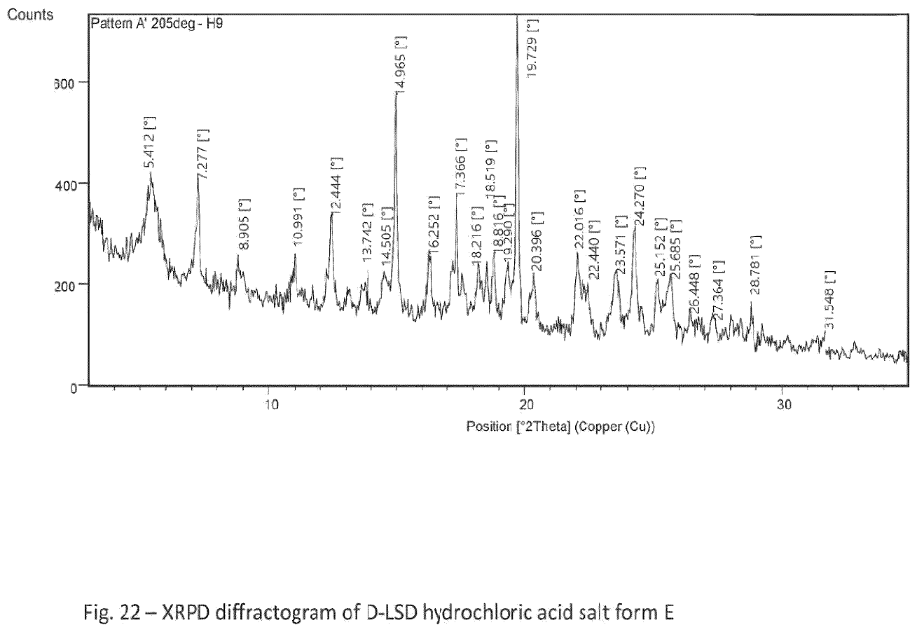

The hydrochloric acid salt form E of D-LSD is characterized by an x-ray powder diffraction pattern obtained by irradiation with Cu Kα x-rays having peaks expressed as 2θ at about 7.3, about 12.4, about 15.0, about 19.7 and about 24.3 (FIG. 22).

The LSD crystalline salt forms can be stored as is or prepared in a pharmaceutical formulation as a liquid, gel, or solid. Therefore, the present invention provides for a pharmaceutical formulation of the LSD salt crystal form including one or more pharmaceutically acceptable excipients.

The LSD crystalline forms and the LSD crystalline salt forms can have different levels of stability and dissolution rates depending on the acid used in the formulation. This can impact the quality of active pharmaceutical ingredients, conversion to desired formulations, and performance/absorption rates when delivered orally.

In one embodiment, the polymorphic form of LSD is formulated as a pharmaceutical composition for oral, iv, sublingual, topical, transdermal, subcutaneous, intramuscular, intranasal, or rectal administration. In some embodiments, the polymorphic form of LSD is formulated for oral administration. In some embodiments, the polymorphic form of LSD is formulated for iv administration. In some embodiments, the polymorphic form of LSD is formulated for sublingual administration. In some embodiments, the polymorphic form of LSD is formulated for topical administration. In some embodiments, the polymorphic form of LSD is formulated for transdermal administration. In some embodiments, the polymorphic form of LSD is formulated for subcutaneous administration. In some embodiments, the polymorphic form of LSD is formulated for intramuscular administration. In some embodiments, the polymorphic form of LSD is formulated for rectal administration.

In one embodiment, the polymorphic form of a salt form of LSD is formulated as a pharmaceutical composition for oral, iv, sublingual, topical, transdermal, subcutaneous, intramuscular, or rectal administration. In some embodiments, the polymorphic form of a salt form of LSD is formulated for oral administration. In some embodiments, the polymorphic form of a salt form of LSD is formulated for iv administration. In some embodiments, the polymorphic form of a salt form of LSD is formulated for sublingual administration. In some embodiments, the polymorphic form of a salt form of LSD is formulated for topical administration. In some embodiments, the polymorphic form of a salt form of LSD is formulated for transdermal administration. In some embodiments, the polymorphic form of a salt form of LSD is formulated for subcutaneous administration. In some embodiments, the polymorphic form of a salt form of LSD is formulated for intramuscular administration. In some embodiments, the polymorphic form of a salt form of LSD is formulated for rectal administration.

The compositions of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In one embodiment, the compositions of the present invention are administered to deliver from about 1 μg to about 1 mg of LSD free-base equivalent per dosage to a subject in need of treatment. In some embodiments, the compositions of the present invention are administered to deliver from about 1 μg to about 10 μg of LSD free-base equivalent per dosage to a subject in need of treatment. In some embodiments, the compositions of the present invention are administered to deliver from about 3 μg to about 6 μg of LSD free-base equivalent per dosage to a subject in need of treatment. In some embodiments, the compositions of the present invention are administered to deliver from about 5 μg to about 20 μg of LSD free-base equivalent per dosage to a subject in need of treatment. In some embodiments, the compositions of the present invention are administered to deliver from about 15 μg to about 50 μg of LSD free-base equivalent per dosage to a subject in need of treatment. In some embodiments, the compositions of the present invention are administered to deliver from about 40 μg to about 100 μg of LSD free-base equivalent per dosage to a subject in need of treatment. In some embodiments, the compositions of the present invention are administered to deliver from about 100 μg to about 1 mg of LSD free-base equivalent per dosage to a subject in need of treatment. In the method of the present invention, the compounds of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, subcutaneously, or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

In one embodiment, the compositions of the present invention are administered to a human subject from one to four times per day. In some embodiments, the compositions of the present invention are administered to a human subject once per day. In some embodiments, the compositions of the present invention are administered to a human subject twice per day. In some embodiments, the compositions of the present invention are administered to a human subject three times per day. In some embodiments, the compositions of the present invention are administered to a human subject four times per day.

In one embodiment, the duration of treating a human subject is daily, daily for 7 days, daily for 14 days or daily for 30 days. In some embodiments, the duration of treating a human subject is daily. In some embodiments, the duration of treating a human subject is daily for 7 days. In some embodiments, the duration of treating a human subject is daily for 14 days. In some embodiments, the duration of treating a human subject is daily for 30 days.

When administering a composition of the present invention orally, it will generally be formulated in a unit dosage form selected from the list comprising solution, suspension, emulsion, tablet, and capsule.

When administering a composition of the present invention sublingually, it will generally be formulated in a unit dosage form selected from the list comprising tablet and strip wherein said tablet and strip are rapidly dissolving.

When administering a composition of the present invention topically, it will generally be formulated in a unit dosage form selected from the list comprising cream and ointment.

When administering a composition of the present invention transdermally, it will generally be formulated in a unit dosage form comprising a patch.

When administering a composition of the present invention rectally, it will generally be formulated in a unit dosage form selected from the list comprising a suppository.

When administering a composition of the present invention intranasally, it will generally be formulated in a dosage form selected from the list comprising a dry powder, a solution, and an emulsion. One skilled in the art will recognize that intranasal administration generally requires a delivery device calibrated for delivery of a specify dosage per use.

When administering a composition of the present invention by injection (iv, subcutaneous, intramuscular), it will generally be formulated in a unit dosage form selected from the list comprising solution, suspension, and emulsion. The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Sterile injectable solutions can be prepared by incorporating the compositions utilized in practicing the present invention in the required amount of the appropriate solvent with various of the other ingredients, as desired.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1—General Preparation of D-LSD Salt Forms

A solution of an acid dissolved in ethanol was combined with a solution of D-LSD free-base in tetrahydrofuran. Precipitated materials were collected by filtration and subsequently subjected to crystallization studies to generate crystalline forms. If solid forms were not isolated, alternate solvents including dichloromethane, ethyl acetate, acetone and acetonitrile were evaluated.

### Example 2—General Preparation of D-LSD Salt Form Crystal Forms

Solid salt forms of D-LSD were subjected to standard crystallization methods including solvent-mediated equilibration, antisolvent-mediated crystallization, mixed-solvent recrystallization, solvent-vapor diffusion crystallization, compression and grinding of solid salt forms. Isolated solid salt forms were evaluated by DVS, DSC, TGA, and XRPD to characterize the crystalline forms.

### Example 3-Naphthalene-2-Sulfonate Salt

A crystalline form of a D-LSD salt with naphthalene-2-sulfonic acid was prepared as described according to Example 1 and Example 2. The crystalline form was characterized by an x-ray powder diffraction pattern obtained by irradiation with Cu Kα x-rays having peaks expressed as 2θ at about 5.0, about 6.1, about 11.5, about 17.3 and about 18.9.

### Example 4-L-Tartarate Hemi-Salt

A crystalline form of a D-LSD hemi-salt with L-tartaric acid was prepared as described according to Example 1 and Example 2. The crystalline form was characterized by an x-ray powder diffraction pattern obtained by irradiation with Cu Kα x-rays having peaks expressed as 2θ at about 9.6, about 11, about 20.8 and about 24.7.

### Example 5-Maleate Salt Form A

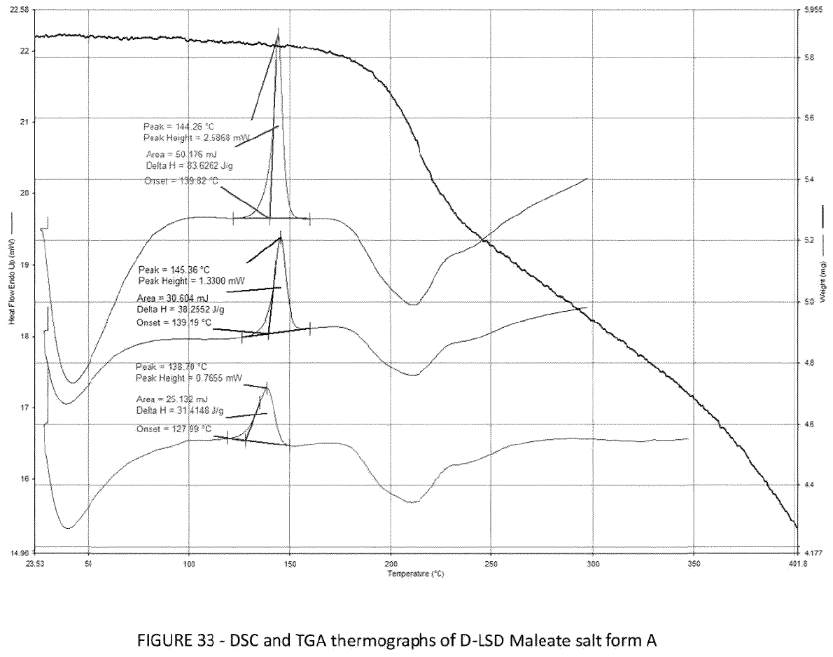

A crystalline form of a D-LSD salt with maleic acid was prepared as described according to Example 1 and Example 2. The crystalline form was characterized by an x-ray powder diffraction pattern obtained by irradiation with Cu Kα x-rays having peaks expressed as 2θ at about 8.8, about 16.2, about 17.4, about 18.8, about 20.1 and about 25.0 (FIG. 3). The crystalline form was characterized by DSC and TGA (FIG. 33) The DSC thermograph of the solid shows a main melt endotherm at 145° C.

### Example 6-Maleate Salt Form B

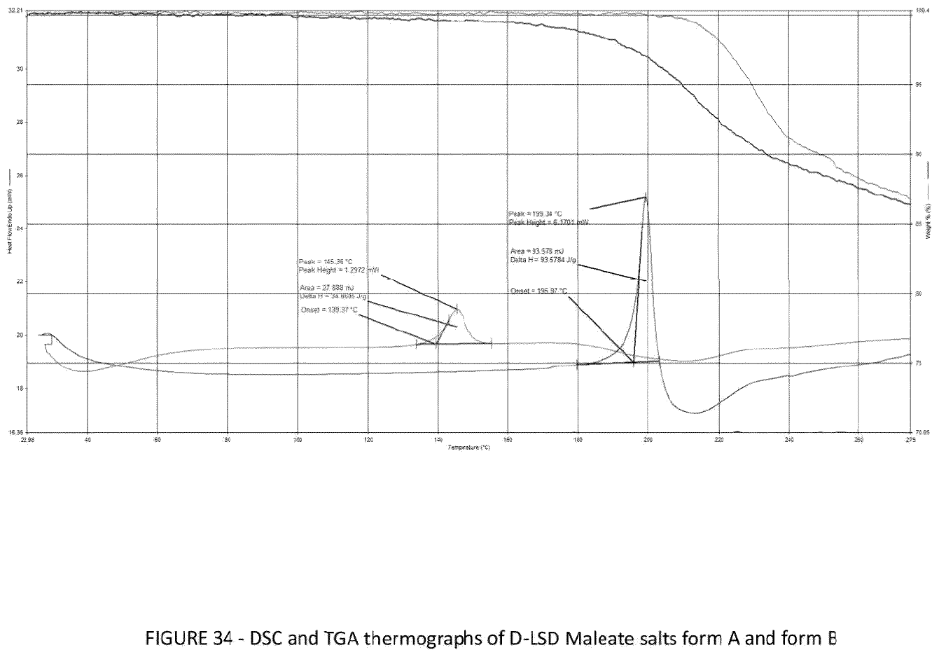

A crystalline form of a D-LSD salt with maleic acid was prepared as described according to Example 1 and Example 2. The crystalline form was characterized by an x-ray powder diffraction pattern obtained by irradiation with Cu Kα x-rays having peaks expressed as 2θ at about 8.4, about 14.7, about 15.1, about 17.4 and about 22.1 (FIG. 4). The crystalline form was characterized by DSC and TGA (FIG. 34). The DSC thermograph of the solid shows a main melt endotherm at 199° C. This is the preferred maleate.

### Example 7-Xinafoate Salt Form A

A crystalline form of LSD was prepared as described by methods above. The acid was xinafoic acid, and the crystalline form was characterized by an x-ray powder diffraction pattern obtained by irradiation with Cu Kα x-rays having peaks expressed as 2θ at about 10.2, about 11.6, about 13.3, about 15.6 and about 21.6.

### Example 8-Xinafoate Salt Form A′

A crystalline form of LSD was prepared as described by methods above. The acid was xinafoic acid, and the crystalline form was characterized by an x-ray powder diffraction pattern obtained by irradiation with Cu Kα x-rays having peaks expressed as 2θ at about 14.8, about 15.6, about 21.5 and about 26.6.

### Example 9-Phosphate Salt Form A

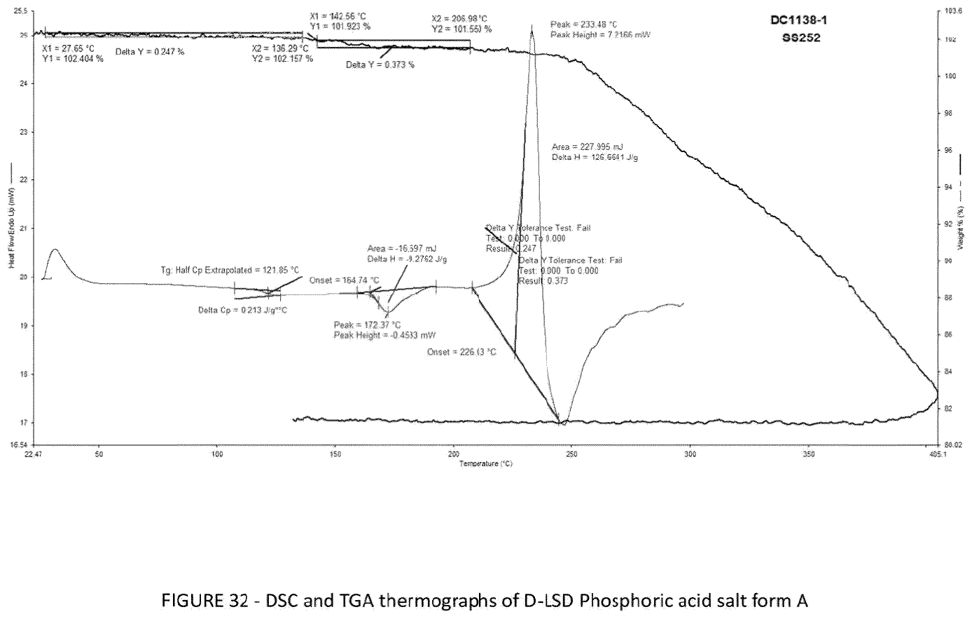

A crystalline form of LSD was prepared as described by methods above. The acid was phosphoric acid, and the crystalline form was characterized by an x-ray powder diffraction pattern obtained by irradiation with Cu Kα x-rays having peaks expressed as 2θ at about 7.2, about 13.5, about 14.8, about 16.9, about 24.2 and about 24.5. FIG. 7 is an XRPD Pattern of LSD phosphate Pattern A. The material was characterized by DSC and TGA (FIG. 32). DSC/TGA analysis showed the Tg at 122° C. (half Cp) followed by an exothermic event at 165° C. and the melting endotherm at 233° C. A weight loss of 0.2% w/w was observed between 27-136° C., followed by a second minimal weight loss of 0.37% w/w.

### Example 10-Naphthalene-1,5-Disulfonate Hemi-Salt

A crystalline form of LSD was prepared as described by methods above. The acid was naphthalene-1,5-disulfonic acid, and the crystalline form was characterized by an x-ray powder diffraction pattern obtained by irradiation with Cu Kα x-rays having peaks expressed as 2θ at about 10.1, about 14.4, about 16.7, about 23.5, about 24.5, about 21.9 and about 22.6.

### Example 11-Oxalate Salt Form A

A crystalline form of LSD was prepared as described by methods above. The acid was oxalic acid, and the crystalline form was characterized by an x-ray powder diffraction pattern obtained by irradiation with Cu Kα x-rays having peaks expressed as 2θ at about 8.2, about 10.4, about 17.7, about 19.2 and about 21.9.

### Example 12-Benzoate Salt Form A

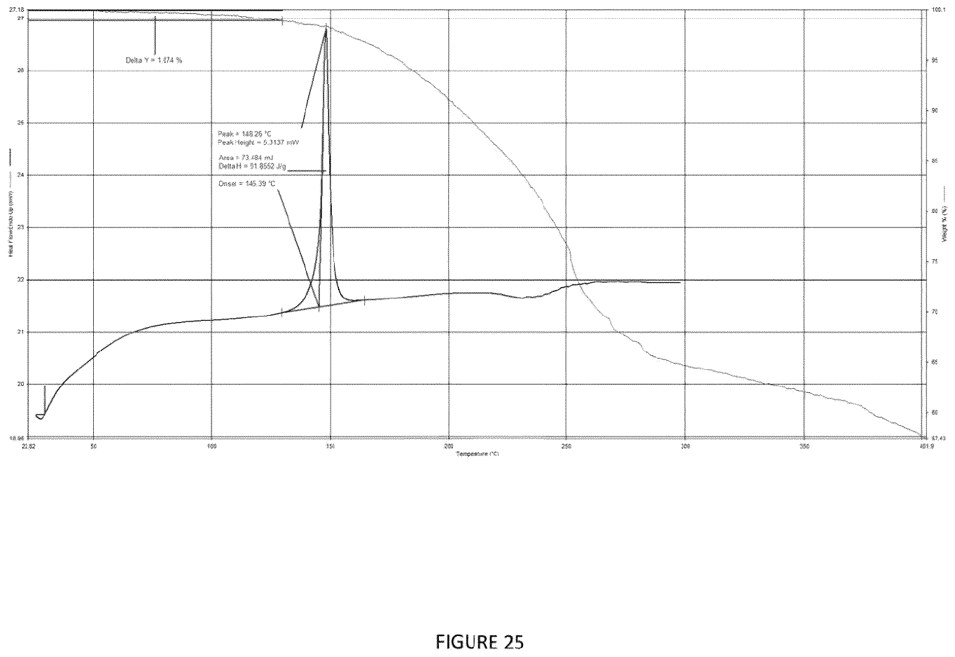

A crystalline form of a D-LSD salt with benzoic acid was prepared as described according to Example 1 and Example 2. The crystalline form was characterized by an x-ray powder diffraction pattern obtained by irradiation with Cu Kα x-rays having peaks expressed as 2θ at about 8.5, about 14.6, about 16.6, about 18.5 and about 19.0 (FIG. 10). The material was characterized by DSC and TGA (FIG. 25). DSC showed an endothermic peak at 148° C. TGA showed a 1.0% loss of mass between 25° C. and 130° C. This is the preferred benzoate form.

### Example 13-Benzoate Salt Form B

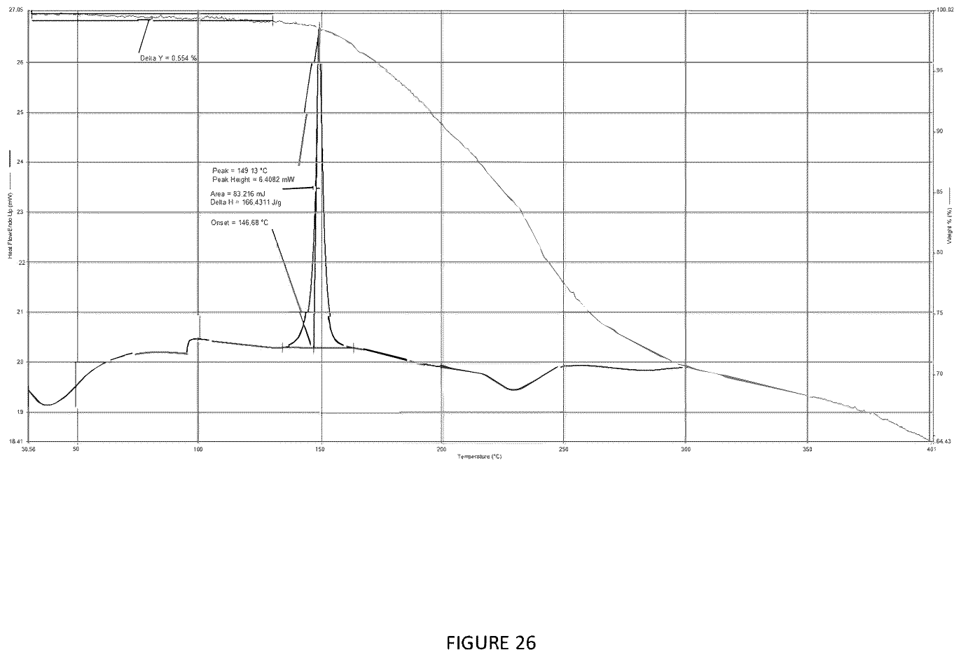

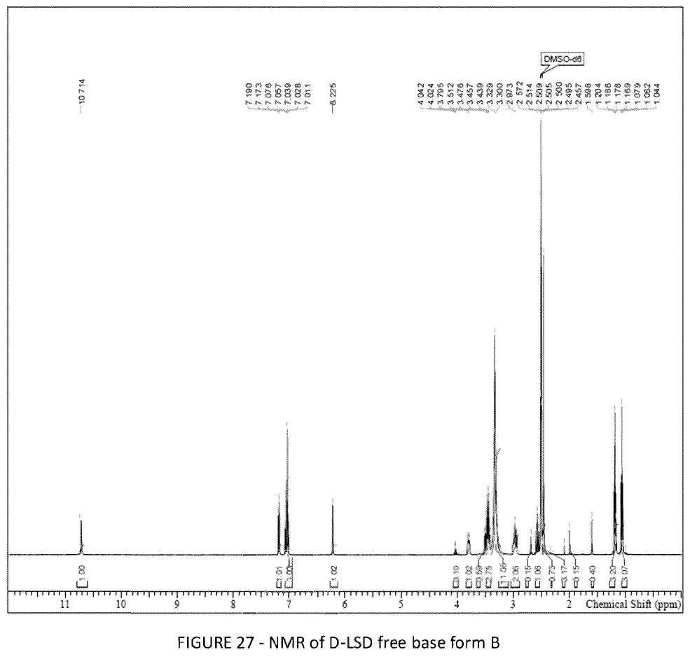

A crystalline form of a D-LSD salt with benzoic acid was prepared as described according to Example 1 and Example 2. The crystalline form was characterized by an x-ray powder diffraction pattern obtained by irradiation with Cu Kα x-rays having peaks expressed as 2θ at about 17.3, about 19.8, about 20.4 and about 25.2 (FIG. 11). The material was characterized by DSC and TGA (FIG. 26). DSC showed an endothermic peak at 149° C. TGA showed a 0.6% loss of mass between 25° C. and 130° C.

### Example 14-Fumarate Salt

A crystalline form of LSD was prepared as described by methods above. The acid was fumaric acid, and the crystalline form was characterized by an x-ray powder diffraction pattern obtained by irradiation with Cu Kα x-rays having peaks expressed as 2θ at about 11.2, about 18.4, about 19.7 and about 22.1 (FIG. 12).

### Example 15-Fumarate Hemi-Salt

A crystalline form of LSD was prepared as described by methods above. The acid was fumaric acid, and the crystalline form was characterized by an x-ray powder diffraction pattern obtained by irradiation with Cu Kα x-rays having peaks expressed as 2θ at about 7.5, about 21.3, about 22.7 and about 24.7 (FIG. 12).

### Example 16-L-Ascorbate Salt Form A

A crystalline form of LSD was prepared as described by methods above. The acid was L-ascorbic acid, and the crystalline form was characterized by an x-ray powder diffraction pattern obtained by irradiation with Cu Kα x-rays having peaks expressed as 2θ at about 7.2, about 16.3, about 19.8 and about 28.2 (FIG. 14).

### Example 17-L-Ascorbate Salt Form B

A crystalline form of LSD was prepared as described by methods above. The acid was L-ascorbic acid, and the crystalline form was characterized by an x-ray powder diffraction pattern obtained by irradiation with Cu Kα x-rays having peaks expressed as 2θ at about 7.1, about 17.5, about 26.8 and about 30.2 (FIG. 15).

### Example 18-D-Tartrate Salt

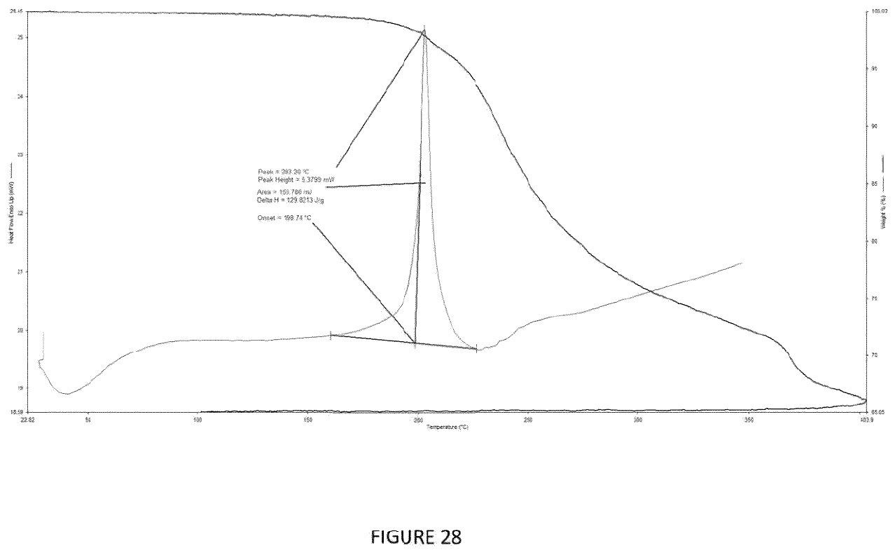

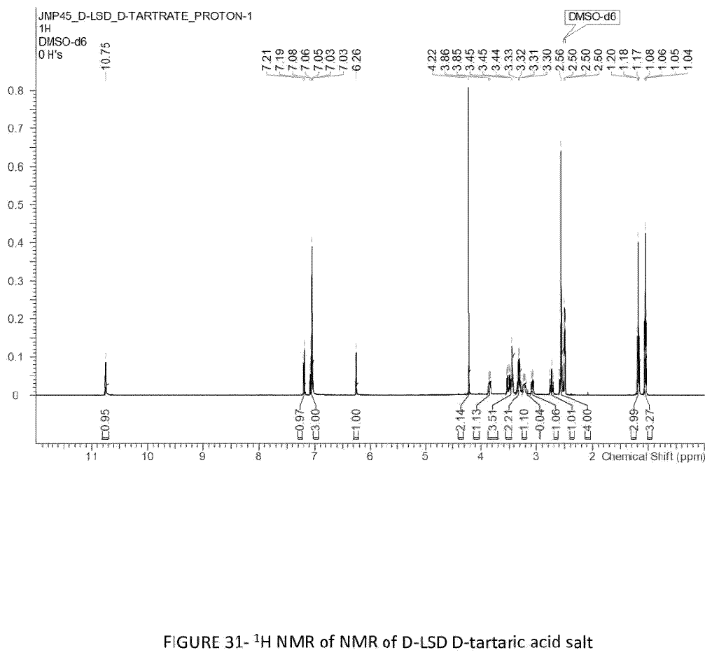

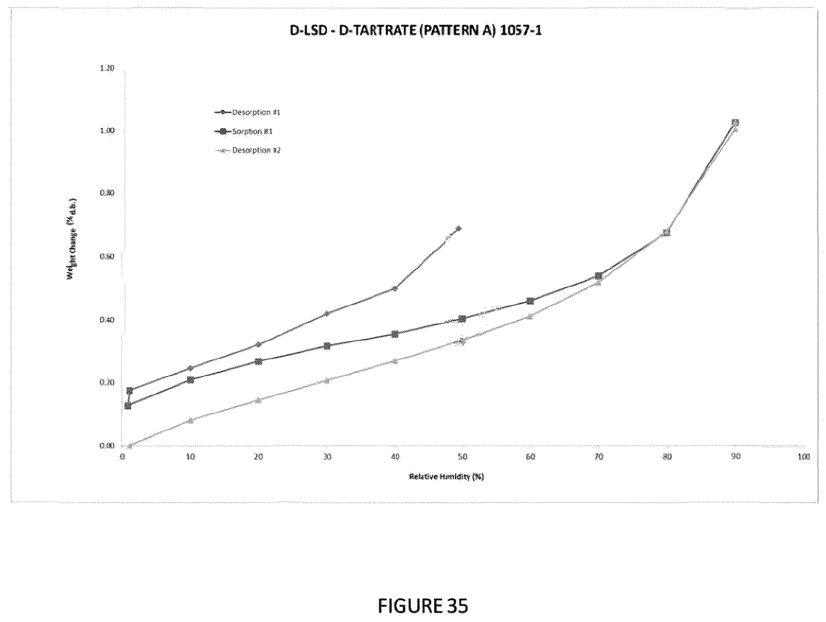

A crystalline form of a D-LSD salt with D-tartaric acid was prepared as described according to Example 1 and Example 2. The crystalline form was characterized by XRPD, DSC, TGA, and DVS. The x-ray powder diffraction pattern obtained by irradiation with Cu Kα x-rays having peaks expressed as 2θ at about 9.7, about 15.5, about 17.5, about 20.4 and about 23.4 (FIG. 16). The material was characterized by DSC and TGA (FIG. 28). The DSC thermograph contains a main melt endotherm at 203° C. and the corresponding TGA thermograph shows no weight reduction until decomposition. The ¹H NMR spectrum (FIG. 31) confirms mono stoichiometry of the salt with a tartrate peak at 4.2 ppm (2H). The DVS profile of D-LSD D-tartrate Pattern A is shown in FIG. 35. During the initial desorption cycle the solid lost 0.6 wt % from 50% RH to 0% RH and did not achieve an anhydrous state. During the subsequent sorption cycle the solid absorbed 0.65% of water up to 80% RH. This increased to 1% at 90% RH. The DVS profile confirms that this water uptake is reversible as relative humidity decreases with only minor hysteresis indicated. Solvent exclusion is suggested as the cycle progressed, leading to a dry solid upon completion of the final desorption.

### Example 19-Hydrochloride Salt Form A

A crystalline form of a D-LSD salt with hydrochloric acid was prepared as described according to Example 1 and Example 2. The crystalline form was characterized by an x-ray powder diffraction pattern obtained by irradiation with Cu Kα x-rays having peaks expressed as 2θ at about 7.3, about 12.5, about 15.0, about 16.3 and about 24.3 (FIG. 17).

### Example 20-Hydrochloride Salt Form A′

A crystalline form of a D-LSD salt with hydrochloric acid was prepared as described according to Example 1 and Example 2. The crystalline form was characterized by an x-ray powder diffraction pattern obtained by irradiation with Cu Kα x-rays having peaks expressed as 2θ at about 7.3, about 8.9, about 12.5, about 15.0 and about 24.3 (FIG. 18).

### Example 21-Hydrochloride Salt Form B

A crystalline form of a D-LSD salt with hydrochloric acid was prepared as described according to Example 1 and Example 2. The crystalline form was characterized by an x-ray powder diffraction pattern obtained by irradiation with Cu Kα x-rays having peaks expressed as 2θ at about 6.7, about 7.2, about 9.7, about 14.8 and about 24.6 (FIG. 19).

### Example 22-Hydrochloride Salt Form C

A crystalline form of a D-LSD salt with hydrochloric acid was prepared as described according to Example 1 and Example 2. The crystalline form was characterized by an x-ray powder diffraction pattern obtained by irradiation with Cu Kα x-rays having peaks expressed as 2θ at about 5.8, about 6.7, about 15.4, about 18.8 and about 24.4 (FIG. 20).

### Example 23-Hydrochloride Salt Form D

A crystalline form of a D-LSD salt with hydrochloric acid was prepared as described according to Example 1 and Example 2. The crystalline form was characterized by an x-ray powder diffraction pattern obtained by irradiation with Cu Kα x-rays having peaks expressed as 2θ at about 7.3, about 12.4, about 15.0, about 18.0 and about 24.2 (FIG. 21).

### Example 24-Hydrochloride Salt Form E

A crystalline form of a D-LSD salt with hydrochloric acid was prepared as described according to Example 1 and Example 2. The crystalline form was characterized by an x-ray powder diffraction pattern obtained by irradiation with Cu Kα x-rays having peaks expressed as 2θ at about 7.3, about 12.4, about 15.0, about 19.7 and about 24.3 (FIG. 22).

### Example 25-Free-Base Form A

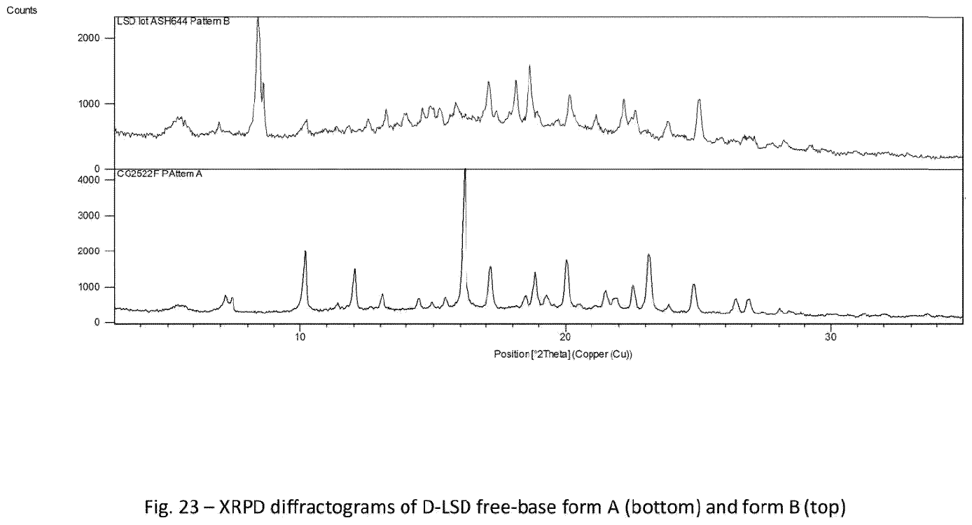

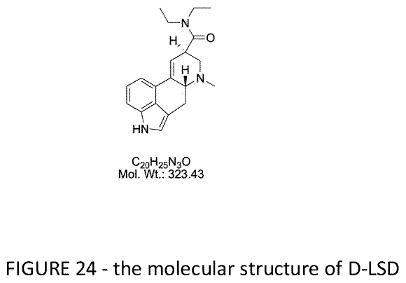

A crystalline form of D-LSD free-base was prepared by crystallization from ethyl acetate. The crystalline form was characterized by an x-ray powder diffraction pattern obtained by irradiation with Cu Kα x-rays having peaks expressed as 2θ at about 10.2, about 12.0, about 16.2, about 17.2, about 20.0 and about 23.1 (FIG. 23, bottom).

### Example 26-Free-Base Form B

A crystalline form of D-LSD free-base was prepared by a workup that incorporated a 1M NaOH wash. The crystalline form was characterized by an x-ray powder diffraction pattern obtained by irradiation with Cu Kα x-rays having peaks expressed as 2θ at about 8.5, about 8.6, about 17.1, about 18.1, about 18.7 and about 25.0. Thermal analysis was collected for the Pattern B solid. The DSC thermograph contains a first endotherm with an onset at 71° C. and a second endotherm at 108° C. The corresponding TGA thermograph showed a weight loss of 4.12% w/w between 32-222° C. (FIG. 23, top).

### Example 27-Phosphate Form B

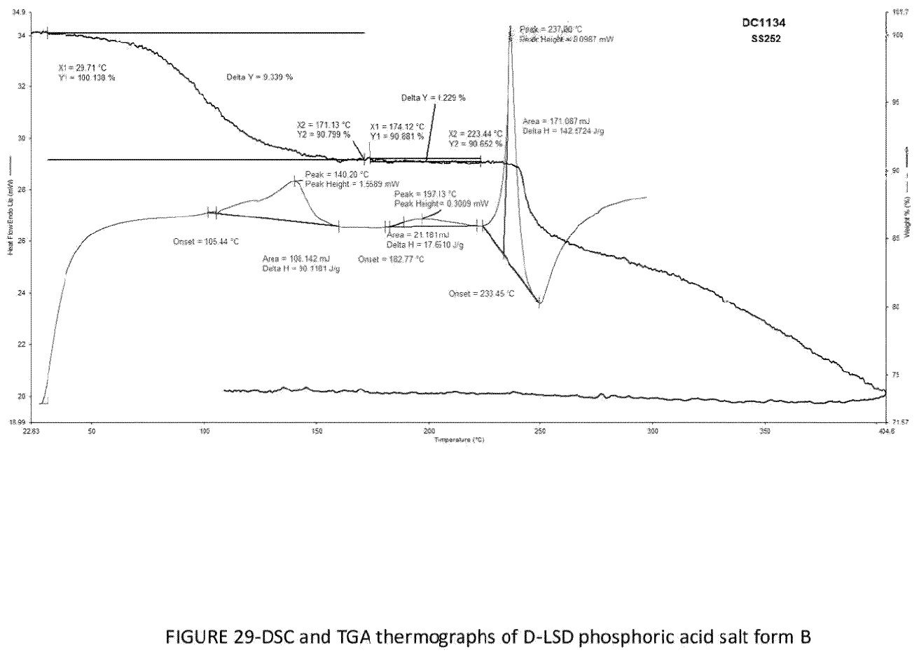

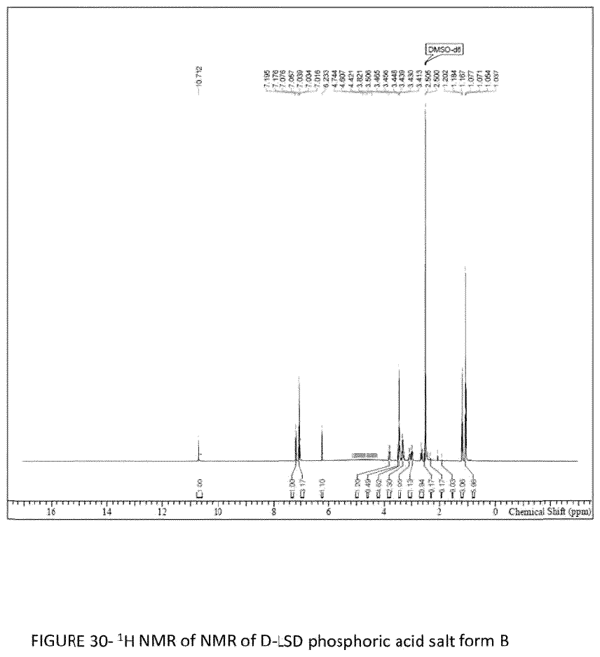

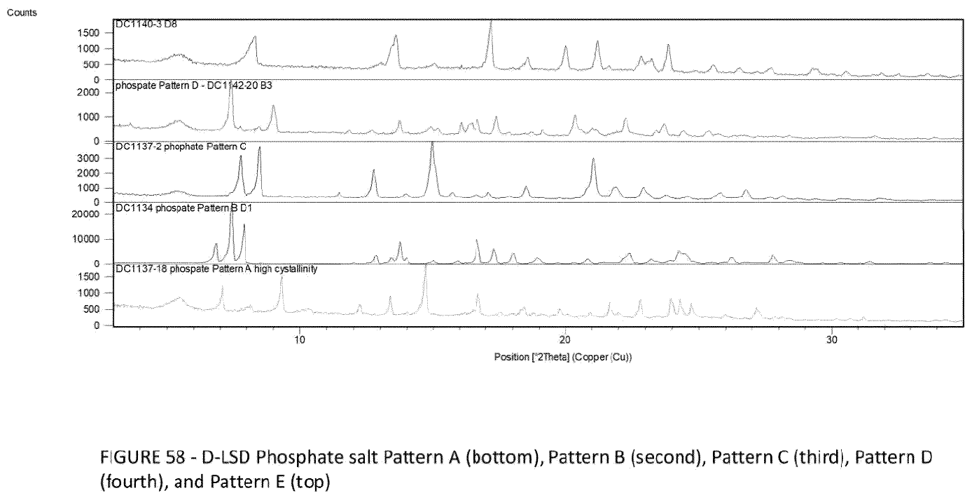

D-LSD Free Base (Pattern A, 2.02 g) was placed in a round bottom flask (V=50 mL) and dissolved in EtOH (10 V). The solution was heated to 50° C., at 300 rpm for 30 minutes. Then, a stock solution of phosphoric acid prepared in EtOH (1 M, 6.25 mL) was slowly added. The system was equilibrated for 15 minutes, and the temperature was decreased to 25° C. overnight. A suspension was observed and was filtered to isolate the solid. The recovered solid was placed in a vacuum oven at 45° C. for 48 hours. Yield 82%. The material was analyzed by XRPD, DSC/TGA and proton NMR. The crystalline form was characterized by an x-ray powder diffraction pattern obtained by irradiation with Cu Kα x-rays having peaks expressed as about 6.8, about 7.5, about 7.9, about 13.8, about 16.7 and about 17.3 (FIG. 58, second from bottom). Pattern B form of D-LSD phosphate salt was characterized by DSC/TGA as a crystalline mono ethanol solvate solid with a melt temperature of 223° C. (FIG. 29). DSC/TGA thermograms showed a first endo onset at 105° C., a second endo at 182° C. followed by the melting endo at 233° C. This main melt corresponded with that of Pattern A. A weight loss of 9.4% w/w (~1 mol eq. of EtOH) between 29-171° C. was present in the TGA thermogram. This was by followed a small weight loss of 0.3% w/w between 174-223° C.

### Example 28-Phosphate Form C

An alternate crystalline form of LSD phosphate was prepared via heat cycled maturation from LSD phosphate form B as shown in TABLE 1.

TABLE 1

| | | | | Solubility |
|---|---|---|---|---|
| | | Form fate by XRPD | | 25° C. |
| ID | Solvent | 25° C. | 45° C./70° C. | (mg/ml) |
| 1 | EtOH | Pattern A + | Pattern C | 2.36 |
| | | Pattern C | | |
| 2 | MeOH | Pattern C | Pattern C | — |
| 3 | TBME | Pattern C + | Pattern C | — |
| | | peaks | | |
| 4 | THF | Pattern C | Pattern C | 2.01 |
| 5 | Me—THF | Pattern C | Pattern C | 0.35 |
| 6 | CPME | Pattern C | Pattern C | — |
| 7 | 1,4-dioxane | Pattern C | Pattern C | 4.57 |
| 8 | EtOAc | Pattern C | Pattern C | 0.11 |
| 9 | IPAC | Pattern C | Pattern C | 0.05 |
| 10 | acetone | Pattern C | Pattern C | — |
| 11 | MEK | Pattern C | Pattern C | 0.2 |
| 12 | MIBK | Pattern C | Pattern C | 0.20 |
| 13 | Anisole | Pattern C | Pattern C | — |
| 14 | toluene | Pattern C | Pattern C + | 1.83 |
| | | | peaks | |
| 15 | Chlorobenzene | Pattern C | Amorphous + | — |
| | | | peaks | |
| 16 | MeCN | Pattern C | Pattern C | 0.24 |
| 17 | Benzotrifluoride | Pattern C | Pattern C | 0.15 |
| 18 | heptane | Pattern A | Pattern A − | — |
| | | | high intensity | |
| | | | peaks | |
| 19 | cyclohexane | Pattern A | amorphous + | — |
| | | | Pattern C | |
| 20 | nitromethane | Pattern C | Pattern C | 0.34 |
| 21 | DCM | Pattern C | Pattern C | — |
| 22 | THF: | Pattern C | Pattern C | 5.66 |
| | water (95:5) | | | |
| 23 | iPrOAc: | Pattern C | Pattern C | 0.11 |
| | water (98:2) | | | |
| 24 | IPA: | Pattern B + | Pattern B + | 0.40 |
| | water (95:5) | peaks | peaks | |

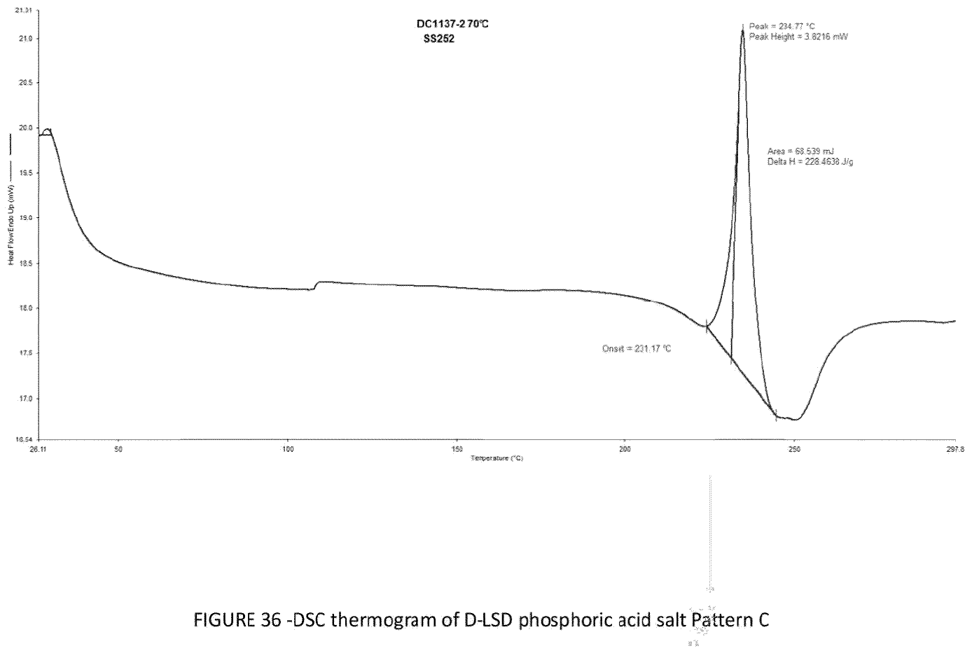

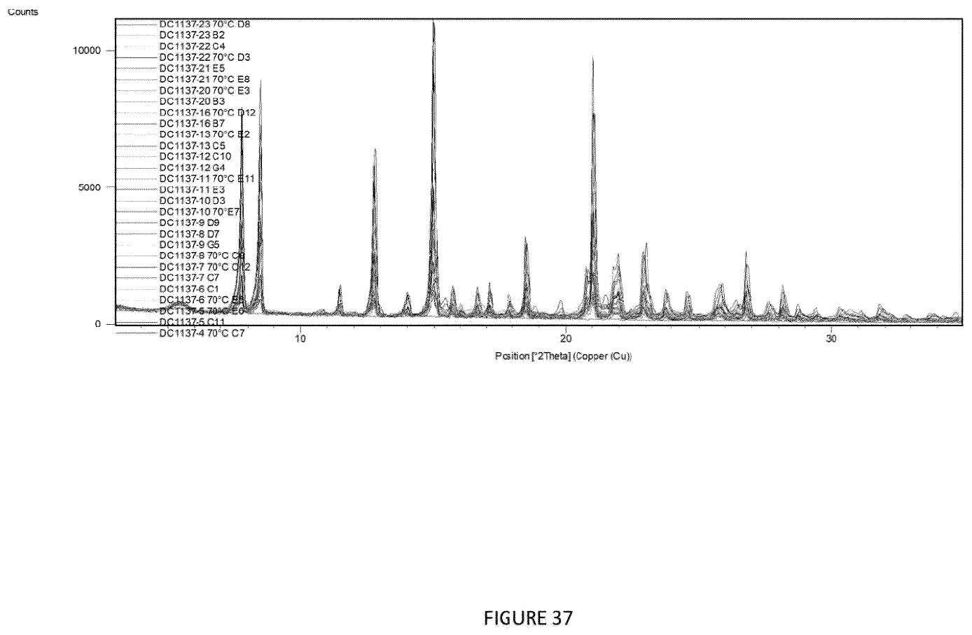

The crystalline form was characterized by an x-ray powder diffraction pattern obtained by irradiation with Cu Kα x-rays having peaks expressed as about 7.8, about 8.5, about 12.8, about 15.0, about 15.1 and about 21.1. This is the preferred phosphate form. FIG. 36 is a DSC thermogram of LSD phosphate Pattern C. FIG. 37 is an XRPD Pattern of LSD phosphate Pattern C.

### Example 29-Oxalate Form B

A crystalline form of LSD was prepared as described by methods above. The acid was phosphoric acid, and the crystalline form was characterized by an x-ray powder diffraction pattern obtained by irradiation with Cu Kα x-rays having peaks expressed as about 13.0, about 15.4, about 17.9, about 19.8, about 21.5 and about 23.1. This is the preferred oxalate form.

### Example 30-Hydrobromide Form A

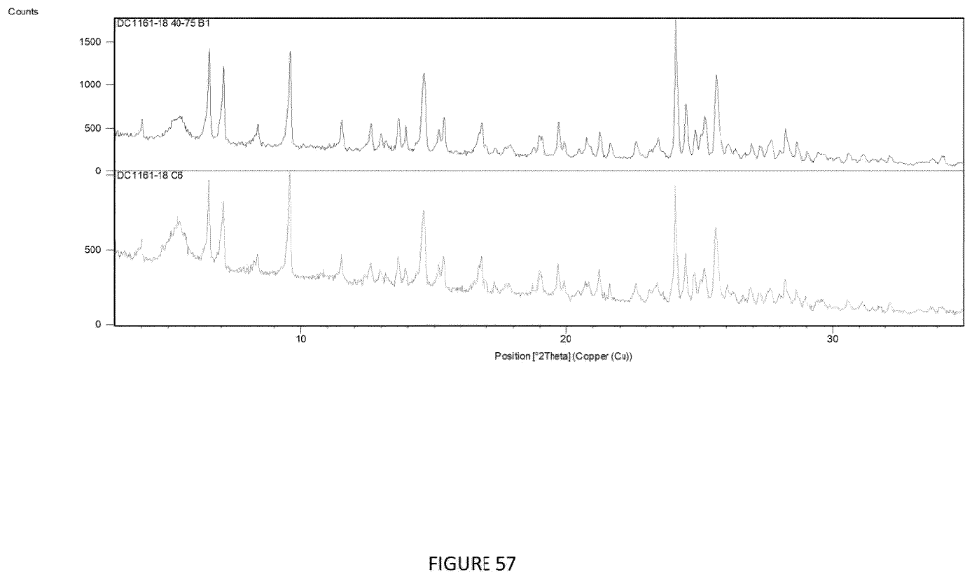

A crystalline form of LSD was prepared as described by methods above. The acid was hydrobromic acid, and the crystalline form was characterized by an x-ray powder diffraction pattern obtained by irradiation with Cu Kα x-rays having peaks expressed as about 6.6, about 9.6, about 14.7, about 24.2, about 24.5 and about 25.6. FIG. 57 is an XRPD pattern of D-LSD Hydrobromide salt Pattern A starting material (bottom) and after stress at 40° C./75 RH (top).

### Example 31-Amorphous Oxalate Salt

D-LSD Free Base (Pattern A, 1.97 g) was placed in a round bottom flask (V=50 mL) and dissolved in EtOH (10 V). The solution was heated to 50° C., at 300 rpm for 30 min. Then, a stock solution of oxalic acid prepared in EtOH (1 M, 6.03 mL) was slowly added. The system was equilibrated for 15 minutes, and the temperature was decreased to 25° C. overnight. A solution was observed and was dried under vacuum using a rotavapor. The material was weighed, and the yield was 98%.

The powder recovered was analyzed by XRPD, DSC/TGA and proton NMR.

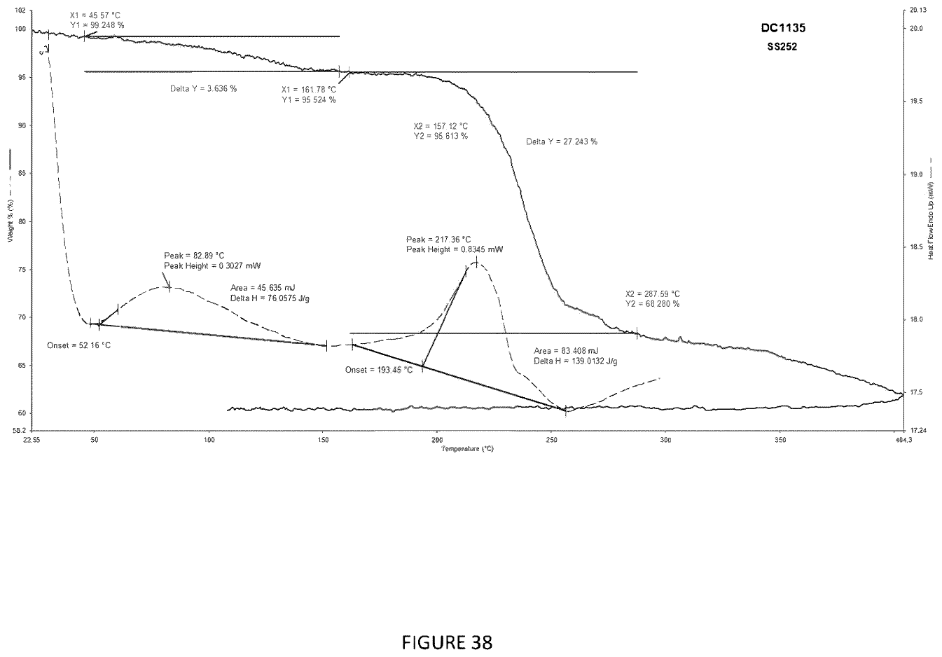

XRPD analysis showed the formation of D-LSD oxalate salt as an amorphous solid. DSC/TGA thermograms showed a first endo onset at 52° C., followed by the melting endo at 193° C. This melting temperature was lower than that of the crystalline version of the oxalate salt identified during salt screening which had a melt temperature of 213° C. A weight loss of 3.6% w/w (0.83 mol eq. of water) was observed from 45-161° C. NMR analysis suggests that this relates to the loss of entrapped ethanol although the level is insufficient for a formal mono solvate. The NMR spectrum of the proton conformed to the molecular structure. FIG. 38 shows DSC and TGA thermographs of D-LSD oxalate amorphous material.

### Compression

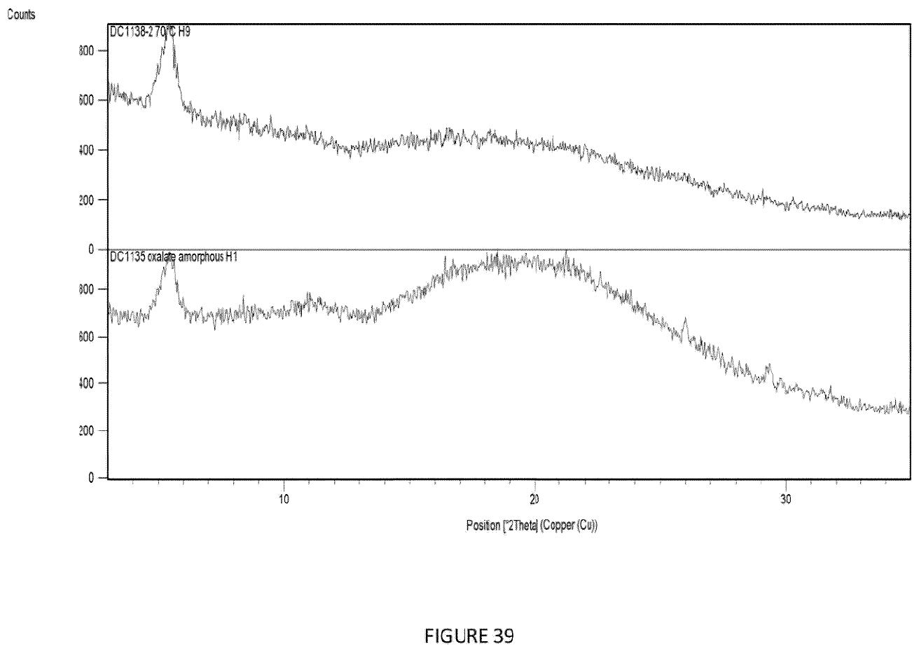

Experimental: D-LSD amorphous oxalate (Lot: DC1134, ~50 mg) was pressed with a force of 105N for approximately 24 hours. The solids were then removed from the press for analysis by XRPD. After the compression experiment, LSD amorphous oxalate salt remained as an amorphous solid. FIG. 39 shows XRPD patterns of solids isolated following compression, D-LSD amorphous oxalate salt reference (bottom) and LSD amorphous oxalate salt isolated after the compression experiment (top).

### Example 32-Heat Cycled Maturation of D-LSD Oxalate Salt (DC1108)

The amorphous oxalate salt was subjected to heat cycled maturation in a range of solvents.

Experimental: Amorphous D-LSD oxalate (lot: DC1135; 24×50 mg) was weighed into crystallization tubes, solvent (various vol charges) was added, and the mixtures equilibrated at 25° C. for ca. 15 minutes. Solvent was then added, and temperature increased to assess for dissolution and to provide a series of maturations/recrystallizations, increasing systematically up to 70° C. and 20 volumes.

The mixtures were thermally cycled between 7° and 25° C. over 48 hours with a minimum of 5 hours at temperature during heating phases. Solids were isolated at two temperatures, at 25° C. during the last cycle and also at elevated temperature.

Lower boiling solvents were equilibrated up to 45° C. Any solutions were clarified to clean, hot tubes to deliver unbiased recrystallizations and isolated either following cooling or via slow evaporation.

All solids were isolated by filtration and liquors were analysed by HPLC to obtain solubility measurements versus a standard of known concentration. Solids were selected for purity assessment to inform of impurity rejection and/or potential stability issues.

All solids were dried at 45° C. in vacuo ahead of analysis.

The form fate of the solids following equilibration is presented in TABLE 2.

TABLE 2

| | | | | Solubility |
|---|---|---|---|---|
| | | Form fate by XRPD | | 25° C. |
| ID | Solvent | 25° C. | 45° C./70° C. | (mg/ml) |
| 3 | TBME | Pattern B | Pattern B | — |
| 5 | Me—THF | amorphous + | amorphous + | 6.14 |
| | | peaks | peaks | |
| 6 | CPME | amorphous + | Insuff. | — |
| | | peaks | material | |
| 7 | 1,4-dioxane | Pattern C | Pattern C | 9.20 |
| 8 | EtOAc | amorphous + | amorphous + | 3.42 |
| | | Pattern B | peaks | |
| | | peaks | | |
| 9 | IP AC | Pattern B | Pattern B | 4.63 |
| 10 | acetone | Pattern B | Pattern F | |
| 11 | MEK | Pattern B | Pattern C | 2.61 |
| 12 | MIBK | Pattern B | Pattern B | 1.60 |
| 13 | Anisole | Pattern F | Pattern B | — |
| 14 | toluene | Amorphous + | Pattern C | — |
| | | Pattern C | | |
| 15 | Chlorobenzene | Pattern A | Pattern A | 2.30 |
| 16 | MeCN | Pattern B | Pattern B | 5.49 |
| 18 | heptane | Pattern B | Pattern B | — |
| 20 | nitromethane | Pattern G | Pattern D | 6.49 |
| 21 | DCM | Pattern E | Pattern E | — |
| 22 | THF: | Pattern B | Insuff. | 8.67 |
| | water (95:5) | | material | |
| 23 | iPrOAc: | Pattern B + | Insuff. | — |
| | water (98:2) | Pattern C | material | |

The API was shown to have excellent solubility of ≥200 mg/ml at 25° C. in the following solvents and solvent mixtures: EtOH and MeOH.

The majority of the solids isolated from the screen at both 25° C. and at elevated temperatures corresponded to a new pattern designated as Pattern B. Pattern A was isolated following maturation in chlorobenzene. Pattern E was isolated once following maturation in DCM. Pattern D material was isolated from maturation in nitromethane as a solvent at 70° C. and the powder converted to Pattern G using the same solvent at 25° C.

A representative sample of Pattern B (Lot: DC1136-18) was analysed by DSC. The thermogram showed endo onset at 176 and 204° C., respectively.

D-LSD oxalate Pattern C (Lot: DC1136-11) was characterized by DSC and TGA. DSC thermogram illustrated a first broad endo onset at 41° C., a second endo onset at 137° C. and a third endo onset at 200°. The TGA showed a weight loss of 0.6% w/w between 25-75° C. which corresponded with the first endotherm in the DSC and a second weight loss of 0.6% w/w between 75-210° C.

D-LSD oxalate Pattern E material was characterized by DSC and TGA. DSC thermogram showed a poor thermal profile with 2 endo onsets at 44 and 207° C. The corresponding TGA illustrated a weight loss of 2.4% w/w between 80-150° C.

D-LSD oxalate Pattern G material was characterized by DSC and TGA. DSC thermogram showed a first endo onset at 119° C. followed by the melting endo at 204° C. The TGA showed a weight loss of 1.38% w/w between 80-160° C.

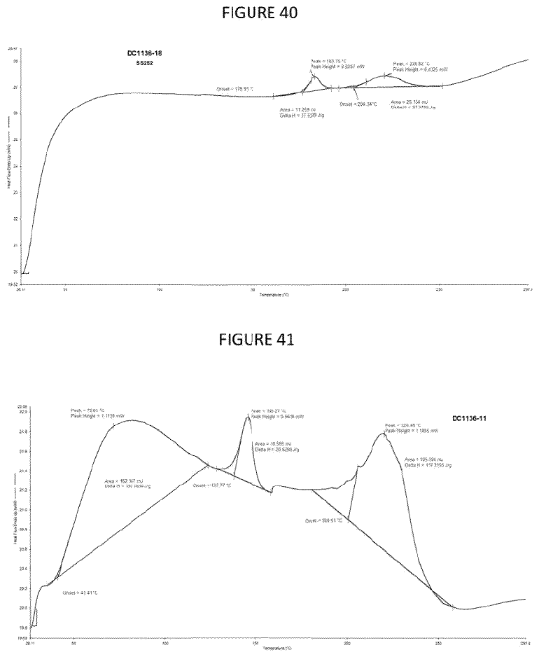

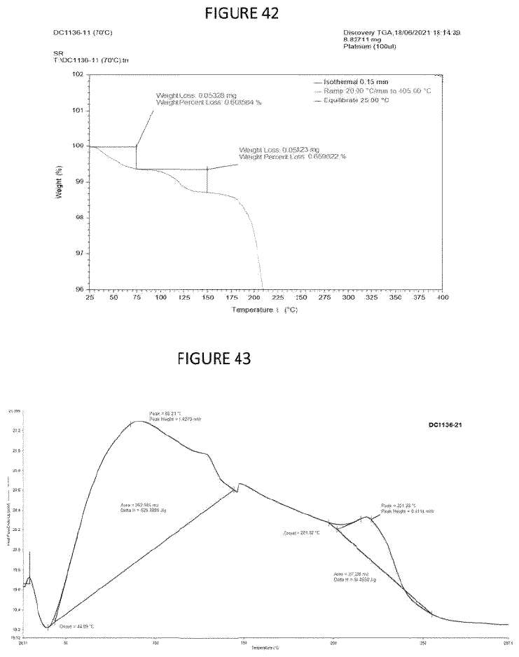

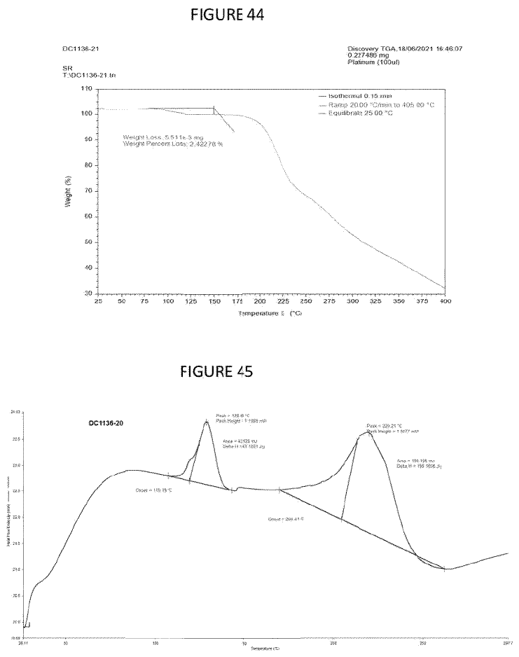

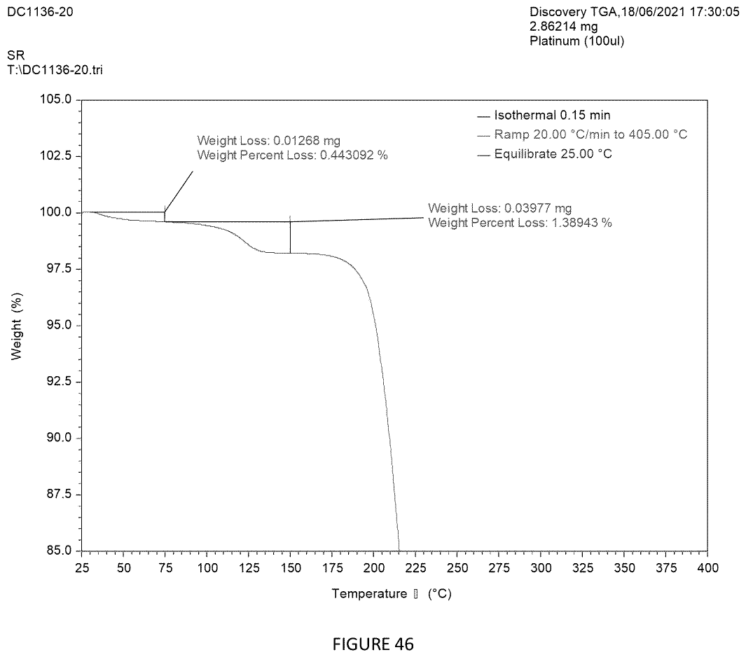

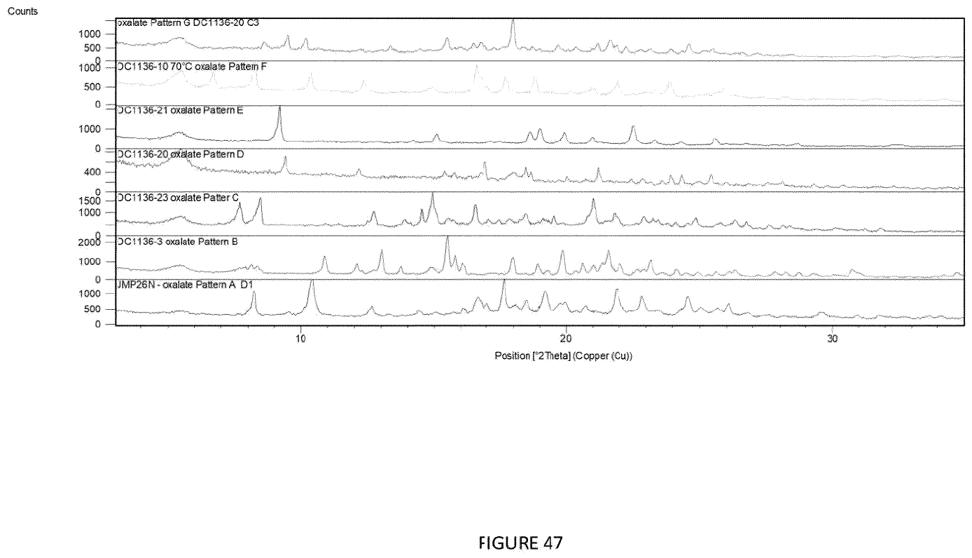

FIG. 40 shows a DSC thermogram of D-LSD oxalate Pattern B. FIG. 41 shows a DSC thermogram of D-LSD oxalate Pattern C. FIG. 42 shows a TGA thermogram of D-LSD oxalate Pattern C. FIG. 43 shows a DSC thermogram of D-LSD oxalate Pattern E. FIG. 44 shows a TGA thermogram of D-LSD oxalate Pattern E. FIG. 45 shows a DSC thermogram of D-LSD oxalate Pattern G. FIG. 46 shows a TGA thermogram of D-LSD oxalate Pattern G. FIG. 47 shows XRPD patterns overlap of the Pattern A (bottom), Pattern B, Pattern C, Pattern D, Pattern E, Pattern F and Pattern G (top) isolated after the maturation experiments.

### Example 33-Amorphous Phosphate Salt

### Preparation of Amorphous Phosphate Salt

Accessing the amorphous phase of a solid is very useful in polymorph screening as it is a metastable, high-energy phase which will often readily reorganise into more thermodynamically stable crystalline forms.

Generation of amorphous D-LSD phosphate salt was attempted using a freeze-drying method from water.

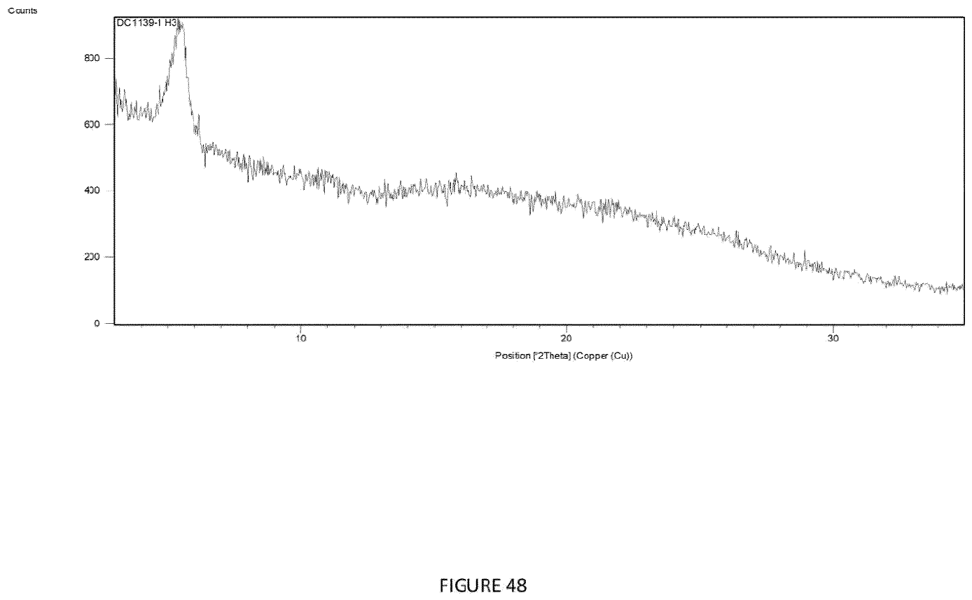

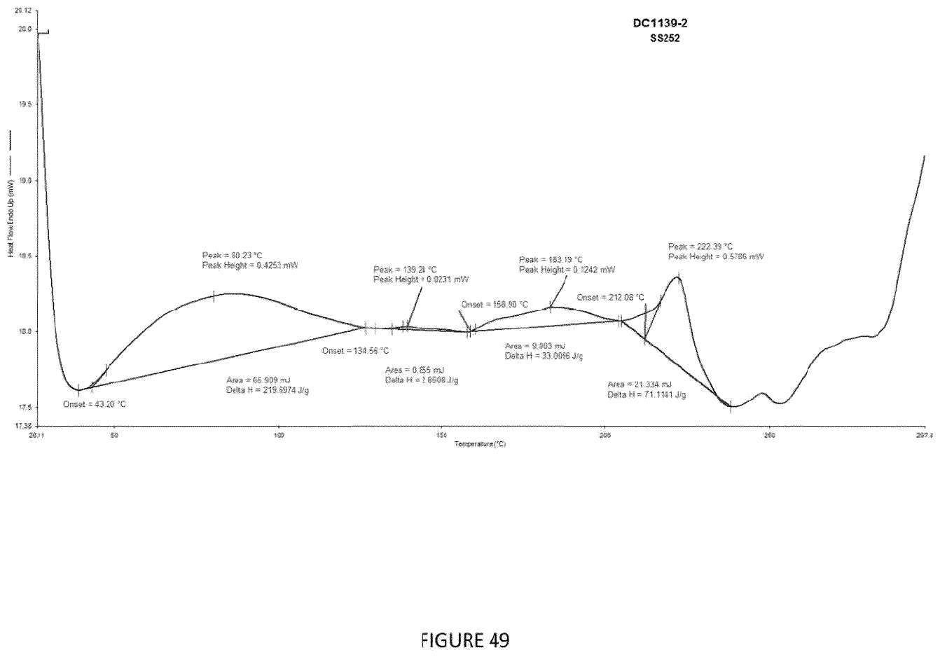

The material recovered was analyzed by XRPD and DSC. The amorphous phase was successfully generated in FIG. 48. Thermal analysis showed a relatively complicated thermal profile (FIG. 49). The profile was typical of an amorphous solid with poorly defined thermal events with four corresponding endotherms observed by DSC at 43, 134, 158 and 212° C.

### Example 34

### Polymorph Screen for D-LSD Phosphate Salt

### Solvent Maturation of Amorphous D-LSD Phosphate Salt (DC1139)

A series of solvent mediated equilibrations with temperature oscillation of the amorphous phase of D-LSD phosphate were conducted.

Experimental: Amorphous D-LSD phosphate (DC1139-2) (20×30 mg) was weighed into crystalliation tubes, solvent (20 vol) was charged, and the mixtures equilibrated at 25° C. for ca. 15 minutes. The mixtures were thermally cycled between 5° and 25° C. over 48 hours. Solids were isolated at 50° C. and 25° C. by filtration and dried in vacuo at 45° C. for 18 hours prior to data collection.

The recovered solids were analysed by XRPD and Pattern C was mainly isolated.

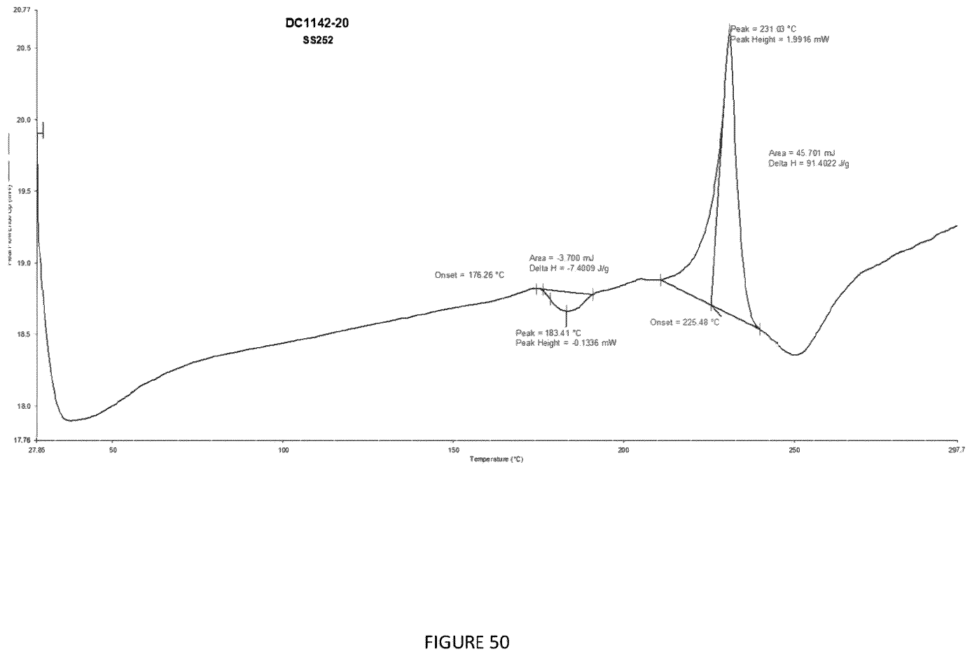

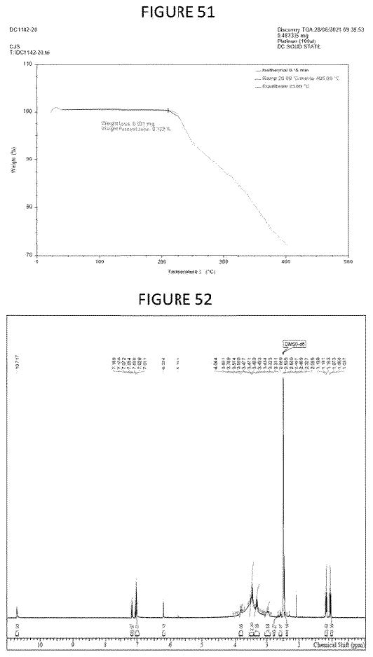

A new pattern, Pattern D, was isolated from nitromethane at both 50° and 25° C. Thermal analysis of Pattern D (FIG. 50) showed an exo onset at ~176° C. followed by the melting onset at 225° C. Analysis by TGA (FIG. 51) showed a minimal weight loss (0.12% w/w) between 40-210° C. ¹H NMR spectrum conformed to the molecular structure and no residual solvent was observed. FIG. 5 shows Pattern A reference (bottom first trace); Pattern B reference (second trace); Pattern C reference (third trace); Pattern D isolated after maturation in nitromethane at 50° C. (fourth trace); Pattern D isolated after maturation in nitromethane at 25° C. (fifth trace).

Pattern D represents an anhydrous phase that likely recrystallizes to Pattern C as shown in the DSC thermogram of the solid. FIG. 52 is a ¹H NMR spectrum of Pattern D.

### Anti-Solvent Mediated Crystallization of D-LSD Phosphate Salt (DC1145)

Anti-solvent mediated crystallization is a useful method for identifying new forms or versions of a solid as once dissolved, the API has no form memory and can recrystallize without bias. Solubility assessment of the API during the initial solvent maturation experiment had identified several solvents and anti-solvents to be used.

Experimental: D-LSD phosphate salt stock solutions were prepared by dissolving the material (200 mg, DC1134) in the minimum amount of water and DMSO. The stock solutions were then clarified into clean sample vials. Aliquots (25 mg API) of the stock solutions were placed into 16 crystallization tubes, pre-heated to 50° C. (30° C. for DCM). Anti-solvents, pre-heated to 50° C. were then added in 2 volume charges until a hazy solution was seen or until 20 volumes of anti-solvent were added. The solutions were then cooled and equilibrated at 25° C. for 24 hours.

### Results and Observations are Presented in TABLE 3.

XRPD analysis showed that Pattern C was mainly isolated, and Pattern B was obtained from water and nitromethane. Pattern B had previously been obtained from a reactive crystallization of the salt in ethanol and was isolated following equilibration of Pattern B in IPA/water. The solutions obtained were placed in a fridge and will be observed after one week.

TABLE 3

| | | | | Observations | Observations | |
|---|---|---|---|---|---|---|
| | | | Volume | at 50° C./ | at ambient | Form |
| ID | Solvent | Anti-solvent | added | 30° C. | temperature | by XRPD |
| DC1145-1 | water | acetone | 20 | Solution | Solution | — |
| DC1145-2 | (10 vol) | MeCN | 20 | Solution | Solution | — |
| DC1145-3 | | 1,4-dioxane | 20 | Solution | Solution | — |
| DC1145-4 | | EtOH | 20 | Solution | Solution | — |
| DC1145-5 | | MeOH | 20 | Solution | Solution | — |
| DC1145-6 | | THF | 20 | Solution | Solution | — |
| DC1145-7 | | DMF | 20 | Solution | Solution | — |
| DC1145-8 | | IPA | 20 | Suspension | Suspension | Pattern B |
| DC1145-9 | DMSO | acetone | 20 | Small | Suspension | Pattern C |
| | (10 vol) | | | transparent | | |
| | | | | crystals | | |
| DC1145-10 | | MTBE | 15 | Solution | Solution | — |
| DC1145-11 | | 1,4-dioxane | 20 | Solution | Solution | — |
| DC1145-12 | | EtOH | 20 | Suspension | Suspension | Pattern C |
| DC1145-13 | | MeOH | 20 | Solution | Solution | — |
| DC1145-14 | | THF | 20 | Solution | Solution | — |
| DC1145-15 | | DMF | 20 | Suspension | Suspension | Pattern C |
| DC1145-16 | | MEK | 20 | Suspension | Suspension | Pattern C |

Pattern B material was analysed by XRPD, DSC and proton NMR.

The XRPD pattern of Pattern B isolated from IPA/water was similar to the Pattern B isolated after the scale up in EtOH. The phases are isostructural solvates. The Pattern B isolated after the scale up was an ethanol solvate, whereas the Pattern B isolated from IPA/water was an IPA solvate.

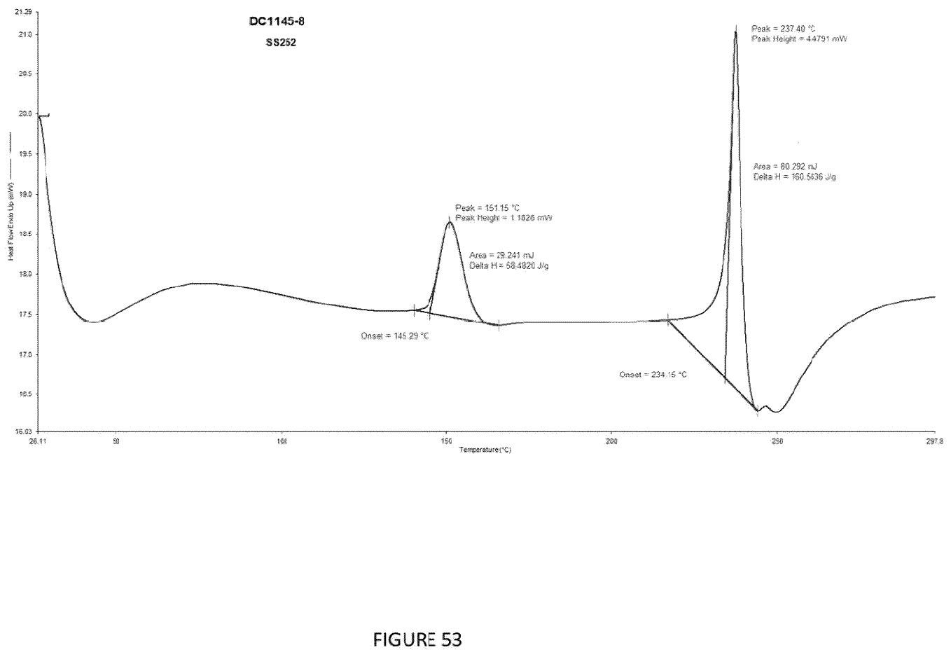

Thermal analysis of Pattern B isolated from water/IPA showed a first endo onset at 145° C., followed by the melting endo onset at 234° C. (FIG. 53). The proton NMR spectrum corresponded to the molecular structure and the presence of IPA was confirmed (~0.5 mol eq.).

### Solvent Vapour Diffusion of Amorphous D-LSD Phosphate (DC1140)

An investigation of the propensity of amorphous D-LSD phosphate to polymorphism was conducted using the solvent vapour diffusion technique with amorphous input. This technique uses a less forcing system and allows an API to order itself from surface interaction of solvent to induce crystallization.

Experimental: Solvent vapour chambers were prepared by dosing solvent (3 ml, TABLE 4) into a glass vial. Into this vapour chamber was placed a smaller glass vial containing amorphous API (30 mg, DC1134). The chamber was sealed and allowed to equilibrate without disturbance for 2 weeks ahead of observation and XRPD analysis. Results and observations are shown in TABLE 4.

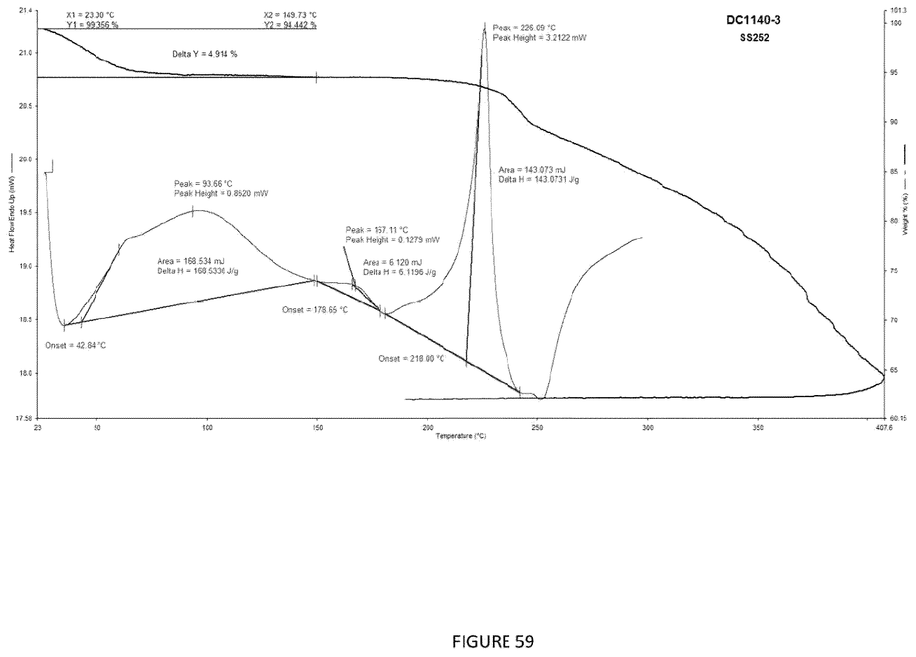

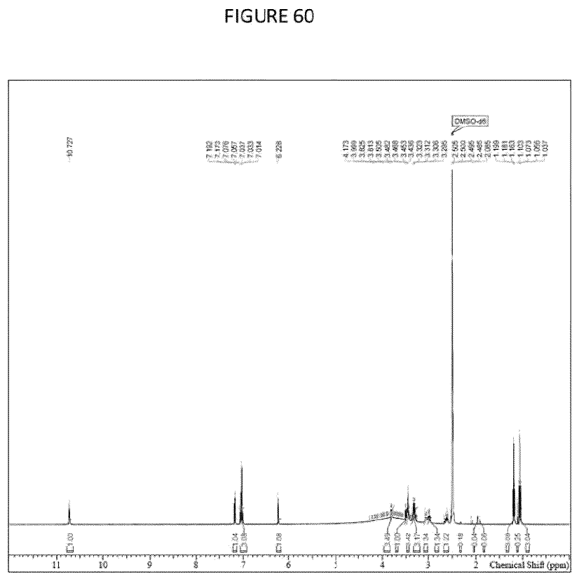

D-LSD phosphate Pattern C salt was mainly recovered, but a new pattern named Pattern E was isolated. This material was characterized by XRPD (FIG. 58), DSC, TGA and proton NMR. DSC thermogram (FIG. 59) showed a first endo onset at 42° C., a second endo onset at 178° C., followed of the endo melting onset at 218° C. TGA analysis showed a weight loss of 4.9% (0.25 mol eq. of MTBE) between 29-149° C. ¹H NMR spectrum conformed to the molecular structure and the presence of MTBE was observed (~0.1 mol eq.). FIG. 60 is a ¹H NMR spectrum of D-LSD phosphate Pattern E.

TABLE 4

| | | Observation | XRPD | XRPD |
|---|---|---|---|---|
| Sample ID | Solvent | at 1 week | at 1 week | at 2 weeks |
| DC1140-1 | EtOH | beige solid | beige solid | Pattern B |
| DC1140-2 | MeOH | brown solid | brown solid | Pattern C |
| DC1140-3 | TBME | beige solid | beige solid | Pattern E |
| DC1140-4 | THF | beige solid | beige solid | Pattern C |
| DC1140-5 | 1,4-dioxane | beige solid | beige solid | Pattern C |
| DC1140-6 | EtOAc | beige solid | beige solid | Pattern C |
| DC1140-7 | acetone | brown solid | brown solid | Pattern C |
| DC1140-8 | Cl-benzene | beige solid | beige solid | peaks |
| DC1140-9 | MeCN | brown solid | brown solid | Pattern C |
| DC1140-10 | heptane | beige solid | beige solid | amorphous + |
| | | | | peaks |
| DC1140-11 | nitromethane | beige solid | beige solid | Pattern C |
| DC1140-12 | DCM | brown solid | brown solid | Pattern D |

### Mixed Solvent Cooling Crystallization of D-LSD Phosphate Salt (DC1149)

Investigations identified a range of solvents and anti-solvents of the API which can be exploited to provide a variety of modes of crystallization. The form fate of D-LSD oxalate salt was further assessed by a series of mixed solvent cooling crystallizations.

Experimental: Amorphous D-LSD phosphate salt (DC1135, 8×25 mg) was weighed into crystallization tubes and suspended in the relevant anti-solvent (detailed in TABLE 5) (10 vol). Temperature was increased to 50° C. and solvent was added in aliquots until a solution was achieved. Solutions were cooled and equilibrated at room temperature for 16 hours. Solids were isolated by filtration and dried in vacuo at 40° C. for 18 hours prior to data collection.

Results and observations shown in TABLE 5.

The suspension recovered were filtered and analysed by XRPD. Pattern B was isolated from EtOH/water. Pattern C was isolated from MeOH/water, 1,4-dioxane/water and MeCN/water. The solutions obtained were slow evaporated and gel samples were recovered.

TABLE 5

| | | | Volume of | | | |
|---|---|---|---|---|---|---|
| | | | solvent | Obs | Obs | |
| Entry | Anti-Solvent | Solvent | added | 50° C. | RT cool | XRPD |
| DC1149-1 | EtOH | water | 4 | Suspension | Suspension | Pattern B |
| DC1149-2 | MeOH | | 2 | Suspension | Suspension | Pattern C |
| DC1149-3 | THF | | 2 | Solution | Solution | gel |
| DC1149-4 | Acetone | | 4 | Suspension | Suspension | Amorphous + |
| | | | | | | peaks |
| DC1149-5 | 1,4-dioxane | | 4 | Suspension | Suspension | Pattern C |
| DC1149-6 | IPA | | 10 | Solution | Solution | gel |
| DC1149-7 | MeCN | | 4 | Solution | Solution | Pattern C |
| DC1149-8 | butanol | | 4 | Solution | Solution | gel |

### Example 35 Polymorph Screen for D-LSD Oxalate Salt

### Mixed Solvent Cooling Crystallizations of D-LSD Oxalate Salt (DC1143)

Investigations conducted during the programme of works identified a range of solvents and anti-solvents of the API which can be exploited to provide a variety of modes of crystallization. The form fate of D-LSD oxalate salt was further assessed by a series of mixed solvent cooling crystallizations.

Experimental: Amorphous D-LSD oxalate salt (DC1135, 8×25 mg) was weighed into crystallization tubes and suspended in the relevant anti-solvent (detailed in TABLE 6) (10 vol). Temperature was increased to 50° C. and solvent was added in aliquots until a solution was achieved. Solutions were cooled and equilibrated at room temperature for 16 hours. Solids were isolated by filtration and dried in vacuo at 40° C. for 18 hours prior to data collection.

Results and observations shown in TABLE 6.

TABLE 6

| | | | Volume of | | | | |
|---|---|---|---|---|---|---|---|
| | | | solvent | Obs | Obs | Slow | |
| Entry | Anti-Solvent | Solvent | added | 50° C. | RT cool | evaporation | XRPD |
| DC1143-9 | heptane | MeOH | 36 | Solution- | Suspension | — | Pattern B |
| | | | | precipitated | | | |
| | | | | quickly | | | |
| DC1143-10 | TBME | | 36 | Solution- | Suspension | — | Pattern B |
| | | | | precipitated | | | |
| | | | | quickly | | | |
| DC1143-11 | THF | | 15 | Solution | Solution | Gel | — |
| DC1143-12 | toluene | | 15 | Solution | Solution | Solid | Pattern B |
| DC1143-13 | MeCN | | 9 | Solution | Suspension | — | Pattern B |
| DC1143-14 | Ethyl acetate | | 10 | Solution | Solution | Solid | Pattern B |
| DC1143-15 | 1,4-dioxane | | 10 | Solution | Solution | Gel | — |
| DC1143-16 | acetone | | 15 | Solution | Suspension | — | Pattern B |

### Example 36

### Polymorph Screen for D-LSD Sulfate Salt

### Slurry Experiments (DC1162)

Sufficient D-LSD sulfate salt (lot: DC1151) was added to a given solvent until undissolved solids remained at the desired temperature (25° C.). The crystallization tube was sealed, and the slurry was maintained at the selected temperature and agitated by magnetic stirring for 3 days. Solids were isolated by filtration through a 0.45 μm PTFE filter prior to analysis by XRPD. The results are shown in TABLE 7. Gel samples were mainly obtained.

TABLE 7

| Sample | Solvent | Result | XRPD |
|---|---|---|---|
| DC1162-1 | cyclohexane | gel | — |
| DC1162-2 | heptane | gel | — |
| DC1162-3 | MIBK | gel | — |
| DC1162-4 | TBME | gel | — |
| DC1162-5 | THF | gel | — |
| DC1162-6 | acetone | gel | — |
| DC1162-7 | DCM | gel | — |
| DC1162-8 | EtOH | solid | amorphous |
| DC1165-1 | EtOH/water 98:2 | gel | — |

### Example 37

### Polymorph Screen for D-LSD HBr Salt

### Slurry Experiments

Sufficient D-LSD HBr salt (lot: DC1152) was added to a given solvent until undissolved solids remained at the desired temperature (25° C.). The crystallization tube was sealed, and the slurry was maintained at the selected temperature and agitated by magnetic stirring for 3 days. Solids were isolated by filtration through a 0.45 μm PTFE filter prior to analysis by XRPD. The results are shown in TABLE 8. A new pattern named D-LSD HBr Pattern C was isolated and was characterized by DSC, TGA and ¹H NMR.

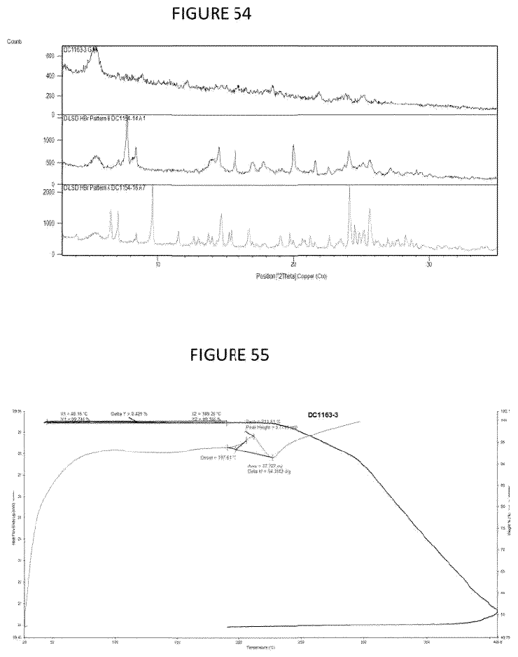

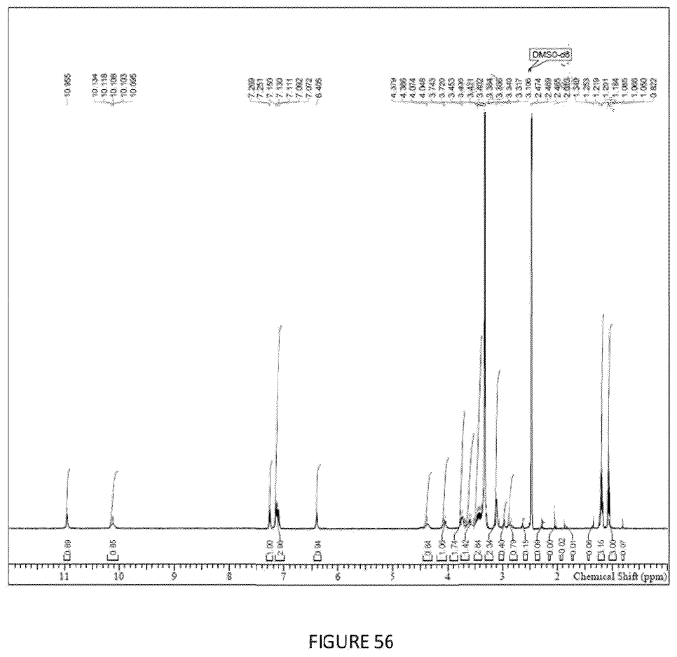

The XRPD results showed that the material exhibited low crystallinity (FIG. 54). The DSC and TGA analyses illustrated a weight loss of 0.42% (w/w) between 46-189° C. and a melting onset at ~197° C. (FIG. 55). The ¹H NMR spectrum conformed to the molecular structure and traces of impurities were detected (FIG. 56).

TABLE 8

| Sample | Solvent | Result | XRPD |
|---|---|---|---|
| DC1163-1 | MeCN | solid | Pattern A |
| DC1163-2 | heptane | solid | amorphous + |
| | | | Pattern A |
| DC1163-3 | MIBK | solid | Pattern C |
| DC1163-4 | TBME | solid | amorphous |
| DC1163-5 | IPAC | solid | Pattern A |
| DC1163-6 | EtOH | solid | amorphous |
| DC1163-6 | EtOAc: water (98:2 v/v) | solid | Pattern A |

### Water Mixture Solvent Cooling Crystallization (DC1164)

The form fate of D-LSD HBr salt (lot: DC1152) was further assessed by a series of water mixed solvent cooling crystallizations.

Experimental: D-LSD HBr salt (lot: DC1152, 6×~25 mg) was charged to crystallization tubes and suspended in 5-10 vols of the appropriate anti-solvent. The mixtures were heated to 50° C. and stirred and equilibrated at 50° C. for ca. 30 minutes and then cooled to 25° C. and equilibrated for ca. 20 hours. The mixtures that returned a suspension at RT were isolated in vacuo and the solids dried at 45° C. for 20 hours ahead of characterization.

Results are summarized in TABLE 9. D-LSD HBr Pattern A was mainly recovered.

TABLE 9

| | | Volume | | |
|---|---|---|---|---|
| Sample | Solvent | added | Result | XRPD |
| DC1164-2 | EtOAc: water 95:5 | 10 | solid | Pattern A |
| DC1164-3 | EtOAc: water 90:10 | 5 | gel | — |
| DC1164-4 | EtOAc: water 85:15 | 5 | solid | Pattern A |
| DC1164-6 | ACN: water 95:5 | 5 | solid | Pattern A |
| DC1164-7 | ACN: water 90:10 | 5 | solid | Pattern A |
| DC1164-8 | ACN: water 85:15 | 5 | solid | Pattern A |

### Crash Precipitation of D-LSD HBr Salt (DC1167)

The crash precipitation of a solution of API into various anti-solvents is a way to assess any potential metastable forms of a crystalline solid.

Experimental: D-LSD HBr salt (DC1152) was weighed into a COC vial and dissolved in the appropriate solvent (EtOH, 10 vol at 50° C.). The solutions were then clarified into clean tubes in order to have a clear solution, free of any visible particulates. The solutions were then split into 8 aliquots per solvent in order to have ca. 25 mg of material in each aliquot. The aliquots were then transferred as one single charge into crystallization tubes charged of 10 vols of the appropriate anti-solvent and pre-cooled to 0° C. The mixtures were equilibrated for a maximum of 30 minutes at 0° C. Those entries that afforded a suspension were isolated and the solids dried at 45° C. for ca. 20 hours ahead of characterization. Amorphous materials were mainly recovered, and Pattern A was isolated from EtOH/heptane (TABLE 10).

TABLE 10

| | | | Suspension | |
|---|---|---|---|---|
| Sample | Solvent | Antisolvent | achieved (✓/X) | XRPD |
| DC1167-1 | EtOH | toluene | ✓ | amorphous |
| DC1167-2 | | heptane | ✓ | Pattern A |
| DC1167-3 | | pentane | ✓ | amorphous |
| DC1167-4 | | MIBK | ✓ | amorphous |
| DC1167-5 | | MEK | ✓ | amorphous |
| DC1167-6 | | cyclohexane | ✓ | amorphous |
| DC1167-7 | | TBME | ✓ | amorphous |
| DC1167-8 | | water | X (gel) | — |

### Example 38

### Preparation and Characterization of Amorphous D-LSD Sulfate Salt

D-LSD Free Base (Pattern A, 5.01 g) was placed in a round bottom flask (V=200 mL) and dissolved in EtOH (10 V). The solution was stirred at 300 rpm, at 25° C. for 30 min. Then, a stock solution of sulphuric acid in EtOH (1 M, 15.49 mL) was slowly added. The reaction was carried out at 25° C. overnight. A solution was observed and was reduced in vacuo to deliver a solid. The material recovered was analysed by XRPD, DSC, TGA and ¹H NMR.

Yield=97.5% (6.35 g)

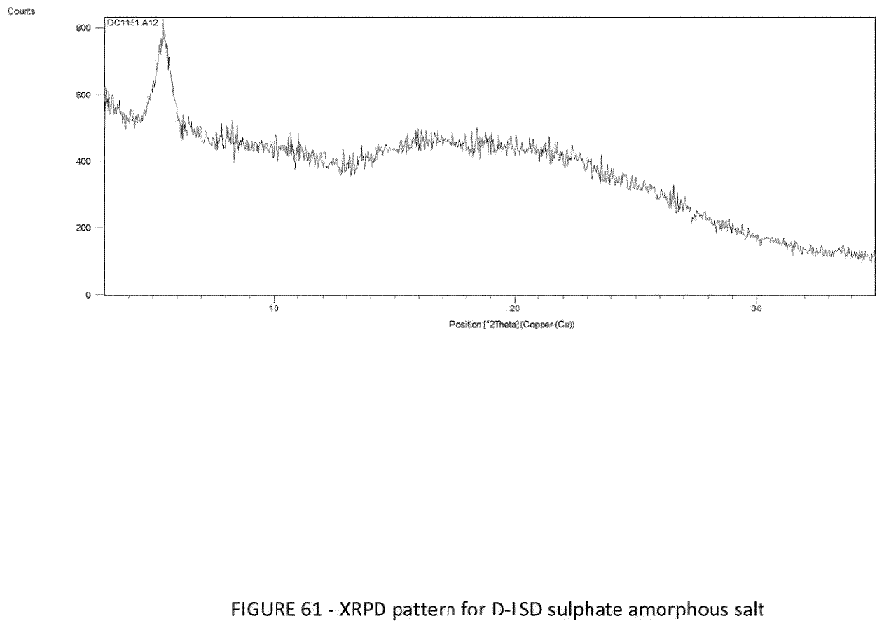

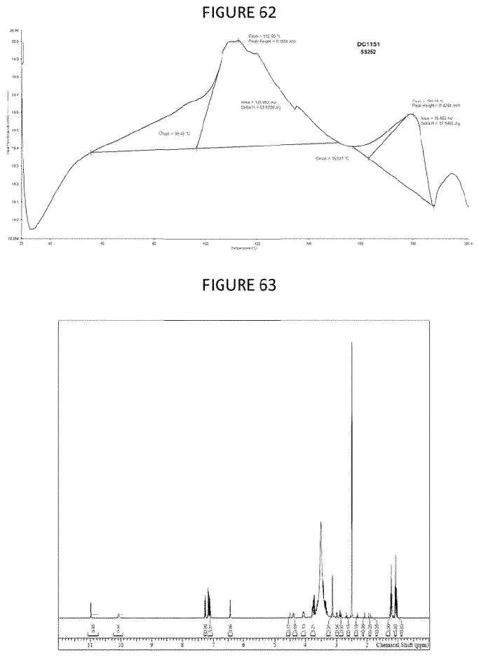

XRPD analysis showed the formation of D-LSD sulfate amorphous salt (FIG. 61). FIG. 62 is a DSC thermogram for D-LSD sulfate amorphous salt.

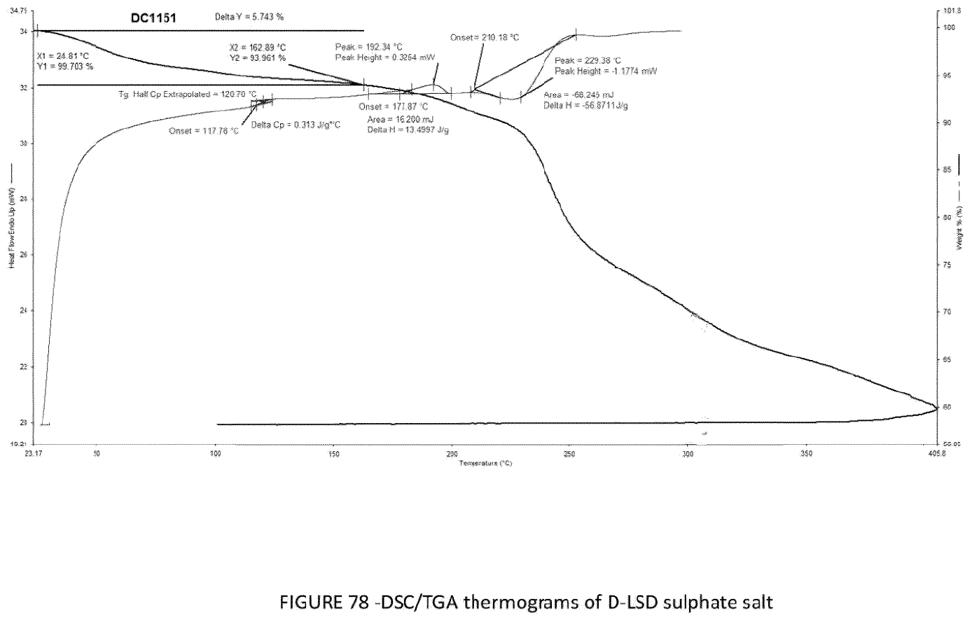

The amorphous D-LSD sulfate salt (lot: DC1151) was characterized by DSC and TGA. DSC analysis showed a possible glass transition at 120° C. (Half Cp) followed by an endo onset at 177° C. and an exo onset at 210° C. TGA analysis showed a weight loss of 5.7% (w/w) between 24-163° C. FIG. 78 shows DSC/TGA thermograms of D-LSD sulfate salt.

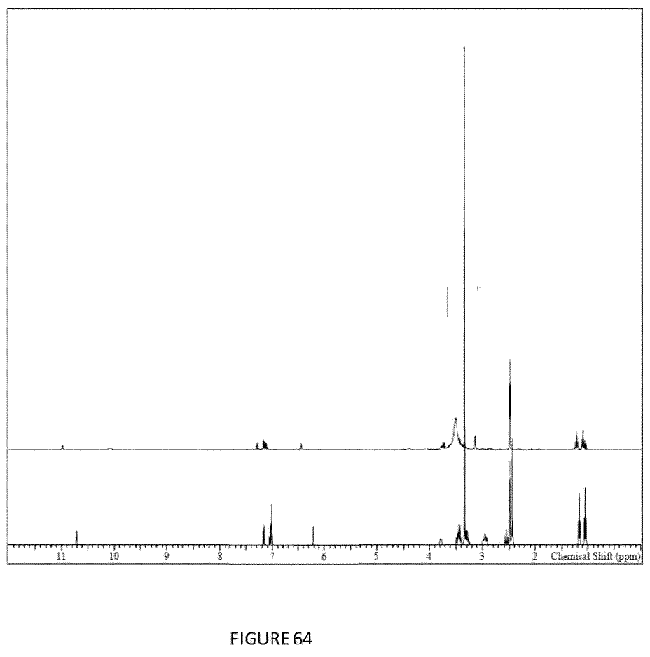

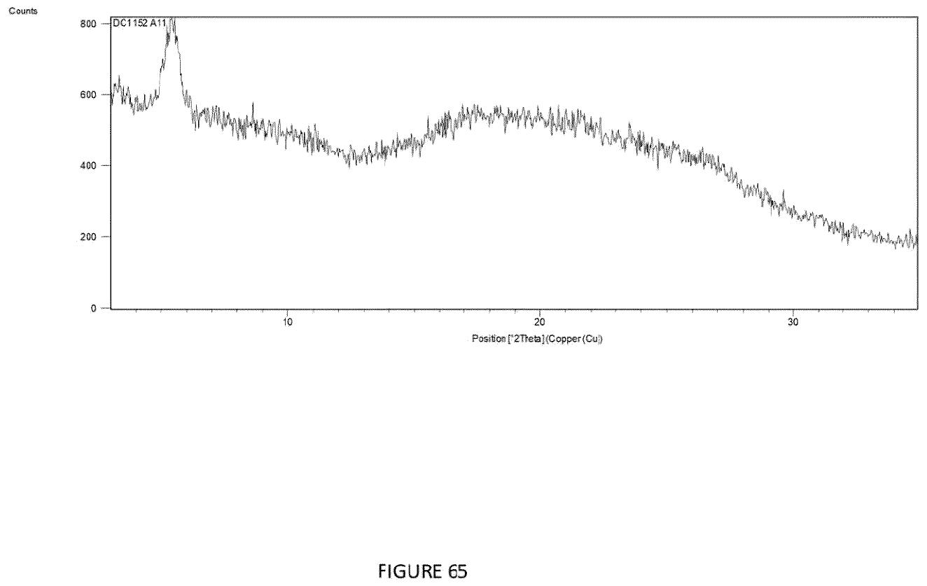

¹H NMR spectrum of D-LSD sulfate salt conformed to the molecular structure and indicated traces of impurities along with solvent content fitting for the DSC profile collected (FIG. 63). ¹H NMR spectrum overlap between D-LSD Free Base and D-LSD sulfate salt showed the anticipated shifts illustrative of salt formation (FIG. 64).

### Example 39

### Preparation of D-LSD Hydrobromic Amorphous Salt

D-LSD Free Base (Pattern A, 2.53 g) was placed in a round bottom flask (V=100 mL) and dissolved in EtOH (10 V). The solution was stirred at 300 rpm, at 25° C. for 30 minutes and a stock solution of HBr (48%) in EtOH (1 M, 7.8 mL) was slowly added. The reaction was carried out at 25° C. overnight. A solution was observed and was reduced in vacuo to deliver a solid. The material recovered was analysed by XRPD, DSC and ¹H NMR.

Yield=93% (2.95 g)

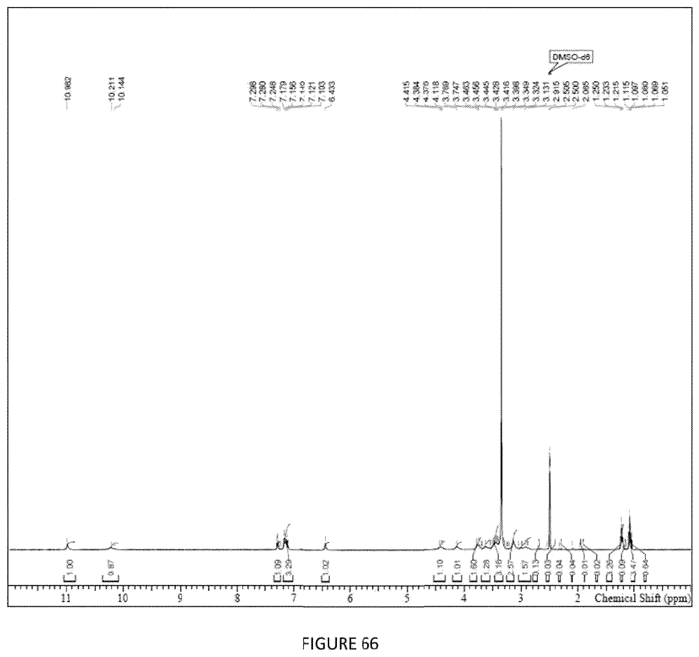

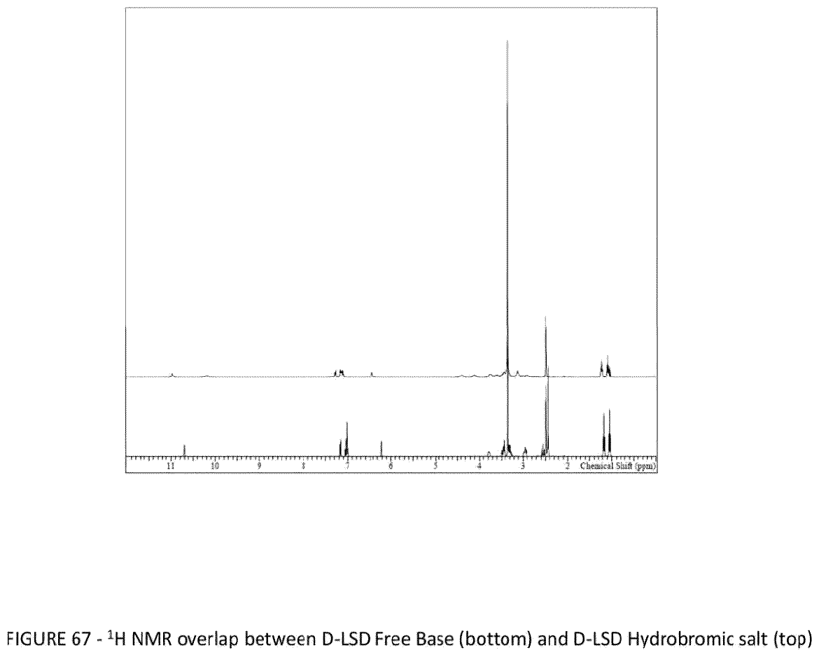

XRPD analysis showed the formation of D-LSD hydrobromic amorphous salt (FIG. 65). ¹H NMR spectrum of D-LSD HBr salt conformed to the molecular structure with trace levels of solvent present (approximately 2 wt % EtOH, FIG. 66). ¹H NMR spectrum overlap between D-LSD (lot; ASH657) and D-LSD HBr salt illustrated the anticipated shifts induced by salt formation with a strong acid (FIG. 67).

### Example 40

Hierarchical investigation between D-LSD phosphate Pattern A, C and D (DC1150)

To further elucidate the relationship between the three anhydrous forms of the API; Patterns A, Pattern C and Pattern D, and to identify the thermodynamically preferred version of D-LSD phosphate salt, hierarchical studies were conducted. Equal amounts of Patterns A, Pattern C and Pattern D were competitively equilibrated in three different solvents and a solvent mixture at 25° C. After 7 days, the solids were isolated and examined by XRPD to assess their form.

Experimental: D-LSD phosphate Pattern A (lot: DC1137-19), D-LSD phosphate Pattern C (lot: DC1137-23) and D-LSD phosphate Pattern D (lot: DC1140-12) were weighed (10 mg each, 1:1:1) into crystallization tubes, solvent (detailed in TABLE 11) charged, samples agitated (25° C., 7 days). Samples dried in vacuo (40° C., 20 hours) ahead of data analysis.

Results shown in TABLE 11.

A complete conversion to Pattern C was observed in THF: water (95:5 v/v), 1,4-dioxane and THF. Instead, a mixture of Pattern A and Pattern C was observed in EtOH.

TABLE 11

| | | Form fate |
|---|---|---|
| ID | Solvent | by XRPD |
| DC1150-1 | THF: water (95:5 v/v) | Pattern C |
| DC1150-2 | 1,4-dioxane | Pattern C |
| DC1150-3 | THF | Pattern C |
| DC1150-4 | EtOH | Pattern A + |
| | | Pattern C |

### Example 41

### Accelerated Storage of D-LSD Phosphate Pattern C and D-LSD Oxalate Pattern B

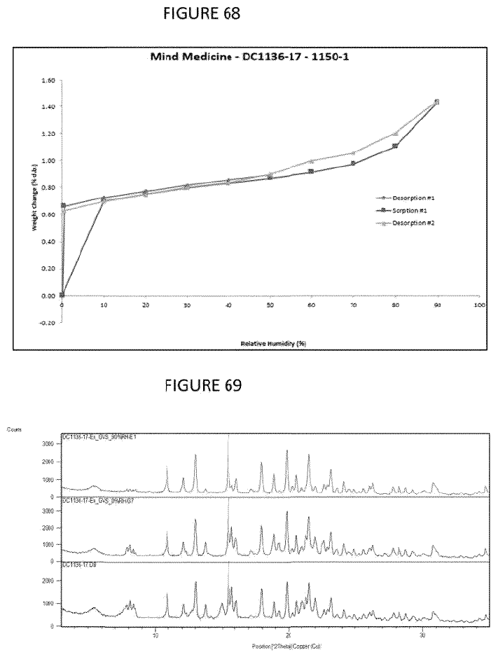

D-LSD phosphate Pattern C (lot: DC1137-9) and D-LSD oxalate Pattern B (lot: DC1144-4) were placed in unsealed vials in a cabinet under controlled conditions at 40° C./75% RH for 14 days. Samples were visually observed for signs of deliquescence and analysed by XRPD post stressing for physical stability and HPLC for chemical stability. FIG. 68 is a DVS profile of D-LSD oxalate Pattern B. FIG. 69 is XRPD patterns of D-LSD oxalate Pattern B ex. DVS post equilibration at 0% RH (middle trace) and 90% RH (top trace) compared to Pattern B reference (bottom trace).

TABLE 12 summarizes the XRPD and HPLC data collected for each solid.

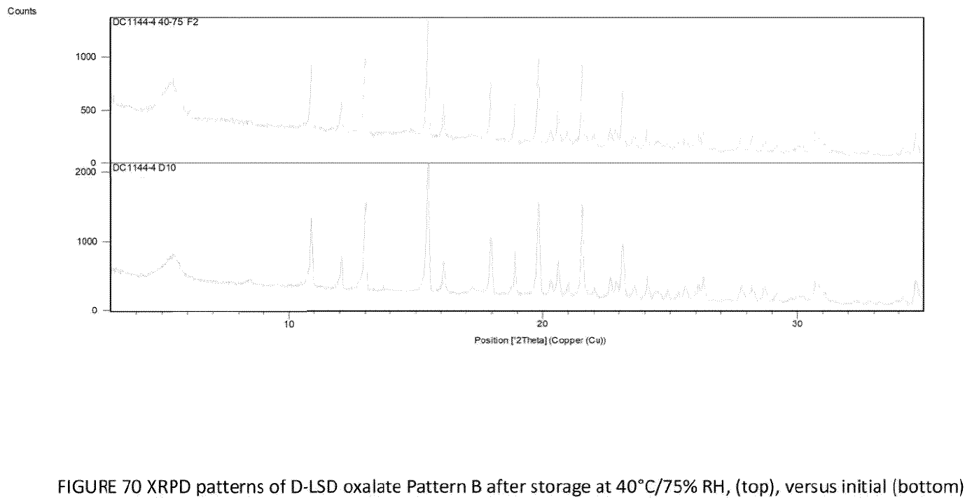

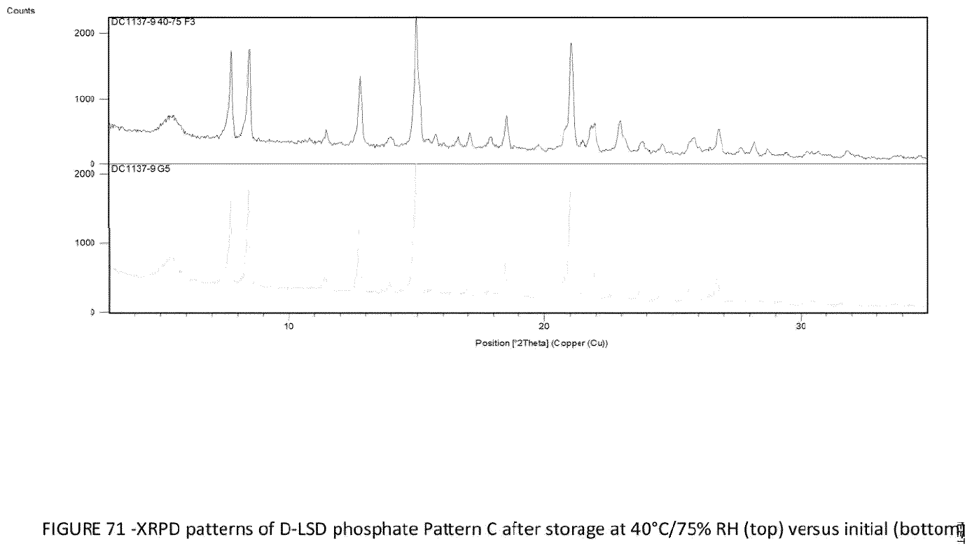

D-LSD oxalate Pattern B and D-LSD phosphate Pattern C after stress at 40° C./75 RH did not show a change in form (FIG. 70 and FIG. 71).

TABLE 12

| | | Nature | |
|---|---|---|---|
| Sample No. | XRPD of Input | of solid | XRPD of Output |
| DC1144-4 40-75 | oxalate Pattern B | solid | oxalate Pattern B |
| DC1137-9 40-75 | phosphate Pattern C | solid | phosphate Pattern C |

### Example 42

### Heat Cycled Maturation of D-LSD Sulfate Amorphous Salt and Initial Solubility Assessment (DC1153)

Experimental: D-LSD sulfate salt (lot: DC151; 24×25 mg) was weighed into crystallization tubes, solvent (various vol charges) was added, and the mixtures equilibrated at 25° C. for ca. 15 minutes. Solvent was then added, and temperature increased to assess for dissolution and to provide a series of maturations/recrystallizations, increasing systematically up to 70° C. and 30 volumes.

The mixtures were thermally cycled between 7° and 25° C. over 48 hours with a minimum of 5 hours at temperature during heating phases. Solids were isolated at two temperatures, at 25° C. during the last cycle and also at elevated temperature.

Lower boiling solvents were equilibrated up to 45° C.

Any solutions were clarified to clean, hot tubes to deliver unbiased recrystallizations and isolated either following cooling or via slow evaporation.

Most of the samples observed at room temperature were gels.

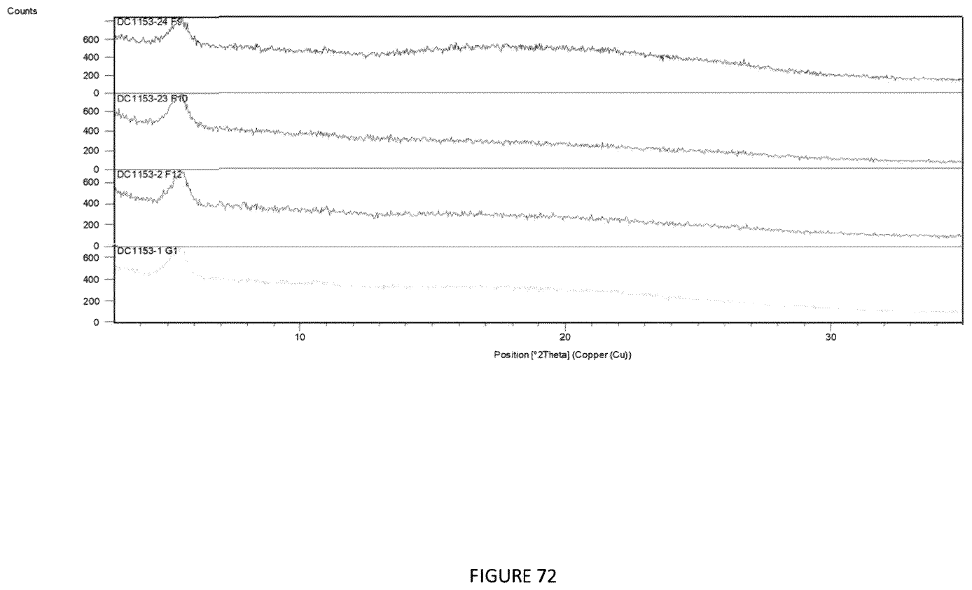

The solids recovered from the solutions by slow evaporation were analysed by XRPD and amorphous material were observed (FIG. 72).

The API had a solubility of ≥200 mg/mL at 25° C. in MeOH, a solubility of >100 mg/mL at 25° C. in EtOH, and a solubility of >100 mg/mL at 40° C. in iPrOAc/water (98:2) and IPA/water (95:5).

### Example 43

### Thermal Characterization of Amorphous D-LSD HBr Salt

DSC thermogram of amorphous D-LSD HBr salt (lot: DC1152) showed an exo onset at 220° C.

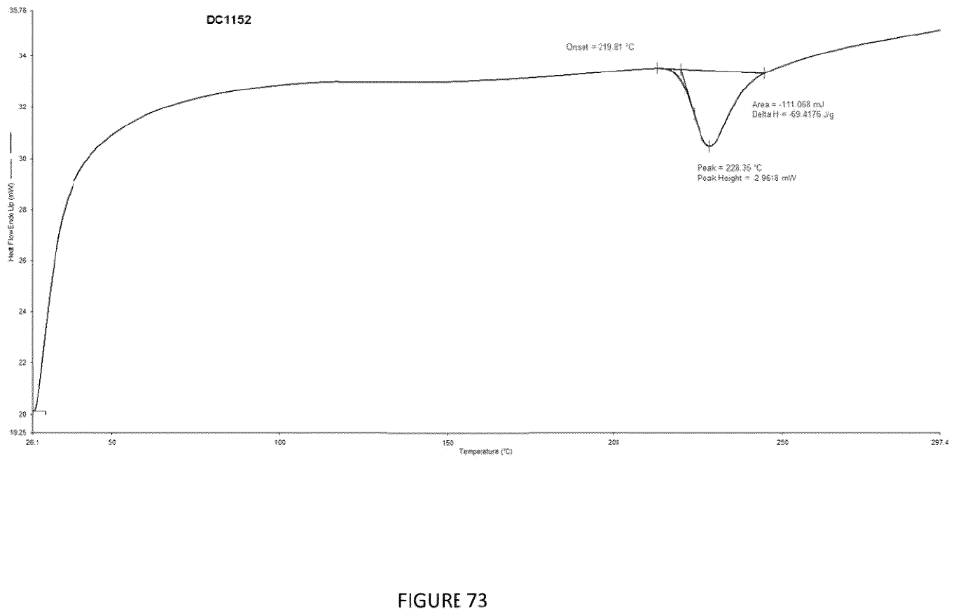

A thermal cycling experiment for the amorphous D-LSD HBr salt was carried out. A small sample (approximately 5 mg) of this material was placed in a TGA pan and heated at 40° C./minute to 190° C., then the sample was cooled to −20° C. The cycle was repeated twice. The thermogram did not reveal any exo or endo event. FIG. 73 shows a DSC thermogram of D-LSD HBr salt.

### Example 44

### Heat Cycled Maturation of D-LSD HBr Amorphous Salt and Initial Solubility Assessment (DC1154)

Heat cycled maturation is a pragmatic method for the assessment of material behaviour across a wide range of solvent chemotypes and mixed solvents. Such experimental modulation illustrates solvent vs anti-solvent and temperature-solubility effects and also informs of potential form changes induced from the input based on solvent type. Whilst a significant seed bed may be maintained from the input form, any solutions obtained via clarification provide a non-biased recrystallization with the potential to deliver new phases for assessment.

Experimental: D-LSD HBr salt (lot: DC1152; 24×25 mg) was weighed into crystallization tubes, solvent (various vol charges) was added, and the mixtures equilibrated at 25° C. for ca. 15 minutes. Solvent was then added, and temperature increased to assess for dissolution and to provide a series of maturations/recrystallizations, increasing systematically up to 70° C. and 30 volumes.

The mixtures were thermally cycled between 7° and 25° C. over 48 hours with a minimum of 5 hours at temperature during heating phases. Solids were isolated at two temperatures, at 25° C. during the last cycle and also at elevated temperature.

Lower boiling solvents were equilibrated up to 45° C.

Any solutions were clarified to clean, hot tubes to deliver unbiased recrystallizations and isolated either following cooling or via slow evaporation.

All solids were isolated by filtration and liquors were analysed by HPLC to obtain solubility measurements versus a standard of known concentration. Solids were selected for purity assessment to inform of impurity rejection and/or potential stability issues.

All solids were dried at 45° C. in vacuo ahead of analysis.

The API had a solubility of ≥200 mg/ml at 25° C. in: MeOH, THF/water (95:5 v/v).

A solubility of >100 mg/mL at 25° C. in: EtOH, MEK, nitromethane.

A solubility of >100 mg/mL at 40° C. in: 1,4-dioxane, iPrOAc/water (98:2 v/v), IPA/water (95:5 v/v).

TABLE 13 shows Form fate, and solubility summary of D-LSD amorphous HBr following equilibration in various solvents with thermal modulation.

TABLE 13

| | | Form fate by XRPD | | | Solubility |
|---|---|---|---|---|---|
| | | | | Slow | 25° C. |
| ID | Solvent | 25° C. | 45° C./70° C. | evaporation | (mg/ml) |
| 1 | EtOH | — | — | amorphous | — |
| 2 | MeOH* | — | — | amorphous | — |
| 3 | TBME* | amorphous | Insuff. | — | 9.84 |
| | | | material | | |
| 4 | THF | — | — | amorphous + | — |
| | | | | peaks | |
| 5 | water | amorphous + | amorphous + | — | 25.1 |
| | | peaks | peaks | | |
| 6 | 2-Me—THF | — | — | amorphous + | — |
| | | | | peaks | |
| 7 | 1,4-dioxane | — | — | amorphous | — |
| 8 | EtOAc | Pattern A | Pattern A | — | 2.63 |
| 9 | IPAC* | amorphous | amorphous | | 4.19 |
| 10 | acetone* | — | — | amorphous | — |
| 11 | MEK | — | — | amorphous | — |
| 12 | MIBK | — | — | amorphous | 16.4 |
| 13 | Anisole | — | — | amorphous | — |
| 14 | toluene | Pattern B | Pattern B | — | 3.91 |
| 15 | chlorobenzene | amorphous | Insuff. | — | 3.12 |
| | | | material | | |
| 16 | MeCN | Pattern A | Pattern A | — | 8.68 |
| 17 | propionitrile | — | — | amorphous | — |
| 18 | heptane | amorphous + | amorphous | — | 0.05 |
| | | peaks | | | |
| 19 | cyclohexane | amorphous + | amorphous + | — | — |
| | | peaks | peaks | | |
| 20 | nitromethane | — | — | amorphous | — |
| 21 | DCM* | — | — | amorphous | — |
| 22 | THF: | — | — | amorphous | — |
| | water (95:5) | | | | |
| 23 | iPrOAc: | — | — | amorphous | — |
| | water (98:2) | | | | |
| 24 | IPA: | — | — | amorphous | — |
| | water (95:5) | | | | |

The majority of the solids isolated from the screen were amorphous materials. A new pattern designated Pattern A was isolated from EtOAc and MeCN. Pattern B was isolated once following maturation in toluene.

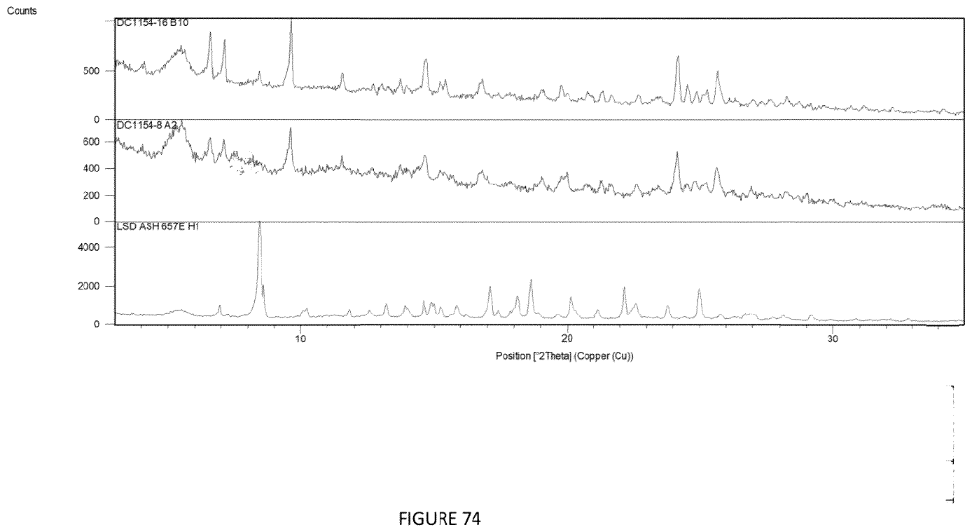

A sample of D-LSD HBr Pattern A was characterized by XRPD (FIG. 74, top).

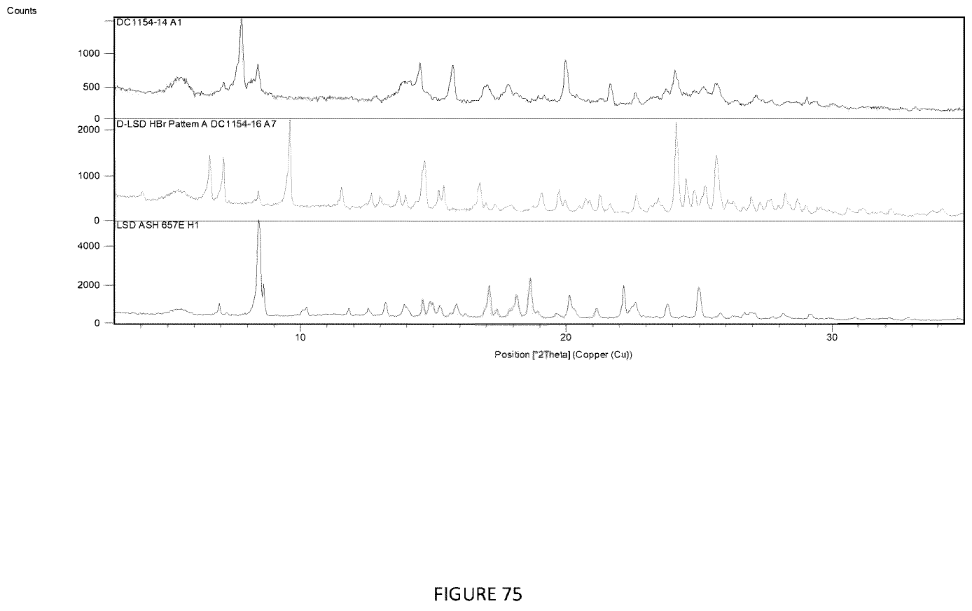

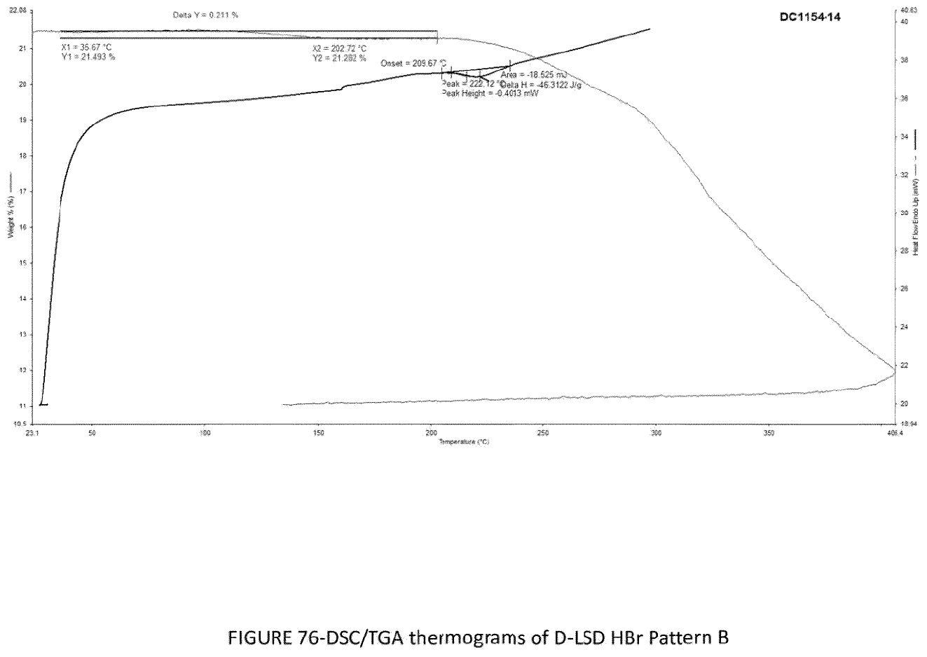

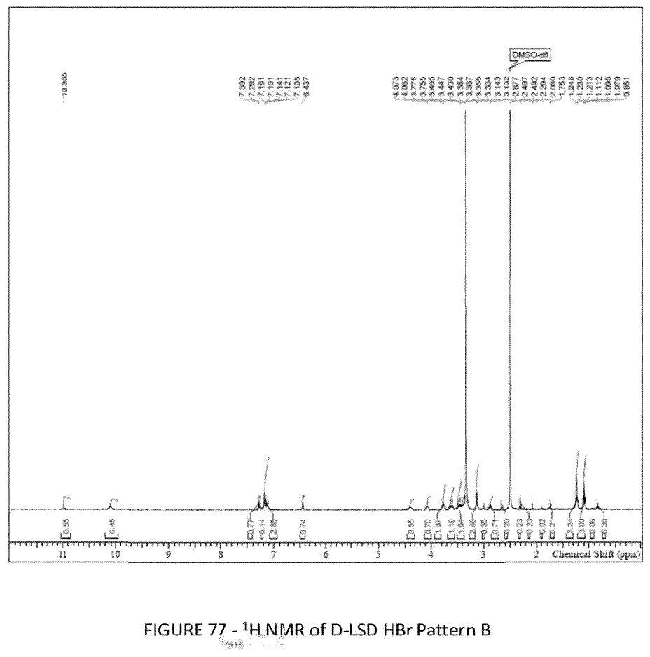

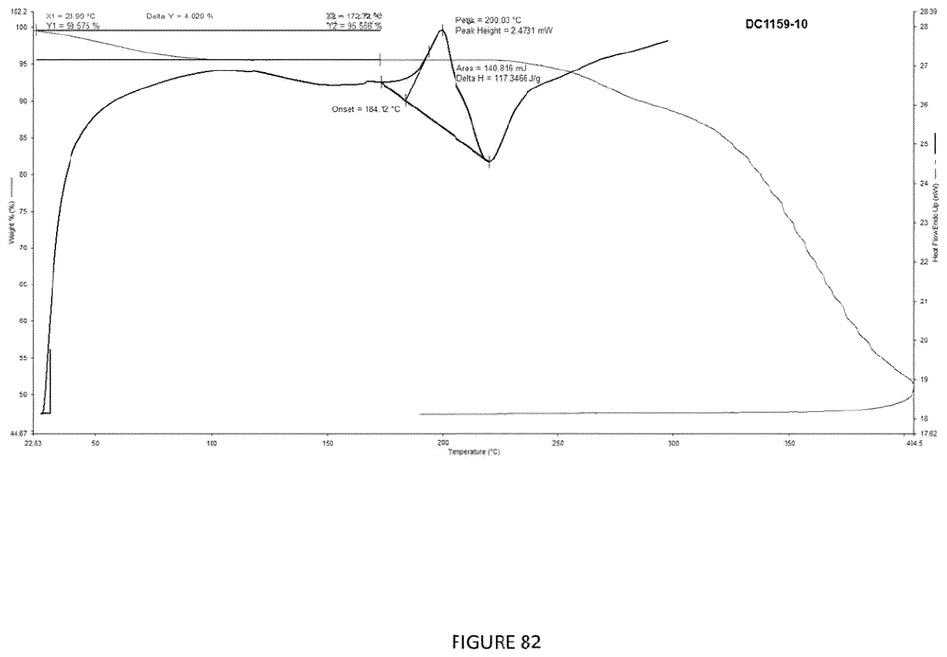

A sample of D-LSD HBr Pattern A salt (lot: DC1159-10) isolated by antisolvent cooling crystallisation from MeOH/MTBE was characterized by DSC and TGA (FIG. 82). DSC analysis showed a melting onset at 184° C. TGA thermogram illustrated a weight loss of 4% (w/w) between 23-172° C. indicating that the material was a monohydrate salt. D-LSD HBr Pattern B was characterized by XRPD (FIG. 75) and DSC/TGA (FIG. 76). DSC analysis illustrated an exothoermic peak at 210° C. and a small bump due to an instrumental defect is shown at ~160° C. The thermogram is unusual because no endo events were observed. TGA thermogram showed a minimal weight loss of 0.21% (w/w) between 35-202° C. The lack of significant energetics for this salt version may relate to degree of crystallinity or other content that is imparting such behavior. The ¹H NMR spectrum (FIG. 77) conformed to the molecular structure and traces of impurities/solvents were detected.

### Example 45

### Characterization of D-LSD HBr Amorphous Salt

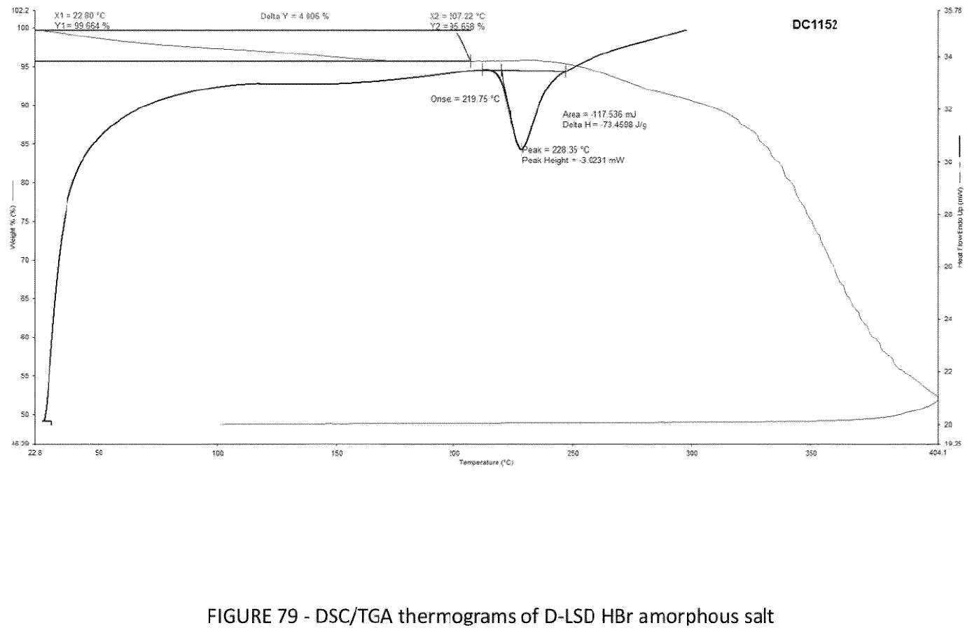

The amorphous D-LSD HBr salt (lot: DC1152) was characterized by DSC and TGA. DSC analysis showed an exotherm onset at 220° C. TGA analysis showed a weight loss of 4.0% (w/w) between 22-207° C. FIG. 79 shows DSC/TGA thermograms of D-LSD HBr salt.

### Example 46

### Solvent Vapour Diffusion of Amorphous D-LSD Sulfate (DC1155)

An investigation of the propensity of amorphous D-LSD sulfate to polymorphism was conducted using the solvent vapour diffusion technique with amorphous input. This technique uses a less forcing system and allows an API to order itself from surface interaction of solvent to induce crystallisation.

Experimental: Solvent vapour chambers were prepared by dosing solvent (3 ml, TABLE 14) into a glass vial. Into this vapour chamber was placed a smaller glass vial containing amorphous API (30 mg, DC1151). The chamber was sealed and allowed to equilibrate without disturbance for 2 weeks ahead of observation and XRPD analysis.

Results and observations are shown in TABLE 14 and most of the materials recovered were gels. D-LSD sulfate Pattern A was isolated once from cyclohexane (lot: DC1155-16). This material was characterized by XRPD, DSC, TGA and ¹H NMR.

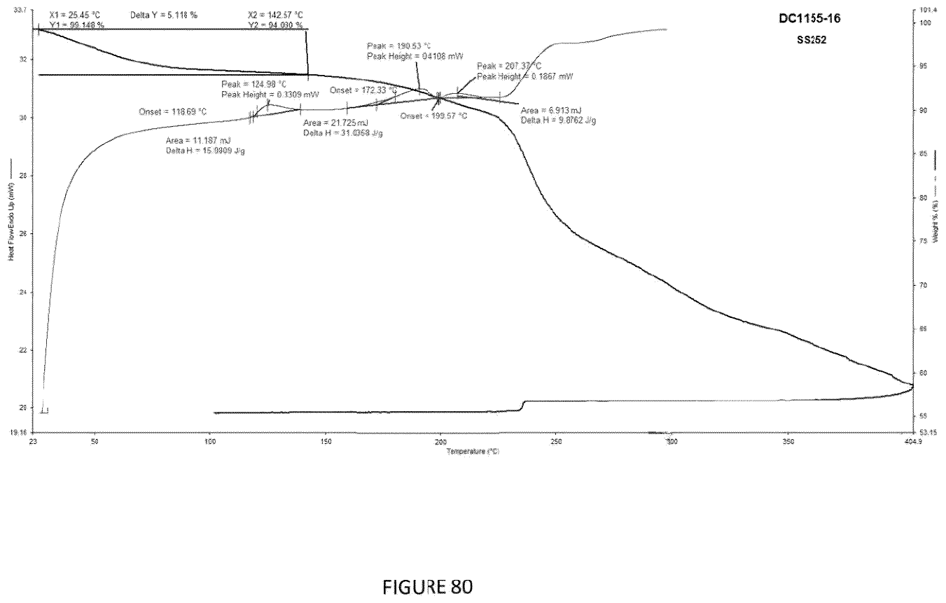

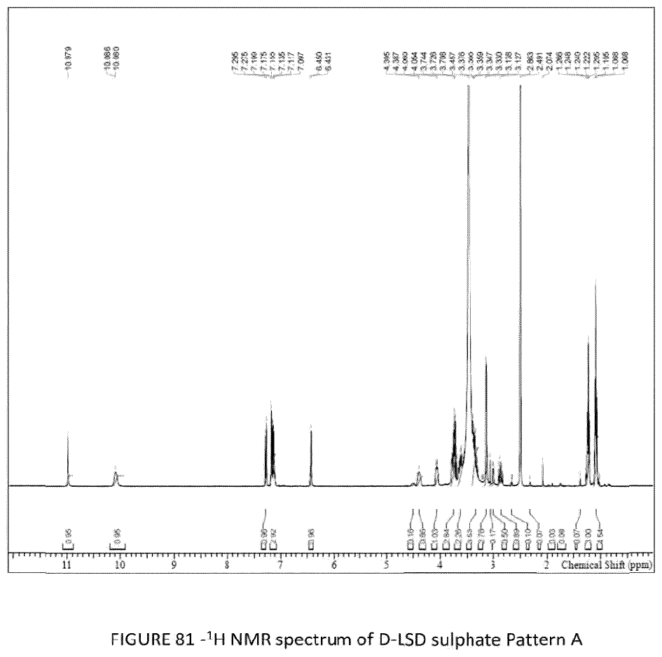

DSC analysis showed and endo onset at ~118° C. followed by two other endo onsets at 172 and 199° C. The TGA thermogram illustrated a weight loss of 5.1% (w/w) between 25-142° C. Stoichiometrically 1 mol eq. of water corresponded to 4.1% w/w, indicating a monohydrate salt. The ¹H NMR spectrum (FIG. 81) conformed to the molecular structure and traces of heptane were detected (0.006 mol eq.). FIG. 80 shows DSC/TGA thermograms of D-LSD sulfate Pattern A.

TABLE 14

| | | Observation | Observation | XRPD |
|---|---|---|---|---|
| Sample ID | Solvent | at 1 week | at 2 weeks | at 2 weeks |
| DC1155-1 | TBME | solid | solid | amorphous |
| DC1155-2 | THF | gel | gel | gel |
| DC1155-3 | 2-Me—THF | gel | gel | gel |
| DC1155-4 | 1,4-dioxane | gel | gel | gel |
| DC1155-5 | EtOAc | gel | gel | gel |
| DC1155-6 | IPAC | gel | gel | gel |
| DC1155-7 | acetone | gel | gel | gel |
| DC1155-8 | MEK | gel | gel | gel |
| DC1155-9 | MIBK | gel | gel | gel |
| DC1155-10 | anisole | gel | gel | gel |
| DC1155-11 | toluene | gel | gel | gel |
| DC1155-12 | chlorobenzene | gel | gel | gel |
| DC1155-13 | MeCN | gel | gel | gel |
| DC1155-14 | propionitrile | gel | gel | gel |
| DC1155-15 | heptane | gel | gel | gel |
| DC1155-16 | cyclohexane | solid | solid | sulfate |
| | | | | Pattern A |
| DC1155-17 | nitromethane | gel | gel | gel |
| DC1155-18 | DCM | gel | gel | gel |

### Anti-Solvent Mediated Crystallization of D-LSD Sulfate Salt (DC1158)

Experimental: D-LSD sulfate salt stock solutions were prepared by dissolving the material (200 mg, DC1151) in the minimum amount of MeOH and EtOH. The stock solutions were then clarified into clean sample vials. Aliquots (25 mg API) of the stock solutions were placed into 14 crystallisation tubes, pre-heated to 50° C. (30° C. for DCM). Anti-solvents, pre-heated to 50° C. were then added in 2 volume charges until a hazy solution was seen or until 20 volumes of anti-solvent were added. The solutions were then cooled and equilibrated at 25° C. for 24 hours.

Results and observations are presented in TABLE 15. Solutions and amorphous materials were recovered. The solutions were evaporated under ambient conditions and gels were mainly isolated.

TABLE 15

| | | | | Observations | | Observations |
|---|---|---|---|---|---|---|
| | | | Volume | at ambient | Form | after |
| ID | Solvent | Anti-solvent | added | temperature | by XRPD | evaporation |
| DC1158-1 | EtOH | heptane | 20 | solid | amorphous | N/A |
| DC1158-2 | (10 vol) | TBME | 20 | solution | — | glassy |
| DC1158-3 | | THF | 20 | solution | — | gel |
| DC1158-4 | | toluene | 5 | Solution | — | gel |
| DC1158-5 | | MEK | 20 | solution | — | glassy |
| DC1158-6 | | Ethyl acetate | 18 | solid | amorphous | N/A |
| DC1158-7 | | 1,4-dioxane | 20 | solution | — | gel |
| DC1158-8 | | acetone | 20 | solid | amorphous | N/A |
| DC1158-9 | MeOH | TBME | 20 | solution | — | gel |
| DC1158-10 | (10 vol) | THF | 20 | solution | — | gel |
| DC1158-11 | | toluene | 20 | solid | amorphous | N/A |
| DC1158-12 | | MEK | 20 | solid | amorphous | N/A |
| DC1158-13 | | Ethyl acetate | 20 | solution | — | gel |
| DC1158-14 | | 1,4-dioxane | 20 | solution | — | glassy |

### Polymorph Screen for D-LSD HBr Salt

### Solvent Vapour Diffusion of Amorphous D-LSD HBr Salt (DC1156)

An investigation of the propensity of amorphous D-LSD hydrobromide to polymorphism was conducted using the solvent vapour diffusion technique with amorphous input. This technique uses a less forcing system and allows an API to order itself from surface interaction of solvent to induce crystallization.

Experimental: Solvent vapour chambers were prepared by dosing solvent (3 ml, TABLE 16) into a glass vial. Into this vapour chamber was placed a smaller glass vial containing amorphous API (30 mg, DC1152). The chamber was sealed and allowed to equilibrate without disturbance for 2 weeks ahead of observation and XRPD analysis.

Results and observations are shown in TABLE 16. The amorphous materials were mainly recovered, and D-LSD HBr Pattern A was isolated twice from MeCN and cyclohexane.

TABLE 16

| | | Observation | Observation | XRPD |
|---|---|---|---|---|
| Sample ID | Solvent | at 1 week | at 1 weeks | at 2 weeks |
| DC1156-1 | TBME | solid | solid | amorphous |
| DC1156-2 | THF | gel | gel | — |
| DC1156-3 | water | gel | gel | — |
| DC1156-4 | 2-Me—THF | gel | gel | — |
| DC1156-5 | EtOAc | solid | solid | amorphous |
| DC1156-6 | IPAC | solid | solid | amorphous |
| DC1156-7 | acetone | solid | solid | amorphous + |
| | | | | peak |
| DC1156-8 | MIBK | gel | gel | — |
| DC1156-9 | anisole | solid | solid | amorphous |
| DC1156-10 | toluene | solid | solid | amorphous |
| DC1156-11 | chlorobenzene | solid | solid | amorphous |
| DC1156-12 | MeCN | solid | solid | Pattern A |
| DC1156-13 | heptane | solid | solid | amorphous |
| DC1156-14 | cyclohexane | solid | solid | Pattern A |

### Anti-Solvent Mediated Crystallization of D-LSD HBr Salt (DC1159)

Anti-solvent mediated crystallisation is a useful method for identifying new forms or versions of a solid as once dissolved, the API has no form memory and can recrystallize without bias. Solubility assessment of the API during the initial solvent maturation experiment had identified several solvents and anti-solvents to be used.

Experimental: D-LSD HBr salt stock solutions were prepared by dissolving the material (200 mg, DC1152) in the minimum amount of MeOH and EtOH. The stock solutions were then clarified into clean sample vials. Aliquots (25 mg API) of the stock solutions were placed into 16 crystallisation tubes, pre-heated to 50° C. (30° C. for DCM). Anti-solvents, pre-heated to 50° C. were then added in 2 volume charges until a hazy solution was seen or until 20 volumes of anti-solvent were added. The solutions were then cooled and equilibrated at 25° C. for 24 hours.

Results and observations are presented in TABLE 17. XRPD analysis showed that Pattern A was mainly isolated. The solution recovered after the anti-solvent cooling crystallisation experiments were slowly evaporated. Gels were mainly isolated.

TABLE 17

| | | | | Observations | | Observations |
|---|---|---|---|---|---|---|
| | | | Volume | at ambient | Form | after |
| ID | Solvent | Anti-solvent | added | temperature | by XRPD | evaporation |
| DC1159-1 | EtOH | heptane | 15 | solid | Pattern A | N/A |
| DC1159-2 | (10 vol) | TBME | 18 | solid | Pattern A | N/A |
| DC1159-3 | | THF | 20 | solution | — | gel |
| DC1159-4 | | toluene | 20 | solution | — | gel |
| DC1159-5 | | DCM | 20 | solution | — | gel |
| DC1159-6 | | Ethyl acetate | 20 | solid | Pattern A | N/A |
| DC1159-7 | | cyclohexane | 11 | solid | Pattern A | N/A |
| DC1159-8 | | acetone | 20 | solution | — | glassy |
| DC1159-9 | MeOH | heptane | 20 | solid | amorphous | N/A |
| DC1159-10 | (10 vol) | TBME | 20 | solid | Pattern A | N/A |
| DC1159-11 | | THF | 20 | solution | — | gel |
| DC1159-12 | | toluene | 20 | solid | amorphous | N/A |
| DC1159-13 | | DCM | 20 | solution | — | gel |
| DC1159-14 | | Ethyl acetate | 20 | solid | amorphous | N/A |
| DC1159-15 | | cyclohexane | 20 | solid | amorphous | N/A |
| DC1159-16 | | acetone | 20 | solution | — | glassy |

### Example 47

### Polymorph Screen for D-LSD Sulfate Salt

### Mixed Solvent Cooling Crystallizations of Amorphous D-LSD Sulfate Salt (DC1160)

Investigations conducted during the programme of works identified a range of solvents and anti-solvents of the API which can be exploited to provide a variety of modes of crystallization. The form fate of D-LSD sulfate salt (lot: DC1151) was further assessed by a series of mixed solvent cooling crystallizations.

Experimental: amorphous D-LSD sulfate (DC1151, 21×25 mg) was weighed into crystallisation tubes and suspended in the relevant anti-solvent (TABLE 18). Temperature was increased to 50° C. and solvent was added in aliquots until a solution was achieved. Solutions were cooled and equilibrated at room temperature for 16 hours. Solids were isolated by filtration and dried in vacuo at 40° C. for 18 hours prior to data collection. If solutions were observed, they were placed in a fridge at 0° C. for 24 hours and the recovered suspensions were filtered and analysed by XRPD. The solutions recovered after cooling were evaporated under ambient conditions and the solids observed were analysed by XRPD. Gel samples and amorphous materials were recovered.

TABLE 18

| | | | Volume | | Observation | |
|---|---|---|---|---|---|---|
| | | | solvent | Observation | after Slow | |
| Entry | Solvent | Anti-Solvent | added | at 0° C. | evaporation | XRPD |
| DC1160-1 | EtOH | TBME | 8 | solution | solid | amorphous |
| DC1160-2 | | THF | 5 | solution | gel | — |
| DC1160-3 | | 1,4-dioxane | 2 | solution | gel | — |
| DC1160-4 | | EtOAc | 3 | solution | gel | — |
| DC1160-5 | | IPAC | 3 | solution | solid | amorphous |
| DC1160-6 | | acetone | 2 | solution | solid | amorphous |
| DC1160-7 | | MEK | 2 | solution | solid | amorphous |
| DC1160-13 | | p-cymene | 5 | solution | gel | — |
| DC1160-14 | | heptane | 3 | solution | gel | — |
| DC1160-15 | | cyclohexane | 3 | solution | gel | — |
| DC1160-08 | MeOH | MIBK | 3 | solution | solid | amorphous |
| DC1160-11 | | chlorobenzene | 3 | solution | gel | — |
| DC1160-12 | | cumene | 2 | solution | solid | amorphous |
| DC1160-13 | | toluene | 2 | solution | gel | — |
| DC1160-14 | | heptane | 3 | solution | gel | — |
| DC1160-15 | | cyclohexane | 3 | solution | solid | amorphous |
| DC1160-16 | | pentane | 1 | solution | gel | — |
| DC1160-17 | | TBME | 3 | solution | gel | — |
| DC1160-18 | water | DCM | 6 | solution | gel | — |
| DC1160-19 | | THF | 1 | solution | gel | — |
| DC1160-20 | | 1,4-dioxane | 3 | solution | gel | — |

### Polymorph Screen for D-LSD HBr Salt

### Mixed Solvent Cooling Crystallizations of Amorphous D-LSD HBr Salt (DC1161)

Investigations conducted during the programme of works identified a range of solvents and anti-solvents of the API which can be exploited to provide a variety of modes of crystallization. The form fate of D-LSD HBr salt (lot: DC1152) was further assessed by a series of mixed solvent cooling crystallizations.

Experimental: amorphous D-LSD HBr (DC1152, 17×25 mg) was weighed into crystallisation tubes and suspended in the relevant anti-solvent (detailed in TABLE 19) (10 vol). Temperature was increased to 50° C. and solvent (detailed in TABLE 19) was added in aliquots until a solution was achieved. Solutions were cooled and equilibrated at room temperature for 16 hours. Solids were isolated by filtration and dried in vacuo at 40° C. for 18 hours prior to data collection. If solutions were observed, they were placed in a fridge at 0° C. for 24 hours and the recovered suspensions were filtered and analysed by XRPD. The solutions recovered after cooling were evaporated under ambient conditions and the solids observed were analysed by XRPD.

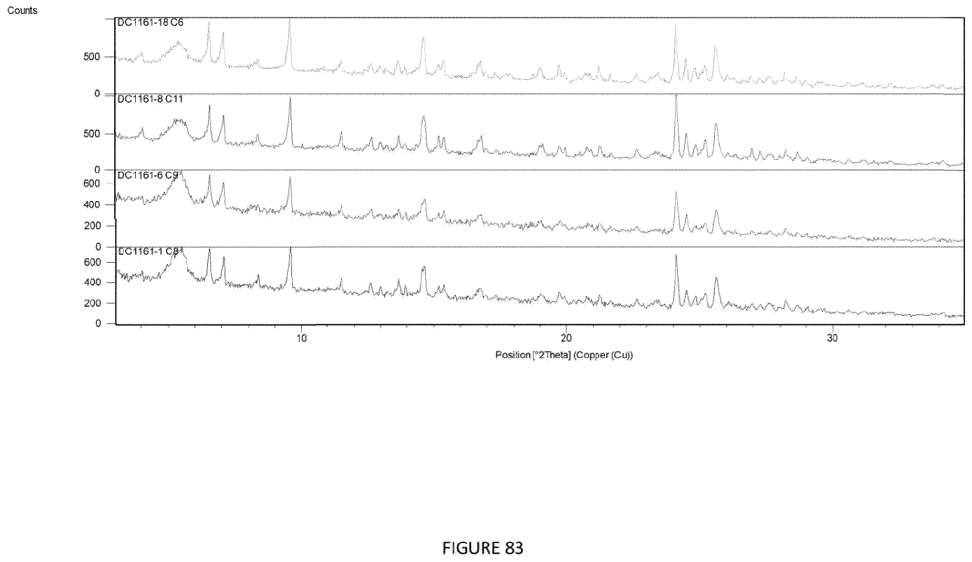

Results and observations shown in TABLE 19. Gels samples and amorphous materials were mainly recovered. D-LSD sulfate Pattern A was isolated from EtOH/TBME, EtOH/IPAC, EtOH/heptane and MeOH/MIBK. XRPD patterns of the isolated D-LSD sulfate Pattern A solids are shown in FIG. 83.

TABLE 19

| | | | Volume | | | | | |
|---|---|---|---|---|---|---|---|---|
| | | Anti- | solvent | Obs | Obs | Observation | Slow | |
| Entry | Solvent | Solvent | added | 50° C. | RT cool | at 0° C. | evap. | XRPD |
| DC1161-1 | EtOH | TBME | 4 | hazy | suspension | — | — | Pattern A |
| DC1161-2 | | THF | 2 | solution | solution | solution | gel | — |
| DC1161-3 | | water | 5 | solution | solution | solution | gel | — |
| DC1161-5 | | EtOAc | 6 | solution | solution | solution | gel | — |
| DC1161-6 | | IPAC | 5 | hazy | suspension | — | — | Pattern A |
| DC1161-7 | | acetone | 2 | solution | solution | solution | solid | amorphous |
| DC1161-18 | | heptane | 4 | solution | suspension | — | — | Pattern A |
| DC1161-8 | MeOH | MIBK | 2 | solution | suspension | — | — | Pattern A |
| DC1161-9 | | cumene | 2 | hazy | suspension | — | solid | amorphous |
| DC1161-10 | | toluene | 2 | solution | suspension | — | solid | amorphous |
| DC1161-11 | | chlorobenzene | 2 | hazy | suspension | — | solid | amorphous |
| DC1161-12 | | pentane | 2 | solution | solution | solution | gel | — |
| DC1161-13 | | heptane | 2 | hazy | suspension | — | — | amorphous |
| DC1161-15 | | p-cymene | 3 | solution | solution | solution | gel | — |
| DC1161-14 | water | cyclohexane | 5 | hazy | suspension | — | — | amorphous + |
| | | | | | | | | peaks |
| DC1161-16 | | THF | 1 | solution | solution | solution | gel | — |
| DC1161-20 | | DCM | 2 | solution | solution | solution | solid | amorphous |

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1-52. (canceled)

53. A crystalline salt form of LSD comprising D-LSD free-base and an acid, wherein the acid is maleic acid, phosphoric acid, benzoic acid, or xinafoic acid.

54. The crystalline salt form of claim 53, wherein said acid is maleic acid, and said crystalline salt form is characterized by an x-ray powder diffraction pattern (XRPD) obtained by irradiation with Cu Kα x-rays having peaks expressed as 2θ at about 17.4 and about 22.1.

55. The crystalline salt form of claim 54, wherein the crystalline salt form is further characterized by one or more XRPD signals expressed as 2θ at about 8.4, about 14.7, or about 15.1.

56. The crystalline salt form of claim 54, wherein the crystalline salt form is characterized by an XRPD pattern as shown in FIG. 4.

57. The crystalline salt form of claim 54, wherein the crystalline salt form is further characterized by a TGA pattern as shown in FIG. 34.

58. The crystalline salt form of claim 54, wherein the crystalline salt form is further characterized by a DSC profile as shown in FIG. 34.

59. The crystalline salt form of claim 53, wherein said acid is phosphoric acid, and said crystalline salt form C is characterized by an x-ray powder diffraction pattern (XRPD) obtained by irradiation with Cu Kα x-rays having peaks expressed as 2θ at about 15.0 and about 21.1.

60. The crystalline salt form of claim 59, wherein the crystalline salt form is further characterized by one or more XRPD signals expressed as 2θ at about 7.8, about 8.5, about 12.8, or about 15.1.

61. The crystalline salt form of claim 59, wherein the crystalline salt form is characterized by an XRPD pattern as shown in FIG. 37.

62. The crystalline salt form of claim 59, wherein the crystalline salt form is further characterized by a DSC thermogram as shown in FIG. 36.

63. The crystalline salt form of claim 53, wherein said acid is benzoic acid, and said crystalline salt form A is characterized by an x-ray powder diffraction pattern obtained by irradiation with Cu Kα x-rays having peaks expressed as 2θ at about 8.5 and about 16.6.

64. The crystalline salt form of claim 63, wherein the crystalline salt form is further characterized by one or more XRPD signals expressed as 2θ at about 14.6, about 18.5, or about 19.0.

65. The crystalline salt form of claim 63, wherein the crystalline salt form is characterized by an XRPD pattern as shown in FIG. 10.

66. The crystalline salt form of claim 63, wherein the crystalline salt form is further characterized by a TGA pattern as shown in FIG. 25.

67. The crystalline salt form of claim 63, wherein the crystalline salt form is further characterized by a DSC profile as shown in FIG. 25.

68. The crystalline salt form of claim 53, wherein said acid is xinafoic acid, and said crystalline salt form is characterized by an x-ray powder diffraction pattern obtained by irradiation with Cu Kα x-rays having peaks expressed as 2θ at about 15.6 and about 21.6.

69. The crystalline salt form of claim 68, wherein the crystalline salt form is further characterized by one or more XRPD signals expressed as 2θ at about 10.2, about 11.6, or about 13.3.

70. The crystalline salt form of claim 68, wherein the crystalline salt form is characterized by an XRPD pattern as shown in FIG. 5.

71. A pharmaceutical composition comprising the crystalline salt form of claim 53, and one or more pharmaceutically acceptable excipients.

72. A method of treating anxiety, comprising administering to a subject in need thereof the crystalline salt form of claim 53.

73. A method of treating depression, comprising administering to a subject in need thereof the crystalline salt form of claim 53.
